(19) United States
(12) Patent Application Publication
(10) Pub. No.: US 2023/0272364 A1
(43) Pub. Date: Aug. 31, 2023

(54) GLUCONATE DEHYDRATASE ENZYMES AND RECOMBINANT CELLS

(71) Applicant: BP Corporation North America Inc., Houston, TX (US)

(72) Inventor: Fernando Valle, San Diego, CA (US)

(73) Assignee: BP Corporation North America Inc., Houston, TX (US)

(21) Appl. No.: 17/613,898

(22) PCT Filed: Jul. 13, 2020
(86) PCT No.: PCT/US2020/041801

§ 371 Date: Jun. 17, 2022

Related U.S. Application Data
(60) Provisional application No. 62/872,817, filed on Jul. 11, 2019.

(51) Int. Cl. C12N 9/88; C12P 7/42; C12P 7/40; C12P 7/24

(52) U.S. Cl. CPC C12N 9/88; C12P 7/24; C12P 7/40; C12P 7/42; C12Y 402/01039

(57) ABSTRACT

Abstract: Gluconate dehydratase enzymes and recombinant cells are provided, along with their use in the production of 2-ke-to-3-deoxy-D-gluconate (KDG).

### CROSS REFERENCE TO RELATED APPLICATIONS

This patent application claims the benefit of priority of U.S. Provisional Pat. Application No. 62/872,817, filed Jul. 11, 2019, which is incorporated herein by reference in its entirety.

### SEQUENCE LISTING

This application is being filed electronically via EFS-Web and includes an electronically submitted Sequence Listing in .txt format. The .txt file contains a sequence listing entitled “BPC-007US-SL” created on Jun. 10, 2022 and is 63,385 bytes in size. The Sequence Listing contained in this .txt file is part of the specification and is hereby incorporated by reference herein in its entirety.

### FIELD OF THE INVENTION

The disclosure relates to gluconate dehydratase enzymes and recombinant cells for the production of 2-dehydro-3-deoxy-D-gluconate (KDG).

### BACKGROUND

2-dehydro-3-deoxy-D-gluconate (otherwise known as 2-keto-3-deoxy-D-gluconate; “KDG”) is of significant commercial interest because it can be used to prepare a variety of commercially important compounds. For example, KDG can be converted to 2′-deoxynucleosides which may be used as starting materials for the synthesis of antiviral and anticancer drugs. Methods of producing 2′deoxynucleosides from KDG are known in the art, see for example US 7,858,775. KDG can also be used as a substrate for the production of furan-derivatives such as 5-hydroxymethyl-2-furoic acid (HMFA), and furan dicarboxylic acid (FDCA) which may be used e.g. as starting materials for the production of renewable polymers. These furan-derivatives are traditionally produced from fructose which is more expensive and generally less available than other sugars, e.g. glucose. It has recently been shown that glucose can be used as a substrate for the production of KDG which can then be used as a substrate for the preparation of furan-derivatives including HMFA and FDCA. Methods to produce furan derivatives from glucose, involving KDG as an intermediate, are described in WO2016/141148. WO2017/030668 also demonstrates the use of KDG in the production of FDCA.

Cell-free metabolic reactions that metabolize glucose to pyruvate, and involve KDG as an intermediate product, have been proposed for the in vitro production of valuable chemicals, e.g. ethanol, isobutanol, lactate, malate etc. (Guterl J. et al., (2012) and Taniguchi et al., (2017)). This concept was demonstrated by using a set of partially purified enzymes from different microorganisms. Importantly, several of the enzymes in the tested pathways were from thermophilic microorganisms, and their optimal enzymatic activity was in the range of 63° C. to 99° C., while other enzymes used in the tested pathways were from mesophilic microorganisms, with optimal temperatures below 45° C. The combination of thermophilic enzymes and mesophilic enzymes in the same pathway resulted in a complex experimental design with several enzymes required to operate at suboptimal temperatures. For example, the two enzymes used to convert glucose into KDG were from Sulfolobus solfataricus and had an optimal temperature of 70° C., but the experimental design required their use at a suboptimal temperature of 50° C. (Guterl J. et al. (2012)).

It is not commercially feasible to carry out enzymatic production of KDG at thermophilic temperatures, because such high temperatures lead to KDG decomposition and the formation of unwanted by-products.

A one-step in vitro synthesis of KDG from gluconate has been reported using a purified gluconate dehydratase from the hyperthermophilic archaea Thermoproteus tenax, which has an optimal growth temperature of 86° C. (Matsubara Kl. et al. (2014)). In an attempt to overcome the significant problems associated with enzymatic production of KDG at such high temperatures, the authors were required to perform the enzymatic reaction at the suboptimal temperature of 50° C. As a consequence of performing the reaction at a suboptimal temperature, a laboratory scale 1.5 L reaction containing 7 µg/ml of purified gluconate dehydratase required 117 hours to achieve full conversion of 3.3 g of gluconate to KDG. The authors acknowledge that higher conversion rates would be achieved using higher reaction temperatures, but a reaction temperature of 50° C. had to be used to prevent the formation of unwanted by-products. In addition to prolonged reaction times (as compared to reactions performed at an optimal enzyme temperature), this approach had two additional major drawbacks: (1) using the gluconate dehydratase at a suboptimal temperature required high enzyme concentrations to achieve useful KDG production; and (2) the process required the use of purified gluconate as a substrate, which is expensive.

Kim & Lee (2008) identified and characterized a gluconate dehydratase from Achromobacter xylosoxidans ATCC9220 which was expressed in Escherichia coli. This enzyme was reported to display broad substrate specificity, with a preference for gluconate and an optimal temperature of 50° C. However, its activity decreased to 50% after 60 min at 50° C. At 37° C. (i.e. the optimal growth temperature for E. coli) the enzyme had approximately ~50% of its optimal activity. The same A. xylosoxidans gluconate dehydratase is also mentioned in US 7,125,704 and US 7,510,861 which indicate a preferred experimental temperature range of 50-60° C. US 7,125,704 and US 7,510,861 also use E. coli as a host organism for gluconate dehydratase expression. As the optimal growth temperature for E. coli is 37° C., and the optimal temperature of the heterologously expressed gluconate dehydratase is 50° C., the authors of US 7,125,704 and US 7,510,861 were forced to conduct two separate incubation steps to improve the yield of KDG. First, the E. coli cells were grown at 37° C. Second, the cells were then collected by centrifugation, added to a reaction mixture containing gluconate, and incubated at 50° C. to produce KDG. A major disadvantage to this approach is that useful production of KDG requires that cell growth be interrupted (e.g. arrested or suppressed) which significantly limits the overall efficiency of the process.

There is an urgent and unmet need for efficient production of KDG which does not sacrifice optimal enzyme activity in order to avoid decomposition of KDG and/or the formation of unwanted by-products. Likewise, there is an urgent and unmet need for efficient production of KDG which does not require interruption (e.g. arrestation or suppression) of cell division and growth.

### SUMMARY OF THE INVENTION

The present disclosure provides gluconate dehydratase enzymes and recombinant cells for the production of KDG. The enzymes and recombinant cells of the disclosure possess highly desirable characteristics including e.g.: (i) efficient KDG production at temperatures that retain stability of KDG and substantially avoid the production of unwanted by-products; and (ii) production of commercially desirable yields of KDG without requiring interruption of cell growth. Moreover, the gluconate dehydratase enzymes and recombinant cells of the disclosure are ideally suited to the use of glucose as a substrate, which is readily available and at low cost.

The disclosure provides a recombinant cell comprising a gene encoding a heterologous gluconate dehydratase, wherein the recombinant cell further comprises one or more genetic modifications resulting in at least one, any two, any three, any four, or all five of the following phenotypes: (a) decrease or elimination of glucose assimilation; (b) decrease or elimination of gluconate-6-phosphate production from gluconate; (c) decrease or elimination of glucose-6-phosphate production from glucose; (d) decrease or elimination of 2-keto-3-deoxy-D-gluconate (KDG) phosphorylation; and (e) decrease or elimination of KDG degradation by non-phosphorylative cellular reactions that consume KDG.

The disclosure also provides a recombinant cell comprising a genetic modification resulting in the decrease or elimination of KDG phosphorylation, wherein the recombinant cell further comprises one or more genetic modifications resulting in at least one, any two, any three, or all four of the following phenotypes: (a) decrease or elimination of glucose assimilation; (b) decrease or elimination of gluconate-6-phosphate production from gluconate; (c) decrease or elimination of glucose-6-phosphate production from glucose; and (d) decrease or elimination of KDG degradation by non-phosphorylative cellular reactions that consume KDG.

In certain embodiments, said one or more genetic modifications resulting in decrease or elimination of: (a) glucose assimilation comprises deletion or inactivation of at least one gene involved in the PEP-dependent carbohydrate-phosphotransferase system; (b) gluconate-6-phosphate production from gluconate comprises deletion or inactivation of at least one gene encoding a gluconate kinase; (c) glucose-6-phosphate production from glucose comprises deletion or inactivation of at least one gene encoding a glucokinase; (d) KDG phosphorylation comprises deletion or inactivation of 2-dehydro-3-deoxygluconokinase; and/or (e) KDG degradation by non-phosphorylative cellular reactions that consume KDG comprises deletion or inactivation of a KDG aldolase.

In certain embodiments, said one or more genetic modifications resulting in decrease or elimination of: (a) glucose assimilation comprises deletion or inactivation of ptsl, ptsH and/or crr; (b) gluconate-6-phosphate production comprises deletion or inactivation of gntK; (c) gluconate-6-phosphate production comprises deletion or inactivation of idnK; (d) glucose-6-phosphate production comprises deletion or inactivation of glk; (e) KDG phosphorylation comprises deletion or inactivation of kdgK; and/or (f) KDG degradation by non-phosphorylative cellular reactions that consume KDG comprises deletion or inactivation of yjhH and/or yagE.

In certain embodiments, the recombinant cell of the disclosure further comprises one or more genetic modifications resulting in decreased uptake of KDG into the recombinant cell. In certain embodiments, said one or more genetic modifications resulting in decreased uptake of KDG into the recombinant cell comprises deletion or inactivation of 2-keto-3-deoxygluconate permease. In certain embodiments, said one or more genetic modifications resulting in decreased uptake of KDG into the recombinant cell comprises deletion or inactivation of kdgT.

In certain embodiments, the recombinant cell of the disclosure further comprises one or more genetic modifications resulting in increased uptake of gluconate into the recombinant cell. In certain embodiments, said one or more genetic modifications resulting in increased uptake of gluconate into the recombinant cell comprises increasing expression of a gluconate transporter. In certain embodiments, said one or more genetic modifications resulting in increased uptake of gluconate into the recombinant cell comprises increasing expression of gntP, gntU, gntT, and/or idnT. In certain embodiments, said one or more genetic modifications resulting in increased uptake of gluconate into the recombinant cell comprises increasing expression of gntT and/or gntP.

In certain embodiments, the recombinant cell comprises a gene encoding a heterologous gluconate dehydratase, wherein the gene encodes a polypeptide comprising an amino acid sequence having at least 70% sequence identity to SEQ ID NO: 1.

In certain embodiments, the recombinant cell comprises a gene encoding a heterologous gluconate dehydratase, wherein the gene comprises a nucleic acid sequence having at least 70% sequence identity to SEQ ID NO: 2 or 3. In certain embodiments, the recombinant cell comprises a gene encoding a heterologous gluconate dehydratase, wherein the gene comprises a nucleic acid sequence having at least 70% sequence identity to SEQ ID NO: 4.

In certain embodiments, the recombinant cell comprises a vector, wherein said vector comprises a gene encoding a heterologous gluconate dehydratase polypeptide comprising an amino acid sequence having at least 70% sequence identity to SEQ ID NO: 1.

In certain embodiments, the recombinant cell comprises a vector, wherein said vector comprises a gene encoding a heterologous gluconate dehydratase comprising a nucleic acid sequence having at least 70% sequence identity to SEQ ID NO: 2, 3 or 4.

In certain embodiments, the recombinant cell comprises a vector, wherein the vector comprises: (a) an origin of replication; (b) a promoter sequence operably linked to said gene; and/or (c) a reporter gene.

In certain embodiments, the recombinant cell comprises a vector, wherein the vector comprises a sequence having at least 70% sequence identity to SEQ ID NO: 5. In certain embodiments, the recombinant cell comprises a vector, wherein the vector comprises a sequence having at least 70% sequence identity to SEQ ID NO: 6. In certain embodiments, the recombinant cell comprises a vector, wherein the vector comprises a sequence having at least 70% sequence identity to SEQ ID NO: 7. In certain embodiments, the recombinant cell comprises a vector, wherein the vector comprises a sequence having at least 70% sequence identity to SEQ ID NO: 8.

In certain embodiments, the recombinant cell is stably transformed with the gene encoding a heterologous gluconate dehydratase. In certain embodiments, the recombinant cell is transiently transformed with the gene encoding a heterologous gluconate dehydratase.

In certain embodiments, the recombinant cell is a prokaryotic cell. In certain embodiments, the prokaryotic cell is selected from the group consisting of Acinetobacter, Agrobacterium, Escherichia, Cupriavidus, Clostridium, Rhodobacter, Marinobacter, Bacillus, Klebsiella, Tatumella, Pseudomonas, Ralstonia, Rhodococcus, Methylobacterium, Methylophilus, Methylococcus, Methylomicrobium, Methylomonas, Pantoea, Streptomyces, Zymomonas, Parachlorella, Synechococcus, Synechocystis, and Thermocynechococcus. In certain embodiments, the prokaryotic cell is E. coli.

In certain embodiments, the recombinant cell is a eukaryotic cell. In certain embodiments, the eukaryotic cell is selected from the group consisting of a yeast cell, a fungal cell, and an algal cell. In certain embodiments, he yeast cell is selected from the group consisting of Saccharomyces, Kluyveromyces, Candida, Pichia, Schizosaccharomyces, Hansenula, Klockera, Schwanniomyces, Schefferomyces, Rhodosporidium, Issatchenkia, Yarrowia and Rhodotorula. In certain embodiments, the yeast cell is selected from the group consisting of S. cerevisiae, C. lipolytica, R. glutinis, S. bulderi, S. barnetti, S. exiguus, S. uvarum, S. diastaticus, K. lactis, K. marxianus, K. fragile, P. kudriavzevii, S. stipites, and I. orientalis.

In certain embodiments, the fungal cell is a filamentous fungal cell. In certain embodiments, the filamentous fungal cell is selected from the group consisting of Aspergillus, Penicillium, Rhizopus, Chrysosporium, Myceliophthora, Trichoderma, Humicola, Acremonium and Fusarium. In certain embodiments, the filamentous fungal cell is selected from the group consisting of A. niger, A. oryzae, T. reesei, P. chrysogenum, M. thermophila, and R. oryzae.

In certain embodiments, the algal cell is selected from the group consisting of Botryococcus, Nannochloropsis, Chlorella, Chlamydomonas, Dunaliella, Chaetoceros, Porphyridium, Scenedesmus and Pseudochlorococcum. In certain embodiments, the algal cell is selected from the group consisting of B. braunii and N. gaditana.

In certain embodiments, the recombinant cell of the disclosure further comprises genetic modification(s) resulting in one or more of the following phenotypes: (a) increased production of pyruvate and/or glyceraldehyde-3-phosphate from KDG; (b) increased production of isopentenyl pyrophosphate (IPP) and/or dimethylallyl pyrophosphate (DMAPP) from KDG; and (c) increased production of terpenoids from KDG.

In certain embodiments, the genetic modifications resulting in increased production of: (a) pyruvate and/or glyceralehyde-3-phosphate from KDG comprise increasing expression of a KDG/KDPG aldolase; (b) IPP or DMAPP from KDG comprise increasing expression of enzymes involved in the non-mevalonate (MEP) pathway; and/or (c) terpenoids from KDG comprise increasing expression of enzymes involved in the terpenoid biosynthetic pathway.

The disclosure also provides a method of producing 2-keto-3-deoxy gluconate (KDG) comprising the steps of: (a) culturing a recombinant cell in a suitable culture medium, wherein the recombinant cell comprises a gene encoding a heterologous gluconate dehydratase enzyme having at least 70% sequence identity to SEQ ID NO: 1; and (b) allowing expression of said gene, wherein said expression results in the production of KDG. Typically, production of KDG is performed at a temperature between 20° C. and 45° C.

In certain embodiments, the gene encoding a heterologous gluconate dehydratase comprises a nucleic acid sequence having at least 70% sequence identity to SEQ ID NO: 2 or 3. In certain embodiments, the gene encoding a heterologous gluconate dehydratase comprises a nucleic acid sequence having at least 70% sequence identity to SEQ ID NO: 4.

In certain embodiments, the culture medium comprises glycerol and glucose. In certain embodiments, the yield of KDG is at least 60%.

In certain embodiments, the yield of KDG from glucose is at least 60% when: (a) the recombinant cells are in stationary phase; (b) the culture medium comprises 10 g/L glucose; and (c) the culture is incubated for 46 hours at a temperature between 20° C. and 45° C., preferably at 37° C.

In certain embodiments, the yield of KDG from glucose is at least 60% when: (a) the recombinant cells are in stationary phase; and wherein the recombinant cells are E. coli comprising genetic modifications resulting in decrease or elimination of: (i) glucose assimilation, wherein said genetic modifications comprise deletion or inactivation of ptsl, ptsH and/or crr; (ii) gluconate-6-phosphate production, wherein said genetic modifications comprise deletion or inactivation of gntK; (iii) gluconate-6-phosphate production, wherein said genetic modifications comprise deletion or inactivation of idnK; (iv) glucose-6-phosphate production, wherein said genetic modifications comprise deletion or inactivation of glk; and (v) KDGphosphorylation, wherein said genetic modifications comprise deletion or inactivation of kdgK; (b) the culture medium comprises 10 g/L glucose; and (c) the culture is incubated for 46 hours at a temperature between 20° C. and 45° C., preferably at 37° C.

In certain embodiments, the yield of KDG from glucose is at least 60% when: (a) the recombinant cells are in log phase; (b) the culture medium comprises 10 g/L glucose; and (c) the culture is incubated for 48 hours at a temperature between 20° C. and 45° C., preferably at 37° C.

In certain embodiments, the yield of KDG from glucose is at least 60% when: (a) the recombinant cells are in log phase; and wherein the recombinant cells are E. coli comprising genetic modifications resulting in decrease or elimination of: (i) glucose assimilation, wherein said genetic modifications comprise deletion or inactivation of ptsl, ptsH and/or crr; (ii) gluconate-6-phosphate production, wherein said genetic modifications comprise deletion or inactivation of gntK; (iii) gluconate-6-phosphate production, wherein said genetic modifications comprise deletion or inactivation of idnK; (iv) glucose-6-phosphate production, wherein said genetic modifications comprise deletion or inactivation of glk; and (v) KDGphosphorylation, wherein said genetic modifications comprise deletion or inactivation of kdgK; (b) the culture medium comprises 10 g/L glucose; and (c) the culture is incubated for 48 hours at a temperature between 20° C. and 45° C., preferably at 37° C.

In certain embodiments, the method further comprises purifying KDG from cell culture. In certain embodiments, the method further comprises purifying KDG from supernatant. In certain embodiments, the method further comprises harvesting and lysing recombinant cell(s) to obtain KDG. In certain embodiments, the method further comprises purifying KDG from lysate.

In certain embodiments, the method further comprises converting KDG to a 2′-deoxynucleoside or a precursor thereof.

In certain embodiments, the method further comprises converting KDG to 5-hydroxymethyl-2-furoic acid (HMFA) and/or furan dicarboxylic acid (FDCA).

In certain embodiments, the recombinant cell is defined according to any one of claims 1 to 31.

In certain embodiments, the recombinant cell further comprises genetic modifications resulting in one or more of the following phenotypes: (a) increased production of pyruvate and/or glyceraldehyde-3-phosphate from KDG; (b) increased production of isopentenyl pyrophosphate (IPP) and/or dimethylallyl pyrophosphate (DMAPP) from KDG; and (c) increased production of terpenoids from KDG.

In certain embodiments, the genetic modifications resulting in increased production of: (a) pyruvate and/or glyceralehyde-3-phosphate from KDG comprises increasing expression of a KDG/KDPG aldolase; (b) IPP or DMAPP from KDG comprises increasing expression of enzymes involved in the non-mevalonate (MEP) pathway; and (c) terpenoids from KDG comprises increasing expression of enzymes involved in the terpenoid biosynthetic pathway.

In certain embodiments, the method further comprises a step of purifying from cell culture: (a) pyruvate and/or glyceraldehyde-3-phosphate; (b) IPP or DMAPP; and/or (c) terpenoids.

In certain embodiments, the method further comprises a step of purifying from supernatant: (a) pyruvate and/or glyceraldehyde-3-phosphate; (b) IPP or DMAPP; and/or (c) terpenoids.

In certain embodiments, the method further comprises harvesting and lysing recombinant cell(s) to obtain: (a) pyruvate and/or glyceraldehyde-3-phosphate; (b) IPP or DMAPP; and/or (c) terpenoids.

In certain embodiments, the method further comprises a step of purifying from lysate: (a) pyruvate and/or glyceraldehyde-3-phosphate; (b) IPP or DMAPP; and/or (c) terpenoids.

In certain embodiments, the method further comprises harvesting and extracting the cells with a solvent to obtain: (a) pyruvate and/or glyceraldehyde-3-phosphate; (b) IPP or DMAPP; and/or (c) terpenoids. In certain embodiments the solvent is an organic solvent, and in certain embodiments the solvent is immiscible in water, and in some embodiments, the solvent is an organic solvent that is immiscible in water.

The disclosure also provides a recombinant polypeptide comprising an amino acid sequence having at least 99% sequence identity to SEQ ID NO: 1.

The disclosure also provides a nucleic acid which encodes a polypeptide comprising an amino acid sequence having at least 99% sequence identity to SEQ ID NO: 1. Nucleic acids of the disclosure are typically isolated, recombinant or synthetic.

In certain embodiments, the nucleic acid is codon optimized for expression in a prokaryotic cell. In certain embodiments, the prokaryotic cell is selected from the group consisting of Acinetobacter, Agrobacterium, Escherichia, Cupriavidus, Clostridium, Rhodobacter, Marinobacter, Bacillus, Klebsiella, Tatumella, Pseudomonas, Ralstonia, Rhodococcus, Methylobacterium, Methylophilus, Methylococcus, Methylomicrobium, Methylomonas, Pantoea, Streptomyces, Zymomonas, Parachlorella, Synechococcus, Synechocystis and Thermocynechococcus. In certain embodiments, the prokaryotic cell is E. coli. In certain embodiments, the nucleic acid comprises a sequence having at least 70% sequence identity to SEQ ID NO: 4.

In certain embodiments, the nucleic acid sequence is codon optimized for expression in a eukaryotic cell. In certain embodiments, the eukaryotic cell is selected from the group consisting of a yeast cell, a fungal cell, and an algal cell.

In certain embodiments, the yeast cell is selected from the group consisting of Saccharomyces, Kluyveromyces, Candida, Pichia, Schizosaccharomyces, Hansenula, Klockera, Schwanniomyces, Schefferomyces, Rhodosporidium, Issatchenkia, Yarrowia and Rhodotorula. In certain embodiments, the yeast cell is selected from the group consisting of S. cerevisiae, C. lipolytica, R. glutinis, S. bulderi, S. barnetti, S. exiguus, S. uvarum, S. diastaticus, K. lactis, K. marxianus K. fragile, P. kudriavzevii, S. stipitis, and I. orientalis.

In certain embodiments, the fungal cell is a filamentous fungal cell. In certain embodiments, the filamentous fungal cell is selected from the group consisting of Aspergillus, Penicillium, Rhizopus, Chrysosporium, Myceliophthora, Trichoderma, Humicola, Acremonium and Fusarium. In certain embodiments, the filamentous fungal cell is selected from the group consisting of A. niger, A. oryzae, T. reesei, P. chrysogenum, M. thermophila, and R. oryzae.

In certain embodiments, the algal cell is selected from the group comprising Botryococcus, Nannochloropsis, Chlorella, Chlamydomonas, Dunaliella, Chaetoceros, Porphyridium, Scenedesmus and Pseudochlorococcum. In certain embodiments, the algal cell is selected from the group comprising B. braunii and N. gaditana.

In certain embodiments, the nucleic acid comprises a sequence having at least 70% sequence identity to SEQ ID NO: 2 or 3.

The disclosure also provides the use of a polypeptide comprising an amino acid sequence having at least 99% sequence identity to SEQ ID NO: 1 in a method of producing KDG.

The disclosure also provides a nucleic acid comprising a nucleic acid sequence having at least 70% sequence identity to SEQ ID NO: 4. Nucleic acids of the disclosure are typically isolated, recombinant or synthetic.

The disclosure also provides a vector comprising a nucleic acid sequence having at least 70% sequence identity to SEQ ID NO: 4. In certain embodiments, the vector comprises: (a) an origin of replication; (b) a promoter sequence operably linked to said nucleic acid comprising a nucleic acid sequence having at least 70% sequence identity to SEQ ID NO: 4; and/or (c) a reporter gene. In certain embodiments, the vector comprises a nucleic acid sequence having at least 70% sequence identity to SEQ ID NO: 5. In certain embodiments, the vector comprises a nucleic acid sequence having at least 70% sequence identity to SEQ ID NO: 7. In certain embodiments, the vector comprises a nucleic acid sequence having at least 70% sequence identity to SEQ ID NO: 6. In certain embodiments, the vector comprises a nucleic acid sequence having at least 70% sequence identity to SEQ ID NO: 8.

The disclosure also provides nucleic acid comprising a nucleic acid sequence having at least 70% sequence identity to SEQ ID NO: 2 or 3. Nucleic acids of the disclosure are typically isolated, recombinant or synthetic.

The disclosure also provides a vector comprising a nucleic acid sequence having at least 70% sequence identity to SEQ ID NO: 2 or 3. In certain embodiments, the vector comprises: (a) an origin of replication; (b) a promoter sequence operably linked to said nucleic acid comprising a nucleic acid sequence having at least 70% sequence identity to SEQ ID NO: 2 or 3; and/or (c) a reporter gene. In certain embodiments, the vector comprises a nucleic acid sequence having at least 70% sequence identity to SEQ ID NO: 5. In certain embodiments, the vector comprises a nucleic acid sequence having at least 70% sequence identity to SEQ ID NO: 7.

The disclosure also provides a recombinant cell comprising a vector, the vector comprising a nucleic acid sequence having at least 70% sequence identity to SEQ ID NO: 4. In certain embodiments, the vector comprises: (a) an origin of replication; (b) a promoter sequence operably linked to said nucleic acid comprising a nucleic acid sequence having at least 70% sequence identity to SEQ ID NO: 4; and/or (c) a reporter gene. In certain embodiments, the vector comprises a nucleic acid sequence having at least 70% sequence identity to SEQ ID NO: 5. In certain embodiments, the vector comprises a nucleic acid sequence having at least 70% sequence identity to SEQ ID NO: 7. In certain embodiments, the vector comprises a nucleic acid sequence having at least 70% sequence identity to SEQ ID NO: 6. In certain embodiments, the vector comprises a nucleic acid sequence having at least 70% sequence identity to SEQ ID NO: 8.

### BRIEF DESCRIPTION OF THE DRAWINGS

The foregoing aspects and many of the attendant advantages will become more readily appreciated as the same become better understood by reference to the following detailed description, when taken in conjunction with the accompanying drawings.

FIG. 1: Gene deletion method as described in Datsenko and Wanner (2000), and Baba et al., (2006).

FIG. 2: Gene arrangement and primer position of: (A) pstH, ptsl and crr; and (B) gntK.

FIG. 3: Gene arrangement and primer position of: (A) idnK; and (B) glk.

FIG. 4: Gene arrangement and primer position of kdgK.

FIG. 5: Schematic representation of the strategy used to identify the efficient gluconate dehydratase enzyme of the disclosure. In the first step, cells are grown on D-glucuronate to induce expression of enzymes involved in D-glucuronate metabolism. In the second step, cells are transferred to media containing gluconate as the sole carbon source. Only strains containing an active gluconate dehydratase enzyme will be able to produce KDG. Once formed, KDG is phosphorylated and metabolized via the central metabolic routes.

FIG. 6: Effect of different sugar dehydratases on the growth (OD₆₀₀) of Δ3 E. coli strains on gluconate. Strains 1-4 comprise a plasmid encoding the gluconate dehydratase of the disclosure; stain 5 comprises an empty plasmid i.e. without a sugar dehydratase; strains 6 and 7 comprise a plasmid expressing a D-glucarate dehydratase (GudD); strains 8 and 9 comprise a plasmid expressing a D-galactonate dehydratase (RspA); strains 10 and 11 comprise a plasmid expressing a mannonate dehydratase (UxuA); and strains 12 and 13 comprise a plasmid expressing a D-glucarate dehydratase (GudX).

FIG. 7: Effect of high and low copy number plasmids expressing gluconate dehydratase on the growth (OD₆₀₀) of Δ3 E. coli strains on gluconate. Strains 1, 2, 5 and 6 comprise the high copy number plasmid pAchro-10 encoding gluconate dehydratase of the disclosure. Strains 3, 4, 7 and 8 comprise the low copy number plasmid pAchro-L1 encoding gluconate dehydratase of the disclosure. Strains 1-4 were grown in the presence of gluconate, whereas strains 5-8 were grown in the absence of a carbon source.

FIG. 8: KDG metabolic routes are shown, a phosphorylated route, which leads to the formation of Glyceraldehyde-2-Phosphate and Pyruvate; and a non-phosphorylated route, which produces two molecules of Pyruvate.

### DETAILED DESCRIPTION OF THE INVENTION

### Gluconate Dehydratase Polypeptides

The present disclosure is based on the surprising discovery of a gluconate dehydratase that catalyses production of commercially desirable yields of KDG at mesophilic temperatures (i.e. at a temperature of 20-45° C.). The gluconate dehydratase identified by the inventors overcomes a long-felt need in the art for a gluconate dehydratase that can provide commercially desirable yields of KDG without requiring high reaction temperatures (e.g. temperatures greater than 50° C.). High reaction temperatures are problematic in the synthesis of KDG, not least because: (i) they lead to thermal degradation of KDG; and (ii) they lead to the formation of undesirable by-products. When KDG is synthesized in vivo, high temperatures are also problematic because they lead to reduced growth rates (or even death) of the host cell, thereby requiring thermocycling throughout the KDG production process, and/or additional lead-time to bulk-up cell numbers before further rounds of KDG biosynthesis may take place.

Gluconate dehydratases in the art have been identified in thermophilic microorganisms. Thermophilic microorganisms typically achieve optimal growth rates at temperatures in the range 45-120° C. Hyperthermophilic microorganisms are a subset of thermophilic microorganisms, and typically achieve optimal growth rates at temperatures in the range 70-120° C. Accordingly, gluconate dehydratases obtained from thermophilic (or hyperthermophillic) microorganisms typically exhibit optimal activity at thermophilic (or hyperthermophillic) temperatures (in the range of 45-120° C. or 70-120° C., respectively). This is why KDG production methods in the art typically perform catalytic reactions at high temperatures (e.g. 50° C. or higher). Lower catalytic reaction temperatures are typically avoided in the art because rates of catalysis are well-known to decrease with reduction in temperature.

The gluconate dehydratase of the disclosure was identified in silico in two uncharacterized bacteria, Achromobacter sp. 2789STDY5608625 and Achromobacter sp. K91, and was annotated as a dihydroxy-acid dehydratase (EC number 4.2.1.9). Dihydroxy-acid dehydratase catalyzes the dehydration of 2,3-dihydroxy-3-methylbutanoate into 3-methyl-2-oxobutanoate and H₂O, and participates in valine, leucine and isoleucine biosynthesis and pantothenate and coenzyme A (CoA) biosynthesis. Despite having high sequence identity to the thermophilic gluconate dehydratase from Achromobacter xylosoxidans ATCC9220 (see Kim and Lee 2008), the gluconate dehydratase of the disclosure can advantageously and unexpectedly provide commercially desirable yields of KDG at temperatures that avoid thermal degradation of KDG and can avoid formation of undesirable by-products (as can be suffered by methods in the prior art). Moreover, commercially desirable yields of KDG may be achieved using gluconate dehydratase of the disclosure at temperatures that are compatible with optimal growth of commercially relevant host microorganisms, such as E. coli. Advantageously, this also avoids the requirement to cycle between temperatures required for cell growth, and temperatures required for desirable levels of enzyme activity.

A gluconate dehydratase polypeptide of the disclosure comprises an amino acid sequence having at least 99% sequence identity to SEQ ID NO: 1. In some embodiments, the gluconate dehydratase polypeptide of the disclosure comprises an amino acid sequence having 100% sequence identity to SEQ ID NO: 1. In some embodiments, the gluconate dehydratase polypeptide of the disclosure comprises an amino acid sequence having at least 99% sequence identity to a functional fragment or catalytic domain of SEQ ID NO: 1. The gluconate dehydratase polypeptide may be isolated, synthetic, purified or recombinant.

Gluconate dehydratase polypeptide of the disclosure is represented by SEQ ID NO: 1:

MSQTPRKLRSQKWFDDPAHADMTAIYVERYLNYGLTRQELQSGRPIIGIA
QTGSDLAPCNRHHLALAERIKAGIRDAGGIPMEFPVHPLAEQGRRPTAAL
DRNLAYLGLVEILHGYPLDGVVLTTGCDKTTPACLMAAATVDIPAIVLSG
GPMLDGWHDGQRVGSGTVIWHARNLMAAGKLDYEGFMTLATASSPSVGHC
NTMGTALSMNSLAEALGMSLPTCASIPAPYRERGQMAYATGLRICDMVRE
DLRPSHILTRQAFENAIVVASALGASSNCPPHLIAMARHAGIDLSLDDWQ
RLGEDVPLLVNCVPAGEHLGEGFHRAGGVPAVLHELAAAGRLHTDCGTVS
GKTIGEIAATAKTNNADVIRSCDAPLRHRAGFIVLSGNFFDSAIIKMSVV
GEAFRRAYLSEPGSENAFEARAIVFEGPEDYHARIEDPSLNIDEHCILVI
RGAGTVGYPGSAEVVNMAPPSHLLKRGIDSLPCLGDGRQSGTSASPSILN
MSPEAAVGGGLALLRTGDRIRVDLNQRSVTALVDETEMERRKLEPPYQAP
ESQTPWQELYRQLVGQLSTGGCLEPATLYLKVVETRGDPRHSH (SEQ I
D NO: 1)

An example of an algorithm that is suitable for determining sequence similarity is the BLAST algorithm, which is described in Altschul et al., 1990, J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, 1992, Proc. Nat′l. Acad. Sci. USA 89:10915-10919) alignments (B) of 50, expectation (E) of 10, M′5, N′-4, and a comparison of both strands.

Any of the amino acid sequences described herein can be produced together or in conjunction with at least 1, e.g., at least (or up to) 2, 3, 5, 10, or 20 heterologous amino acids flanking each of the C- and/or N-terminal ends of the specified amino acid sequence, and or deletions of at least 1, e.g., at least (or up to) 2, 3, 5, 10, or 20 amino acids from the C- and/or N-terminal ends.

Gluconate dehydratase polypeptides of the disclosure may have one or more (e.g., up to 2, 3, 5, 10, 20, 30, 40, or 50) conservative amino acid substitutions relative to the polypeptide of SEQ ID NO: 1. Conservative substitutions can be chosen from among a group of amino acids having a similar side chain to the reference amino acid. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Accordingly, exemplary conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

The disclosure also provides a fusion protein that includes at least a portion (e.g., a fragment or domain) of a gluconate dehydratase polypeptide of the disclosure attached to one or more fusion segments, which are typically heterologous to the gluconate dehydratase polypeptide. Suitable fusion segments include, without limitation, segments that can provide other desirable biological activity like substrate channeling, cellular location, or facilitate purification of the gluconate dehydratase polypeptide (e.g., by affinity chromatography). Fusion segments can be joined to the amino or carboxy terminus of a gluconate dehydratase polypeptide. The fusion segments can be susceptible to cleavage.

### Gluconate Dehydratase Nucleic Acids

A gluconate dehydratase nucleic acid of the disclosure may be isolated, recombinant or synthetic.

In some embodiments, the nucleic acid of the disclosure has at least 70% sequence identity to SEQ ID NO: 2. In some embodiments, the nucleic acid of the disclosure has at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 2, over a region of at least about 10, e.g., at least about 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750 or 1782 nucleotides, or a catalytic domain thereof.

SEQ ID NO: 2 represents the native Achromobacter sp. K91 gluconate dehydratase nucleic acid sequence (locus tag: DXK93_26210). SEQ ID NO: 2 encodes the gluconate dehydratase polypeptide of SEQ ID NO: 1.

ATGTCCCAGACTCCCCGCAAGCTGCGCAGCCAGAAGTGGTTCGACGATCC
GGCCCACGCCGACATGACGGCCATCTATGTCGAGCGCTACCTCAACTACG
GCCTGACGCGCCAGGAACTGCAATCTGGGCGGCCGATCATCGGCATCGCT
CAGACCGGCAGCGACCTGGCGCCCTGCAACCGGCATCACCTGGCGCTGGC
CGAGCGCATCAAGGCCGGCATCCGCGACGCGGGCGGCATCCCCATGGAGT
TTCCCGTGCACCCGCTGGCCGAACAGGGCCGGCGCCCCACCGCCGCGCTG
GACCGCAACCTGGCCTATCTGGGCCTGGTAGAGATCCTGCACGGCTATCC
GCTGGACGGCGTGGTCCTGACCACCGGCTGCGACAAGACCACGCCCGCCT
GCCTCATGGCCGCGGCCACCGTGGATATTCCCGCCATCGTGCTGTCCGGC
GGGCCGATGCTGGACGGCTGGCACGACGGCCAGCGCGTCGGCTCCGGCAC
GGTCATCTGGCATGCGCGCAATCTGATGGCCGCGGGCAAGCTGGACTACG
AAGGCTTCATGACGCTGGCCACGGCGTCGTCGCCGTCCGTCGGCCACTGC
AACACCATGGGCACGGCGCTGTCGATGAACTCGCTGGCCGAAGCGCTGGG
CATGTCGCTGCCCACCTGCGCCAGCATTCCCGCGCCGTACCGCGAGCGCG
GCCAGATGGCCTACGCCACCGGGCTGCGTATCTGCGACATGGTGCGCGAG
GACTTGCGCCCGTCCCACATCCTGACGCGGCAGGCTTTCGAGAACGCCAT
CGTCGTGGCCTCGGCGCTGGGCGCCTCCAGCAACTGCCCGCCGCACCTCA
TCGCCATGGCGCGCCACGCCGGCATCGACCTGAGCCTGGACGACTGGCAG
CGCTTGGGCGAAGACGTGCCGCTGCTGGTCAACTGTGTACCTGCGGGCGA
GCATCTGGGCGAAGGCTTTCACCGCGCGGGCGGCGTCCCGGCGGTGTTGC
ACGAACTGGCGGCAGCAGGCCGCCTGCATACCGATTGCGGCACCGTGTCC
GGCAAGACCATCGGCGAGATCGCGGCTACGGCCAAGACCAACAATGCGGA
CGTCATCCGCAGCTGCGACGCGCCGCTCAGGCATCGCGCGGGATTCATCG
TCCTCTCGGGCAACTTCTTCGACAGCGCCATCATCAAGATGTCGGTCGTC
GGCGAAGCCTTCCGCCGCGCCTATCTGTCGGAGCCCGGTTCAGAGAACGC
CTTCGAGGCGCGCGCCATCGTCTTCGAAGGCCCCGAGGACTACCACGCGC
GCATCGAAGACCCCTCCCTGAACATCGACGAGCACTGCATCCTGGTGATC
CGCGGCGCCGGCACCGTCGGCTACCCCGGCAGCGCGGAAGTGGTCAACAT
GGCGCCGCCCTCGCATCTGCTCAAGCGCGGCATAGACTCTCTGCCCTGCC
TGGGCGACGGCCGCCAGAGCGGCACCTCGGCCAGCCCTTCGATCCTGAAC
ATGTCGCCGGAAGCCGCGGTGGGCGGTGGCCTCGCGCTGTTGCGCACGGG
CGACCGCATCCGCGTCGACCTGAATCAGCGCTCGGTCACCGCCCTGGTGG
ATGAAACAGAAATGGAACGCCGCAAGTTGGAGCCGCCCTACCAGGCGCCG
GAATCTCAGACGCCGTGGCAAGAGCTGTACCGGCAACTGGTCGGCCAGCT
TTCAACCGGCGGCTGCCTGGAGCCCGCCACCTTGTATCTGAAGGTCGTGG
AAACACGGGGCGATCCCCGGCATTCGCATTGA (SEQ ID NO:2)

In some embodiments, the nucleic acid has at least 70% sequence identity to SEQ ID NO: 3. In some embodiments, the nucleic acid sequence has at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 3, over a region of at least about 10, e.g., at least about 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750 or 1782 nucleotides, or a catalytic domain thereof.

SEQ ID NO: 3 represents the native Achromobacter sp. 2789STDY5608625 gluconate dehydratase nucleic acid sequence (locus tag: AOZ63_RS28315). SEQ ID NO: 3 encodes the gluconate dehydratase polypeptide sequence of SEQ ID NO: 1.

ATGTCCCAGACTCCCCGCAAGCTGCGCAGCCAGAAGTGGTTCGACGATCC
GGCCCACGCCGACATGACGGCCATCTATGTCGAGCGCTACCTCAACTACG
GCCTGACGCGCCAGGAACTGCAATCTGGGCGGCCGATCATCGGCATCGCT
CAGACCGGCAGCGACCTGGCGCCCTGCAACCGGCATCACCTGGCGCTGGC
CGAGCGCATCAAGGCCGGCATCCGCGACGCGGGCGGCATCCCCATGGAGT
TTCCCGTGCACCCGCTGGCCGAACAGGGCCGGCGCCCCACCGCCGCGCTG
GACCGCAACCTGGCCTATCTGGGCCTGGTAGAGATCCTGCACGGCTATCC
GCTGGACGGCGTGGTCCTGACCACCGGCTGCGACAAGACCACGCCCGCCT
GCCTCATGGCCGCGGCCACCGTGGATATTCCCGCCATCGTGCTGTCCGGC
GGGCCGATGCTGGACGGCTGGCACGACGGCCAGCGCGTCGGCTCCGGCAC
GGTCATCTGGCATGCGCGCAATCTGATGGCCGCGGGCAAGCTGGACTACG
AAGGCTTCATGACGCTGGCCACGGCGTCGTCGCCGTCCGTCGGCCACTGC
AACACCATGGGCACGGCGCTGTCGATGAACTCGCTGGCCGAAGCGCTGGG
CATGTCGCTGCCCACCTGCGCCAGCATTCCCGCGCCGTACCGCGAGCGCG
GCCAGATGGCCTACGCCACCGGGCTGCGTATCTGCGACATGGTGCGCGAG
GACTTGCGCCCATCCCATATCCTGACGCGGCAGGCTTTCGAGAACGCCAT
CGTCGTGGCCTCGGCGCTGGGCGCCTCCAGCAACTGCCCGCCGCACCTCA
TCGCCATGGCCCGCCACGCCGGCATCGACCTGAGCCTGGACGACTGGCAG
CGCTTGGGCGAAGACGTGCCGCTGCTGGTCAACTGTGTACCTGCGGGTGA
GCATCTGGGCGAAGGCTTTCACCGCGCGGGCGGCGTCCCGGCGGTGTTGC
ACGAACTGGCGGCAGCAGGCCGCCTGCATACCGATTGCGGCACCGTGTCC
GGCAAGACCATCGGCGAGATCGCGGCTACGGCCAAGACCAACAATGCGGA
CGTCATCCGCAGCTGCGACGCGCCGCTCAGGCATCGCGCGGGATTCATCG
TCCTCTCGGGCAACTTCTTCGACAGCGCCATCATCAAGATGTCGGTCGTC
GGCGAAGCCTTCCGCCGCGCCTATCTGTCGGAGCCCGGTTCAGAGAACGC
CTTCGAGGCGCGCGCCATCGTCTTCGAAGGCCCCGAGGACTACCACGCGC
GCATCGAAGACCCCTCCCTGAACATCGACGAGCACTGCATCCTGGTGATC
CGCGGCGCCGGCACCGTCGGCTACCCCGGCAGCGCGGAAGTGGTCAACAT
GGCGCCGCCCTCGCATCTGCTCAAGCGCGGCATAGACTCTCTGCCCTGCC
TGGGCGACGGCCGCCAGAGCGGCACCTCGGCCAGCCCTTCGATCCTGAAC
ATGTCGCCGGAAGCCGCGGTGGGCGGTGGCCTCGCGCTGTTGCGCACGGG
CGACCGCATCCGCGTCGACCTGAACCAGCGCTCGGTCACCGCCCTGGTGG
ATGAAACAGAAATGGAACGCCGCAAGCTGGAGCCGCCCTACCAGGCGCCG
GAATCTCAGACGCCGTGGCAAGAGCTATACCGGCAACTGGTCGGCCAGCT
TTCAACCGGCGGCTGCCTGGAGCCCGCCACCTTGTATCTGAAGGTCGTGG
AAACGCGGGGCGATCCCCGGCATTCGCATTGA (SEQ ID NO:3)

A gluconate dehydratase nucleic acid of the disclosure may be optimized for expression in a recombinant cell. “Optimized” nucleic acid sequences encode an amino acid sequence using codons that are preferred in a recombinant cell. The optimized nucleic acid sequence is typically engineered to retain completely or as much as possible of the amino acid sequence originally encoded by the starting nucleic acid sequence, which is also known as the “parental” sequence. Several methods for codon optimization are known in the art. Preferably, codon optimized sequences avoid nucleotide repeats and restriction sites that are utilized in cloning the gluconate dehydratase nucleic acids, by adjusting the settings in commercial software or by manually altering the sequences to substitute codons that introduce undesired sequences, for example with highly utilized codons in the heterologous organism of interest.

In some embodiments, the nucleic acid is codon optimized for expression in a prokaryotic cell. In some embodiments, the nucleic acid is codon optimized for expression in Acinetobacter, Agrobacterium, Escherichia, Cupriavidus, Clostridium, Rhodobacter, Marinobacter, Bacillus, Klebsiella, Tatumella, Pseudomonas, Ralstonia, Rhodococcus, Methylobacterium, Methylophilus, Methylococcus, Methylomicrobium, Methylomonas, Pantoea, Streptomyces, Parachlorella, Synechococcus, Synechocystis and Thermocynechococcus. In some embodiments, the nucleic acid is codon optimized for expression in E. coli.

The disclosure also provides a nucleic acid comprising a sequence having at least 70% sequence identity to SEQ ID NO: 4. In some embodiments, the nucleic acid sequence has at least 75%, at least 80%, at least 85%, at least 90%, at least 95%, at least 96%, at least 97%, at least 98%, or at least 99%, or 100% sequence identity to SEQ ID NO: 4, over a region of at least about 10, e.g., at least about 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750 or 1782 nucleotides, or a catalytic domain thereof.

SEQ ID NO: 4 represents a nucleic acid sequence encoding the gluconate dehydratase polypeptide of SEQ ID NO: 1, which has been codon optimized for expression in E. coli:

ATGTCGCAAACTCCTCGCAAACTGCGTTCGCAGAAGTGGTTTGACGATCC
GGCACATGCAGATATGACGGCCATCTATGTCGAACGTTACTTGAACTACG
GGTTGACCCGTCAAGAGCTGCAAAGTGGCCGCCCGATCATTGGAATTGCC
CAAACTGGGTCCGATTTAGCTCCTTGTAACCGCCATCATCTTGCACTTGC
TGAGCGTATCAAGGCCGGAATTCGTGACGCCGGTGGCATCCCGATGGAAT
TTCCTGTGCATCCCTTGGCGGAACAGGGCCGTCGTCCAACTGCGGCCTTA
GACCGCAACTTAGCATATCTTGGGCTGGTCGAAATCCTGCACGGGTATCC
CTTGGACGGAGTTGTGCTGACGACAGGTTGCGACAAGACCACCCCTGCTT
GTCTGATGGCAGCTGCAACAGTGGACATCCCGGCGATTGTGCTGAGCGGC
GGACCCATGCTTGACGGGTGGCACGACGGCCAGCGCGTGGGCAGTGGAAC
AGTGATCTGGCACGCGCGTAATTTAATGGCAGCGGGTAAATTAGATTACG
AAGGTTTTATGACACTGGCCACTGCTAGCAGTCCCTCCGTCGGTCATTGC
AACACAATGGGGACGGCACTTAGCATGAACTCGCTGGCAGAAGCTCTGGG
AATGTCCTTACCGACATGCGCATCAATCCCGGCCCCCTATCGCGAACGTG
GGCAAATGGCATACGCGACTGGTTTGCGCATCTGCGACATGGTACGTGAG
GATCTTCGTCCCTCGCACATCCTTACTCGTCAGGCCTTTGAAAACGCGAT
TGTCGTTGCCTCAGCTTTGGGCGCGAGTAGTAACTGCCCACCTCATTTGA
TTGCGATGGCCCGTCACGCCGGAATCGATTTATCGCTTGATGATTGGCAA
CGCCTTGGAGAAGATGTTCCGTTGCTTGTCAATTGCGTTCCCGCGGGTGA
GCATCTTGGGGAGGGATTCCACCGTGCCGGGGGCGTACCTGCCGTTCTTC
ACGAATTGGCAGCGGCTGGGCGCCTTCACACCGACTGCGGAACCGTTTCC
GGGAAAACGATCGGCGAGATCGCAGCAACAGCCAAGACTAATAACGCAGA
TGTAATCCGTTCTTGTGATGCTCCCCTTCGTCACCGCGCCGGGTTCATCG
TATTATCAGGGAACTTTTTTGACTCCGCCATCATCAAAATGTCGGTCGTA
GGCGAGGCATTCCGCCGCGCATACTTGTCGGAACCGGGTTCAGAAAATGC
GTTCGAAGCTCGTGCGATTGTGTTTGAGGGCCCAGAAGACTACCATGCGC
GCATTGAAGATCCTTCTCTTAATATTGACGAACATTGTATCCTGGTAATC
CGCGGAGCGGGGACGGTGGGTTACCCAGGTTCGGCGGAAGTAGTCAATAT
GGCTCCTCCCAGTCACCTTTTAAAACGCGGTATTGACTCATTACCGTGTT
TAGGGGATGGTCGCCAGAGCGGAACGAGTGCATCTCCCAGCATCTTGAAT
ATGTCTCCAGAGGCCGCGGTCGGGGGAGGCTTGGCGCTTCTGCGCACCGG
TGACCGCATTCGTGTTGACCTGAACCAGCGCTCAGTTACGGCGCTTGTCG
ATGAGACTGAAATGGAGCGTCGTAAATTGGAACCACCTTATCAGGCCCCA
GAATCGCAGACCCCGTGGCAAGAGTTGTATCGCCAGTTAGTTGGGCAATT
GTCCACGGGAGGATGTCTTGAGCCCGCGACATTGTACCTTAAGGTCGTAG
AGACACGTGGTGATCCGCGTCACTCTCACTAA (SEQ ID NO:4)

In some embodiments, the nucleic acid is codon optimized for expression in a eukaryotic cell. In some embodiments, the nucleic acid is codon optimized for expression in a yeast cell, a fungal cell, or an algal cell. In some embodiments, the nucleic acid is codon optimized for expression in Saccharomyces, Kluyveromyces, Candida, Pichia, Schizosaccharomyces, Schefferomyces, Rhodosporidium, Hansenula, Klockera, Schwanniomyces, Issatchenkia, Yarrowia or Rhodotorula. In some embodiments, the nucleic acid is codon optimized for expression in S. cerevisiae, C. lipolytica, R. glutinis, S. bulderi, S. barnetti, S. exiguus, S. uvarum, S. diastaticus, K. lactis, K. marxianus K. fragile, P. kudriavzevii, S. stipitis or I. orientalis.

In some embodiments, the nucleic acid is codon optimized for expression in a filamentous fungal cell. In some embodiments, the nucleic acid is codon optimized for expression in Aspergillus, Penicillium, Rhizopus, Chrysosporium, Myceliophthora, Trichoderma, Humicola, Acremonium or Fusarium. In some embodiments, the nucleic acid is codon optimized for expression in A. niger, A. oryzae, T. reesei, P. chrysogenum, M. thermophila, or R. oryzae.

In some embodiments, the nucleic acid is codon optimized for expression in Botryococcus, Nannochloropsis, Chlorella, Chlamydomonas, Dunaliella, Chaetoceros, Porphyridium, Scenedesmus or Pseudochlorococcum. In some embodiments, the nucleic acid is codon optimized for expression in B. braunii or N. gaditana.

### Vectors

A vector of the disclosure comprises a gluconate dehydratase nucleic acid of the disclosure. A vector may comprise one or more of an origin of replication, a promoter sequence operably linked to a nucleic acid of the disclosure and a reporter gene or selectable marker.

Vectors according to the disclosure include, but are not limited to, pTrc-His2C, pCL-BP2, pBR322, pBR327, pACYC184, pACYC177, pSC101, pCL1920, and pCL1921.

Vectors of the disclosure comprising an origin of replication include, but are not limited to, ColE1, pMB1, p15A, pSC101, R6K, RK2 and pRSF1010.

The promoter may be homologous or heterologous. The promoter may be constitutive or inducible.

In certain embodiments, the promoter is inducible and is activated in the presence of an inducing agent. Inducing agents include, but are not limited to, sugars, metal salts, and antibiotics. In some cases, the promoter allows constitutive expression of the gluconate dehydratase polypeptide.

In certain embodiments, promoters that are active at different stages of growth can be used.

The promoter sequence may be operable in a prokaryotic cell, for example an E. coli cell. In certain embodiments the promoter is a Trc promoter.

The promoter sequence may be operable in a eukaryotic cell, for example a yeast cell, a fungal cell, or an algal cell.

Where the recombinant cell is a fungal cell, the promoter can be a fungal promoter (including, but not limited to, a filamentous fungal promoter), a promoter operable in plant cells, or a promoter operable in mammalian cells. Mammalian, mammalian viral, plant and plant viral promoters can drive particularly high expression when the associated 5′ UTR sequence (i.e., the sequence which begins at the transcription start site and ends one nucleotide before the start codon), normally associated with the mammalian or mammalian viral promoter is replaced by a fungal 5′ UTR sequence. The source of the 5′ UTR can vary provided it is operable in the filamentous fungal cell. In various embodiments, the 5′ UTR can be derived from a yeast gene or a filamentous fungal gene. The 5′ UTR can be from the same species as the recombinant cell or from a different species.

Promoters for recombinant expression in yeast are known in the art. Suitable promoters for S. cerevisiae include, but are not limited to, the MFα1 promoter, galactose inducible promoters such as GAL1, GAL7, and GAL10 promoters, glycolytic enzyme promoters including the TPI and PGK promoters, the TDH3 promoter, the TEF1 promoter, the TRP1 promoter, the CYCI promoter, the CUP1 promoter, the PHO5 promoter, the ADH1 promoter, and the HDP promoter. A suitable promoter in the genus Pichia sp. is the AOXI promoter.

The terms “reporter gene” and “selectable marker” are used interchangeably herein. Suitable reporter genes or selectable markers include, but are not limited to, a drug resistance gene, a metabolic enzyme, a factor required for survival of the recombinant cell, a fluorescent marker, or an enzyme that generates a detectable product. Cells transformed with the vector can be selected based on their ability to grow in the presence of inhibitors (e.g. antibiotics) or under conditions in which untransformed cells cannot grow.

The vector may be a high copy number vector, an intermediate copy number vector, or a low copy number vector. Preferably, the vector is a low copy number vector, e.g. vector pCL-BP2.

In some embodiments, the vector comprises a nucleic acid sequence having at least 70% sequence identity to SEQ ID NO: 5. In some embodiments, the vector comprises a nucleic acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 5. Vector corresponding to SEQ ID NO: 5 is also referred to herein as pTrc-His2C.

GTTTGACAGCTTATCATCGACTGCACGGTGCACCAATGCTTCTGGCGTCA
GGCAGCCATCGGAAGCTGTGGTATGGCTGTGCAGGTCGTAAATCACTGCA
TAATTCGTGTCGCTCAAGGCGCACTCCCGTTCTGGATAATGTTTTTTGCG
CCGACATCATAACGGTTCTGGCAAATATTCTGAAATGAGCTGTTGACAAT
TAATCATCCGGCTCGTATAATGTGTGGAATTGTGAGCGGATAACAATTTC
ACACAGGAAACAGCGCCGCTGAGAAAAAGCGAAGCGGCACTGCTCTTTAA
CAATTTATCAGACAATCTGTGTGGGCACTCGACCGGAATTATCGATTAAC
TTTATTATTAAAAATTAAAGAGGTATATATTAATGTATCGATTAAATAAG
GAGGAATAAACCGCTTTCTAGAACAAAAACTCATCTCAGAAGAGGATCTG
AATAGCGCCGTCGACCATCATCATCATCATCATTGAGTTTAAACGGTCTC
CAGCTTGGCTGTTTTGGCGGATGAGAGAAGATTTTCAGCCTGATACAGAT
TAAATCAGAACGCAGAAGCGGTCTGATAAAACAGAATTTGCCTGGCGGCA
GTAGCGCGGTGGTCCCACCTGACCCCATGCCGAACTCAGAAGTGAAACGC
CGTAGCGCCGATGGTAGTGTGGGGTCTCCCCATGCGAGAGTAGGGAACTG
CCAGGCATCAAATAAAACGAAAGGCTCAGTCGAAAGACTGGGCCTTTCGT
TTTATCTGTTGTTTGTCGGTGAACGCTCTCCTGAGTAGGACAAATCCGCC
GGGAGCGGATTTGAACGTTGCGAAGCAACGGCCCGGAGGGTGGCGGGCAG
GACGCCCGCCATAAACTGCCAGGCATCAAATTAAGCAGAAGGCCATCCTG
ACGGATGGCCTTTTTGCGTTTCTACAAACTCTTTTTGTTTATTTTTCTAA
ATACATTCAAATATGTATCCGCTCATGAGACAATAACCCTGATAAATGCT
TCAATAATATTGAAAAAGGAAGAGTATGAGTATTCAACATTTCCGTGTCG
CCCTTATTCCCTTTTTTGCGGCATTTTGCCTTCCTGTTTTTGCTCACCCA
GAAACGCTGGTGAAAGTAAAAGATGCTGAAGATCAGTTGGGTGCACGAGT
GGGTTACATCGAACTGGATCTCAACAGCGGTAAGATCCTTGAGAGTTTTC
GCCCCGAAGAACGTTTTCCAATGATGAGCACTTTTAAAGTTCTGCTATGT
GGCGCGGTATTATCCCGTGTTGACGCCGGGCAAGAGCAACTCGGTCGCCG
CATACACTATTCTCAGAATGACTTGGTTGAGTACTCACCAGTCACAGAAA
AGCATCTTACGGATGGCATGACAGTAAGAGAATTATGCAGTGCTGCCATA
ACCATGAGTGATAACACTGCGGCCAACTTACTTCTGACAACGATCGGAGG
ACCGAAGGAGCTAACCGCTTTTTTGCACAACATGGGGGATCATGTAACTC
GCCTTGATCGTTGGGAACCGGAGCTGAATGAAGCCATACCAAACGACGAG
CGTGACACCACGATGCCTGTAGCAATGGCAACAACGTTGCGCAAACTATT
AACTGGCGAACTACTTACTCTAGCTTCCCGGCAACAATTAATAGACTGGA
TGGAGGCGGATAAAGTTGCAGGACCACTTCTGCGCTCGGCCCTTCCGGCT
GGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTA
TCTACACGACGGGGAGTCAGGCAACTATGGATGAACGAAATAGACAGATC
GCTGAGATAGGTGCCTCACTGATTAAGCATTGGTAACTGTCAGACCAAGT
TTACTCATATATACTTTAGATTGATTTAAAACTTCATTTTTAATTTAAAA
GGATCTAGGTGAAGATCCTTTTTGATAATCTCATGACCAAAATCCCTTAA
CGTGAGTTTTCGTTCCACTGAGCGTCAGACCCCGTAGAAAAGATCAAAGG
ATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCA
ACTCTTTTTCCGAAGGTAACTGGCTTCAGCAGAGCGCAGATACCAAATAC
TGTCCTTCTAGTGTAGCCGTAGTTAGGCCACCACTTCAAGAACTCTGTAG
CACCGCCTACATACCTCGCTCTGCTAATCCTGTTACCAGTGGCTGCTGCC
AGTGGCGATAAGTCGTGTCTTACCGGGTTGGACTCAAGACGATAGTTACC
GGATAAGGCGCAGCGGTCGGGCTGAACGGGGGGTTCGTGCACACAGCCCA
GCTTGGAGCGAACGACCTACACCGAACTGAGATACCTACAGCGTGAGCTA
TGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGC
CAGCAACGCGGCCTTTTTACGGTTCCTGGCCTTTTGCTGGCCTTTTGCTC
ACATGTTCTTTCCTGCGTTATCCCCTGATTCTGTGGATAACCGTATTACC
GCCTTTGAGTGAGCTGATACCGCTCGCCGCAGCCGAACGACCGAGCGCAG
CGAGTCAGTGAGCGAGGAAGCGGAAGAGCGCCTGATGCGGTATTTTCTCC
TTACGCATCTGTGCGGTATTTCACACCGCATATGGTGCACTCTCAGTACA
ATCTGCTCTGATGCCGCATAGTTAAGCCAGTATACACTCCGCTATCGCTA
CGTGACTGGGTCATGGCTGCGCCCCGACACCCGCCAACACCCGCTGACGC
GCCCTGACGGGCTTGTCTGCTCCCGGCATCCGCTTACAGACAAGCTGTGA
CCGTCTCCGGGAGCTGCATGTGTCAGAGGTTTTCACCGTCATCACCGAAA
CGCGCGAGGCAGCAGATCAATTCGCGCGCGAAGGCGAAGCGGCATGCATT
TACGTTGACACCATCGAATGGTGCAAAACCTTTCGCGGTATGGCATGATA
GCGCCCGGAAGAGAGTCAATTCAGGGTGGTGAATGTGAAACCAGTAACGT
TATACGATGTCGCAGAGTATGCCGGTGTCTCTTATCAGACCGTTTCCCGC
GTGGTGAACCAGGCCAGCCACGTTTCTGCGAAAACGCGGGAAAAAGTGGA
AGCGGCGATGGCGGAGCTGAATTACATTCCCAACCGCGTGGCACAACAAC
TGGCGGGCAAACAGTCGTTGCTGATTGGCGTTGCCACCTCCAGTCTGGCC
CTGCACGCGCCGTCGCAAATTGTCGCGGCGATTAAATCTCGCGCCGATCA
ACTGGGTGCCAGCGTGGTGGTGTCGATGGTAGAACGAAGCGGCGTCGAAG
CCTGTAAAGCGGCGGTGCACAATCTTCTCGCGCAACGCGTCAGTGGGCTG
ATCATTAACTATCCGCTGGATGACCAGGATGCCATTGCTGTGGAAGCTGC
CTGCACTAATGTTCCGGCGTTATTTCTTGATGTCTCTGACCAGACACCCA
TCAACAGTATTATTTTCTCCCATGAAGACGGTACGCGACTGGGCGTGGAG
CATCTGGTCGCATTGGGTCACCAGCAAATCGCGCTGTTAGCGGGCCCATT
AAGTTCTGTCTCGGCGCGTCTGCGTCTGGCTGGCTGGCATAAATATCTCA
CTCGCAATCAAATTCAGCCGATAGCGGAACGGGAAGGCGACTGGAGTGCC
ATGTCCGGTTTTCAACAAACCATGCAAATGCTGAATGAGGGCATCGTTCC
CACTGCGATGCTGGTTGCCAACGATCAGATGGCGCTGGGCGCAATGCGCG
CCATTACCGAGTCCGGGCTGCGCGTTGGTGCGGATATCTCGGTAGTGGGA
TACGACGATACCGAAGACAGCTCATGTTATATCCCGCCGTCAACCACCAT
CAAACAGGATTTTCGCCTGCTGGGGCAAACCAGCGTGGACCGCTTGCTGC
AACTCTCTCAGGGCCAGGCGGTGAAGGGCAATCAGCTGTTGCCCGTCTCA
CTGGTGAAAAGAAAAACCACCCTGGCGCCCAATACGCAAACCGCCTCTCC
CCGCGCGTTGGCCGATTCATTAATGCAGCTGGCACGACAGGTTTCCCGAC
TGGAAAGCGGGCAGTGAGCGCAACGCAATTAATGTGAGTTAGCGCGAATT
GATCTG (SEQ ID NO: 5)

In some embodiments, the vector comprises a nucleic acid sequence having at least 70% sequence identity to SEQ ID NO: 6. The vector of SEQ ID NO: 6 corresponds to the vector of SEQ ID NO: 5 comprising the nucleic acid of SEQ ID NO: 4. In some embodiments, the vector comprises a nucleic acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 6. Vector corresponding to SEQ ID NO: 6 is also referred to herein as pAchro-10.

GTTTGACAGCTTATCATCGACTGCACGGTGCACCAATGCTTCTGGCGTCA
GGCAGCCATCGGAAGCTGTGGTATGGCTGTGCAGGTCGTAAATCACTGCA
TAATTCGTGTCGCTCAAGGCGCACTCCCGTTCTGGATAATGTTTTTTGCG
CCGACATCATAACGGTTCTGGCAAATATTCTGAAATGAGCTGTTGACAAT
TAATCATCCGGCTCGTATAATGTGTGGAATTGTGAGCGGATAACAATTTC
ACACAGGAAACAGCGCCGCTGAGAAAAAGCGAAGCGGCACTGCTCTTTAA
CAATTTATCAGACAATCTGTGTGGGCACTCGACCGGAATTATCGATTAAC
TTTATTATTAAAAATTAAAGAGGTATATATTAATGTATCGATTAAATAAG
GAGGAATAAACCATGTCGCAAACTCCTCGCAAACTGCGTTCGCAGAAGTG
GTTTGACGATCCGGCACATGCAGATATGACGGCCATCTATGTCGAACGTT
ACTTGAACTACGGGTTGACCCGTCAAGAGCTGCAAAGTGGCCGCCCGATC
ATTGGAATTGCCCAAACTGGGTCCGATTTAGCTCCTTGTAACCGCCATCA
TCTTGCACTTGCTGAGCGTATCAAGGCCGGAATTCGTGACGCCGGTGGCA
TCCCGATGGAATTTCCTGTGCATCCCTTGGCGGAACAGGGCCGTCGTCCA
ACTGCGGCCTTAGACCGCAACTTAGCATATCTTGGGCTGGTCGAAATCCT
GCACGGGTATCCCTTGGACGGAGTTGTGCTGACGACAGGTTGCGACAAGA
CCACCCCTGCTTGTCTGATGGCAGCTGCAACAGTGGACATCCCGGCGATT
GTGCTGAGCGGCGGACCCATGCTTGACGGGTGGCACGACGGCCAGCGCGT
GGGCAGTGGAACAGTGATCTGGCACGCGCGTAATTTAATGGCAGCGGGTA
AATTAGATTACGAAGGTTTTATGACACTGGCCACTGCTAGCAGTCCCTCC
GTCGGTCATTGCAACACAATGGGGACGGCACTTAGCATGAACTCGCTGGC
AGAAGCTCTGGGAATGTCCTTACCGACATGCGCATCAATCCCGGCCCCCT
ATCGCGAACGTGGGCAAATGGCATACGCGACTGGTTTGCGCATCTGCGAC
ATGGTACGTGAGGATCTTCGTCCCTCGCACATCCTTACTCGTCAGGCCTT
TGAAAACGCGATTGTCGTTGCCTCAGCTTTGGGCGCGAGTAGTAACTGCC
CACCTCATTTGATTGCGATGGCCCGTCACGCCGGAATCGATTTATCGCTT
GATGATTGGCAACGCCTTGGAGAAGATGTTCCGTTGCTTGTCAATTGCGT
TCCCGCGGGTGAGCATCTTGGGGAGGGATTCCACCGTGCCGGGGGCGTAC
CTGCCGTTCTTCACGAATTGGCAGCGGCTGGGCGCCTTCACACCGACTGC
GGAACCGTTTCCGGGAAAACGATCGGCGAGATCGCAGCAACAGCCAAGAC
TAATAACGCAGATGTAATCCGTTCTTGTGATGCTCCCCTTCGTCACCGCG
CCGGGTTCATCGTATTATCAGGGAACTTTTTTGACTCCGCCATCATCAAA
ATGTCGGTCGTAGGCGAGGCATTCCGCCGCGCATACTTGTCGGAACCGGG
TTCAGAAAATGCGTTCGAAGCTCGTGCGATTGTGTTTGAGGGCCCAGAAG
ACTACCATGCGCGCATTGAAGATCCTTCTCTTAATATTGACGAACATTGT
ATCCTGGTAATCCGCGGAGCGGGGACGGTGGGTTACCCAGGTTCGGCGGA
AGTAGTCAATATGGCTCCTCCCAGTCACCTTTTAAAACGCGGTATTGACT
CATTACCGTGTTTAGGGGATGGTCGCCAGAGCGGAACGAGTGCATCTCCC
AGCATCTTGAATATGTCTCCAGAGGCCGCGGTCGGGGGAGGCTTGGCGCT
TCTGCGCACCGGTGACCGCATTCGTGTTGACCTGAACCAGCGCTCAGTTA
CGGCGCTTGTCGATGAGACTGAAATGGAGCGTCGTAAATTGGAACCACCT
TATCAGGCCCCAGAATCGCAGACCCCGTGGCAAGAGTTGTATCGCCAGTT
AGTTGGGCAATTGTCCACGGGAGGATGTCTTGAGCCCGCGACATTGTACC
TTAAGGTCGTAGAGACACGTGGTGATCCGCGTCACTCTCACTAAGCTTTC
TAGAACAAAAACTCATCTCAGAAGAGGATCTGAATAGCGCCGTCGACCAT
CATCATCATCATCATTGAGTTTAAACGGTCTCCAGCTTGGCTGTTTTGGC
GGATGAGAGAAGATTTTCAGCCTGATACAGATTAAATCAGAACGCAGAAG
CGGTCTGATAAAACAGAATTTGCCTGGCGGCAGTAGCGCGGTGGTCCCAC
CTGACCCCATGCCGAACTCAGAAGTGAAACGCCGTAGCGCCGATGGTAGT
GTGGGGTCTCCCCATGCGAGAGTAGGGAACTGCCAGGCATCAAATAAAAC
GAAAGGCTCAGTCGAAAGACTGGGCCTTTCGTTTTATCTGTTGTTTGTCG
GTGAACGCTCTCCTGAGTAGGACAAATCCGCCGGGAGCGGATTTGAACGT
TGCGAAGCAACGGCCCGGAGGGTGGCGGGCAGGACGCCCGCCATAAACTG
CCAGGCATCAAATTAAGCAGAAGGCCATCCTGACGGATGGCCTTTTTGCG
TTTCTACAAACTCTTTTTGTTTATTTTTCTAAATACATTCAAATATGTAT
CCGCTCATGAGACAATAACCCTGATAAATGCTTCAATAATATTGAAAAAG
GAAGAGTATGAGTATTCAACATTTCCGTGTCGCCCTTATTCCCTTTTTTG
CGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGA
TCTCAACAGCGGTAAGATCCTTGAGAGTTTTCGCCCCGAAGAACGTTTTC
CAATGATGAGCACTTTTAAAGTTCTGCTATGTGGCGCGGTATTATCCCGT
GTTGACGCCGGGCAAGAGCAACTCGGTCGCCGCATACACTATTCTCAGAA
TGACTTGGTTGAGTACTCACCAGTCACAGAAAAGCATCTTACGGATGGCA
TGACAGTAAGAGAATTATGCAGTGCTGCCATAACCATGAGTGATAACACT
GCGGCCAACTTACTTCTGACAACGATCGGAGGACCGAAGGAGCTAACCGC
TTTTTTGCACAACATGGGGGATCATGTAACTCGCCTTGATCGTTGGGAAC
CGGAGCTGAATGAAGCCATACCAAACGACGAGCGTGACACCACGATGCCT
GTAGCAATGGCAACAACGTTGCGCAAACTATTAACTGGCGAACTACTTAC
TCTAGCTTCCCGGCAACAATTAATAGACTGGATGGAGGCGGATAAAGTTG
CAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGG
GCCAGATGGTAAGCCCTCCCGTATCGTAGTTATCTACACGACGGGGAGTC
AGGCAACTATGGATGAACGAAATAGACAGATCGCTGAGATAGGTGCCTCA
CTGATTAAGCATTGGTAACTGTCAGACCAAGTTTACTCATATATACTTTA
GATTGATTTAAAACTTCATTTTTAATTTAAAAGGATCTAGGTGAAGATCC
TTTTTGATAATCTCATGACCAAAATCCCTTAACGTGAGTTTTCGTTCCAC
TGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTA
ACTGGCTTCAGCAGAGCGCAGATACCAAATACTGTCCTTCTAGTGTAGCC
GTAGTTAGGCCACCACTTCAAGAACTCTGTAGCACCGCCTACATACCTCG
CTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTC
GGGCTGAACGGGGGGTTCGTGCACACAGCCCAGCTTGGAGCGAACGACCT
ACACCGAACTGAGATACCTACAGCGTGAGCTATGAGAAAGCGCCACGCTT
CCCGAAGGGAGAAAGGCGGACAGGTATCCGGTAAGCGGCAGGGTCGGAAC
AGGAGAGCGCACGAGGGAGCTTCCAGGGGGAAACGCCTGGTATCTTTATA
GTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTTTT
ACGGTTCCTGGCCTTTTGCTGGCCTTTTGCTCACATGTTCTTTCCTGCGT
TATCCCCTGATTCTGTGGATAACCGTATTACCGCCTTTGAGTGAGCTGAT
ACCGCTCGCCGCAGCCGAACGACCGAGCGCAGCGAGTCAGTGAGCGAGGA
AGCGGAAGAGCGCCTGATGCGGTATTTTCTCCTTACGCATCTGTGCGGTA
TTTCACACCGCATATGGTGCACTCTCAGTACAATCTGCTCTGATGCCGCA
TAGTTAAGCCAGTATACACTCCGCTATCGCTACGTGACTGGGTCATGGCT
GCGCCCCGACACCCGCCAACACCCGCTGACGCGCCCTGACGGGCTTGTCT
GCTCCCGGCATCCGCTTACAGACAAGCTGTGACCGTCTCCGGGAGCTGCA
TGTGTCAGAGGTTTTCACCGTCATCACCGAAACGCGCGAGGCAGCAGATC
AATTCGCGCGCGAAGGCGAAGCGGCATGCATTTACGTTGACACCATCGAA
TGGTGCAAAACCTTTCGCGGTATGGCATGATAGCGCCCGGAAGAGAGTCA
ATTCAGGGTGGTGAATGTGAAACCAGTAACGTTATACGATGTCGCAGAGT
ATGCCGGTGTCTCTTATCAGACCGTTTCCCGCGTGGTGAACCAGGCCAGC
CACGTTTCTGCGAAAACGCGGGAAAAAGTGGAAGCGGCGATGGCGGAGCT
GAATTACATTCCCAACCGCGTGGCACAACAACTGGCGGGCAAACAGTCGT
TGCTGATTGGCGTTGCCACCTCCAGTCTGGCCCTGCACGCGCCGTCGCAA
ATTGTCGCGGCGATTAAATCTCGCGCCGATCAACTGGGTGCCAGCGTGGT
GGTGTCGATGGTAGAACGAAGCGGCGTCGAAGCCTGTAAAGCGGCGGTGC
ACAATCTTCTCGCGCAACGCGTCAGTGGGCTGATCATTAACTATCCGCTG
GATGACCAGGATGCCATTGCTGTGGAAGCTGCCTGCACTAATGTTCCGGC
GTTATTTCTTGATGTCTCTGACCAGACACCCATCAACAGTATTATTTTCT
CCCATGAAGACGGTACGCGACTGGGCGTGGAGCATCTGGTCGCATTGGGT
CACCAGCAAATCGCGCTGTTAGCGGGCCCATTAAGTTCTGTCTCGGCGCG
TCTGCGTCTGGCTGGCTGGCATAAATATCTCACTCGCAATCAAATTCAGC
CGATAGCGGAACGGGAAGGCGACTGGAGTGCCATGTCCGGTTTTCAACAA
ACCATGCAAATGCTGAATGAGGGCATCGTTCCCACTGCGATGCTGGTTGC
CAACGATCAGATGGCGCTGGGCGCAATGCGCGCCATTACCGAGTCCGGGC
TGCGCGTTGGTGCGGATATCTCGGTAGTGGGATACGACGATACCGAAGAC
AGCTCATGTTATATCCCGCCGTCAACCACCATCAAACAGGATTTTCGCCT
GCTGGGGCAAACCAGCGTGGACCGCTTGCTGCAACTCTCTCAGGGCCAGG
CGGTGAAGGGCAATCAGCTGTTGCCCGTCTCACTGGTGAAAAGAAAAACC
ACCCTGGCGCCCAATACGCAAACCGCCTCTCCCCGCGCGTTGGCCGATTC
ATTAATGCAGCTGGCACGACAGGTTTCCCGACTGGAAAGCGGGCAGTGAG
CGCAACGCAATTAATGTGAGTTAGCGCGAATTGATCTG(SEQ ID NO:
6)

In some embodiments, the vector comprises a nucleic acid sequence having at least 70% sequence identity to SEQ ID NO: 7. In some embodiments, the vector comprises a nucleic acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 7. Vector corresponding to SEQ ID NO: 7 is also referred to herein as pCL-BP2.

CGCGCGCGAAGGCGAAGCGGCATGCATTTACGTTGACACCATCGAATGGT
GCAAAACCTTTCGCGGTATGGCATGATAGCGCCCGGAAGAGAGTCAATTC
AGGGTGGTGAATGTGAAACCAGTAACGTTATACGATGTCGCAGAGTATGC
CGGTGTCTCTTATCAGACCGTTTCCCGCGTGGTGAACCAGGCCAGCCACG
TTTCTGCGAAAACGCGGGAAAAAGTGGAAGCGGCGATGGCGGAGCTGAAT
TACATTCCCAACCGCGTGGCACAACAACTGGCGGGCAAACAGTCGTTGCT
GATTGGCGTTGCCACCTCCAGTCTGGCCCTGCACGCGCCGTCGCAAATTG
TCGCGGCGATTAAATCTCGCGCCGATCAACTGGGTGCCAGCGTGGTGGTG
TCGATGGTAGAACGAAGCGGCGTCGAAGCCTGTAAAGCGGCGGTGCACAA
TCTTCTCGCGCAACGCGTCAGTGGGCTGATCATTAACTATCCGCTGGATG
ACCAGGATGCCATTGCTGTGGAAGCTGCCTGCACTAATGTTCCGGCGTTA
TTTCTTGATGTCTCTGACCAGACACCCATCAACAGTATTATTTTCTCCCA
TGAAGACGGTACGCGACTGGGCGTGGAGCATCTGGTCGCATTGGGTCACC
AGCAAATCGCGCTGTTAGCGGGCCCATTAAGTTCTGTCTCGGCGCGTCTG
CGTCTGGCTGGCTGGCATAAATATCTCACTCGCAATCAAATTCAGCCGAT
AGCGGAACGGGAAGGCGACTGGAGTGCCATGTCCGGTTTTCAACAAACCA
TGCAAATGCTGAATGAGGGCATCGTTCCCACTGCGATGCTGGTTGCCAAC
GATCAGATGGCGCTGGGCGCAATGCGCGCCATTACCGAGTCCGGGCTGCG
CGTTGGTGCGGATATCTCGGTAGTGGGATACGACGATACCGAAGACAGCT
CATGTTATATCCCGCCGTCAACCACCATCAAACAGGATTTTCGCCTGCTG
GGGCAAACCAGCGTGGACCGCTTGCTGCAACTCTCTCAGGGCCAGGCGGT
GAAGGGCAATCAGCTGTTGCCCGTCTCACTGGTGAAAAGAAAAACCACCC
TGGCGCCCAATACGCAAACCGCCTCTCCCCGCGCGTTGGCCGATTCATTA
ATGCAGCTGGCACGACAGGTTTCCCGACTGGAAAGCGGGCAGTGAGCGCA
ACGCAATTAATGTGAGTTAGCGCGAATTGATCTGGTTTGACAGCTTATCA
TCGACTGCACGGTGCACCAATGCTTCTGGCGTCAGGCAGCCATCGGAAGC
TGTGGTATGGCTGTGCAGGTCGTAAATCACTGCATAATTCGTGTCGCTCA
AGGCGCACTCCCGTTCTGGATAATGTTTTTTGCGCCGACATCATAACGGT
TCTGGCAAATATTCTGAAATGAGCTGTTGACAATTAATCATCCGGCTCGT
ATAATGTGTGGAATTGTGAGCGGATAACAATTTCACACAGGAAACAGCGC
CGCTGAGAAAAAGCGAAGCGGCACTGCTCTTTAACAATTTATCAGACAAT
CTGTGTGGGCACTCGACCGGAATTATCGATTAACTTTATTATTAAAAATT
AAAGAGGTATATATTAATGTATCGATTAAATAAGGAGGAATAAACCGCTT
TCTAGAACAAAAACTCATCTCAGAAGAGGATCTGAATAGCGCCGTCGACC
ATCATCATCATCATCATTGAGTTTAAACCAAATAAAACGAAAGGCTCAGT
CGAAAGACTGGGCCTTTCGTTTTATCTGTTGTTTGTCGGTGAACGCTCTC
CTGAGTAGGACAAATCCGCCGGGAGCGGATTTGAACGTTGCGAAGCAACG
GCCCGGAGGGTGGCGGGCAGGACGCCCGCCATAAACTGCCAGGCATCAAA
TTAAGCAGAAGGCCATCCTGACGGATGGCCTTTTTGCGTTTCTACAAACT
CTTTATGTGCTTAGTGCATCTAACGCTTGAGTTAAGCCGCGCCGCGAAGC
GGCGTCGGCTTGAACGAATTGTTAGACATTATTTGCCGACTACCTTGGTG
ATCTCGCCTTTCACGTAGTGGACAAATTCTTCCAACTGATCTGCGCGCGA
GGCCAAGCGATCTTCTTCTTGTCCAAGATAAGCCTGTCTAGCTTCAAGTA
TGACGGGCTGATACTGGGCCGGCAGGCGCTCCATTGCCCAGTCGGCAGCG
ACATCCTTCGGCGCGATTTTGCCGGTTACTGCGCTGTACCAAATGCGGGA
CAACGTAAGCACTACATTTCGCTCATCGCCAGCCCAGTCGGGCGGCGAGT
TCCATAGCGTTAAGGTTTCATTTAGCGCCTCAAATAGATCCTGTTCAGGA
ACCGGATCAAAGAGTTCCTCCGCCGCTGGACCTACCAAGGCAACGCTATG
TTCTCTTGCTTTTGTCAGCAAGATAGCCAGATCAATGTCGATCGTGGCTG
GCTCGAAGATACCTGCAAGAATGTCATTGCGCTGCCATTCTCCAAATTGC
AGTTCGCGCTTAGCTGGATAACGCCACGGAATGATGTCGTCGTGCACAAC
AATGGTGACTTCTACAGCGCGGAGAATCTCGCTCTCTCCAGGGGAAGCCG
AAGTTTCCAAAAGGTCGTTGATCAAAGCTCGCCGCGTTGTTTCATCAAGC
CTTACGGTCACCGTAACCAGCAAATCAATATCACTGTGTGGCTTCAGGCC
GCCATCCACTGCGGAGCCGTACAAATGTACGGCCAGCAACGTCGGTTCGA
GATGGCGCTCGATGACGCCAACTACCTCTGATAGTTGAGTCGATACTTCG
GCGATCACCGCTTCCCTCATGATGTTTAACTTTGTTTTAGGGCGACTGCC
CTGCTGCGTAACATCGTTGCTGCTCCATAACATCAAACATCGACCCACGG
CGTAACGCGCTTGCTGCTTGGATGCCCGAGGCATAGACTGTACCCCAAAA
AAACAGTCATAACAAGCCATGAAAACCGCCACTGCGCCGTTACCACCGCT
GCGTTCGGTCAAGGTTCTGGACCAGTTGCGTGAGCGCATACGCTACTTGC
ATTACAGCTTACGAACCGAACAGGCTTATGTCCACTGGGTTCGTGCCTTC
ATCCGTTTCCACGGTGTGCGTCACCCGGCAACCTTGGGCAGCAGCGAAGT
CGAGGCATTTCTGTCCTGGCTGGCGAACGAGCGCAAGGTTTCGGTCTCCA
CGCATCGTCAGGCATTGGCGGCCTTGCTGTTCTTCTACGGCAAGGTGCTG
TGCACGGATCTGCCCTGGCTTCAGGAGATCGGAAGACCTCGGCCGTCGCG
GCGCTTGCCGGTGGTGCTGACCCCGGATGAAGTGGTTCGCATCCTCGGTT
TTCTGGAAGGCGAGCATCGTTTGTTCGCCCAGCTTCTGTATGGAACGGGC
ATGCGGATCAGTGAGGGTTTGCAACTGCGGGTCAAGGATCTGGATTTCGA
TCACGGCACGATCATCGTGCGGGAGGGCAAGGGCTCCAAGGATCGGGCCT
TGATGTTACCCGAGAGCTTGGCACCCAGCCTGCGCGAGCAGGGGAATTAA
TTCCCACGGGTTTTGCTGCCCGCAAACGGGCTGTTCTGGTGTTGCTAGTT
TGTTATCAGAATCGCAGATCCGGCTTCAGCCGGTTTGCCGGCTGAAAGCG
CTATTTCTTCCAGAATTGCCATGATTTTTTCCCCACGGGAGGCGTCACTG
GCTCCCGTGTTGTCGGCAGCTTTGATTCGATAAGCAGCATCGCCTGTTTC
AGGCTGTCTATGTGTGACTGTTGAGCTGTAACAAGTTGTCTCAGGTGTTC
AATTTCATGTTCTAGTTGCTTTGTTTTACTGGTTTCACCTGTTCTATTAG
GTGTTACATGCTGTTCATCTGTTACATTGTCGATCTGTTCATGGTGAACA
GCTTTGAATGCACCAAAAACTCGTAAAAGCTCTGATGTATCTATCTTTTT
TACACCGTTTTCATCTGTGCATATGGACAGTTTTCCCTTTGATATGTAAC
GGTGAACAGTTGTTCTACTTTTGTTTGTTAGTCTTGATGCTTCACTGATA
GATACAAGAGCCATAAGAACCTCAGATCCTTCCGTATTTAGCCAGTATGT
TCTCTAGTGTGGTTCGTTGTTTTTGCGTGAGCCATGAGAACGAACCATTG
AGATCATACTTACTTTGCATGTCACTCAAAAATTTTGCCTCAAAACTGGT
GAGCTGAATTTTTGCAGTTAAAGCATCGTGTAGTGTTTTTCTTAGTCCGT
TATGTAGGTAGGAATCTGATGTAATGGTTGTTGGTATTTTGTCACCATTC
ATTTTTATCTGGTTGTTCTCAAGTTCGGTTACGAGATCCATTTGTCTATC
TAGTTCAACTTGGAAAATCAACGTATCAGTCGGGCGGCCTCGCTTATCAA
CCACCAATTTCATATTGCTGTAAGTGTTTAAATCTTTACTTATTGGTTTC
AAAACCCATTGGTTAAGCCTTTTAAACTCATGGTAGTTATTTTCAAGCAT
TAACATGAACTTAAATTCATCAAGGCTAATCTCTATATTTGCCTTGTGAG
TTTTCTTTTGTGTTAGTTCTTTTAATAACCACTCATAAATCCTCATAGAG
TATTTGTTTTCAAAAGACTTAACATGTTCCAGATTATATTTTATGAATTT
TTTTAACTGGAAAAGATAAGGCAATATCTCTTCACTAAAAACTAATTCTA
ATTTTTCGCTTGAGAACTTGGCATAGTTTGTCCACTGGAAAATCTCAAAG
CCTTTAACCAAAGGATTCCTGATTTCCACAGTTCTCGTCATCAGCTCTCT
GGTTGCTTTAGCTAATACACCATAAGCATTTTCCCTACTGATGTTCATCA
TCTGAGCGTATTGGTTATAAGTGAACGATACCGTCCGTTCTTTCCTTGTA
GGGTTTTCAATCGTGGGGTTGAGTAGTGCCACACAGCATAAAATTAGCTT
GGTTTCATGCTCCGTTAAGTCATAGCGACTAATCGCTAGTTCATTTGCTT
TGAAAACAACTAATTCAGACATACATCTCAATTGGTCTAGGTGATTTTAA
TCACTATACCAATTGAGATGGGCTAGTCAATGATAATTACTAGTCCTTTT
CCTTTGAGTTGTGGGTATCTGTAAATTCTGCTAGACCTTTGCTGGAAAAC
TTGTAAATTCTGCTAGACCCTCTGTAAATTCCGCTAGACCTTTGTGTGTT
TTTTTTGTTTATATTCAAGTGGTTATAATTTATAGAATAAAGAAAGAATA
AAAAAAGATAAAAAGAATAGATCCCAGCCCTGTGTATAACTCACTACTTT
AGTCAGTTCCGCAGTATTACAAAAGGATGTCGCAAACGCTGTTTGCTCCT
CTACAAAACAGACCTTAAAACCCTAAAGGCTTAAGTAGCACCCTCGCAAG
CTCGGGCAAATCGCTGAATATTCCTTTTGTCTCCGACCATCAGGCACCTG
AGTCGCTGTCTTTTTCGTGACATTCAGTTCGCTGCGCTCACGGCTCTGGC
AGTGAATGGGGGTAAATGGCACTACAGGCGCCTTTTATGGATTCATGCAA
GGAAACTACCCATAATACAAGAAAAGCCCGTCACGGGCTTCTCAGGGCGT
TTTATGGCGGGTCTGCTATGTGGTGCTATCTGACTTTTTGCTGTTCAGCA
GTTCCTGCCCTCTGATTTTCCAGTCTGACCACTTCGGATTATCCCGTGAC
AGGTCATTCAGACTGGCTAATGCACCCAGTAAGGCAGCGGTATCATCAAC
AGGCTTACCCGTCTTACTGT (SEQ ID NO: 7)

In some embodiments, the vector comprises a nucleic acid sequence having at least 70% sequence identity to SEQ ID NO: 8. The vector of SEQ ID NO: 8 corresponds to the vector of SEQ ID NO: 7 comprising the nucleic acid of SEQ ID NO: 4. In some embodiments, the vector comprises a nucleic acid sequence having at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 8. Vector corresponding to SEQ ID NO: 8 is also referred to herein as pAchro-L1.

CGCGCGCGAAGGCGAAGCGGCATGCATTTACGTTGACACCATCGAATGGT
GCAAAACCTTTCGCGGTATGGCATGATAGCGCCCGGAAGAGAGTCAATTC
AGGGTGGTGAATGTGAAACCAGTAACGTTATACGATGTCGCAGAGTATGC
CGGTGTCTCTTATCAGACCGTTTCCCGCGTGGTGAACCAGGCCAGCCACG
TTTCTGCGAAAACGCGGGAAAAAGTGGAAGCGGCGATGGCGGAGCTGAAT
TACATTCCCAACCGCGTGGCACAACAACTGGCGGGCAAACAGTCGTTGCT
GATTGGCGTTGCCACCTCCAGTCTGGCCCTGCACGCGCCGTCGCAAATTG
TCGCGGCGATTAAATCTCGCGCCGATCAACTGGGTGCCAGCGTGGTGGTG
TCGATGGTAGAACGAAGCGGCGTCGAAGCCTGTAAAGCGGCGGTGCACAA
TCTTCTCGCGCAACGCGTCAGTGGGCTGATCATTAACTATCCGCTGGATG
ACCAGGATGCCATTGCTGTGGAAGCTGCCTGCACTAATGTTCCGGCGTTA
TTTCTTGATGTCTCTGACCAGACACCCATCAACAGTATTATTTTCTCCCA
TGAAGACGGTACGCGACTGGGCGTGGAGCATCTGGTCGCATTGGGTCACC
AGCAAATCGCGCTGTTAGCGGGCCCATTAAGTTCTGTCTCGGCGCGTCTG
CGTCTGGCTGGCTGGCATAAATATCTCACTCGCAATCAAATTCAGCCGAT
AGCGGAACGGGAAGGCGACTGGAGTGCCATGTCCGGTTTTCAACAAACCA
TGCAAATGCTGAATGAGGGCATCGTTCCCACTGCGATGCTGGTTGCCAAC
GATCAGATGGCGCTGGGCGCAATGCGCGCCATTACCGAGTCCGGGCTGCG
CGTTGGTGCGGATATCTCGGTAGTGGGATACGACGATACCGAAGACAGCT
CATGTTATATCCCGCCGTCAACCACCATCAAACAGGATTTTCGCCTGCTG
GGGCAAACCAGCGTGGACCGCTTGCTGCAACTCTCTCAGGGCCAGGCGGT
GAAGGGCAATCAGCTGTTGCCCGTCTCACTGGTGAAAAGAAAAACCACCC
TGGCGCCCAATACGCAAACCGCCTCTCCCCGCGCGTTGGCCGATTCATTA
ATGCAGCTGGCACGACAGGTTTCCCGACTGGAAAGCGGGCAGTGAGCGCA
ACGCAATTAATGTGAGTTAGCGCGAATTGATCTGGTTTGACAGCTTATCA
TCGACTGCACGGTGCACCAATGCTTCTGGCGTCAGGCAGCCATCGGAAGC
TGTGGTATGGCTGTGCAGGTCGTAAATCACTGCATAATTCGTGTCGCTCA
AGGCGCACTCCCGTTCTGGATAATGTTTTTTGCGCCGACATCATAACGGT
TCTGGCAAATATTCTGAAATGAGCTGTTGACAATTAATCATCCGGCTCGT
ATAATGTGTGGAATTGTGAGCGGATAACAATTTCACACAGGAAACAGCGC
CGCTGAGAAAAAGCGAAGCGGCACTGCTCTTTAACAATTTATCAGACAAT
CTGTGTGGGCACTCGACCGGAATTATCGATTAACTTTATTATTAAAAATT
AAAGAGGTATATATTAATGTATCGATTAAATAAGGAGGAATAAACCATGT
CGCAAACTCCTCGCAAACTGCGTTCGCAGAAGTGGTTTGACGATCCGGCA
CATGCAGATATGACGGCCATCTATGTCGAACGTTACTTGAACTACGGGTT
GACCCGTCAAGAGCTGCAAAGTGGCCGCCCGATCATTGGAATTGCCCAAA
CTGGGTCCGATTTAGCTCCTTGTAACCGCCATCATCTTGCACTTGCTGAG
CGTATCAAGGCCGGAATTCGTGACGCCGGTGGCATCCCGATGGAATTTCC
TGTGCATCCCTTGGCGGAACAGGGCCGTCGTCCAACTGCGGCCTTAGACC
GCAACTTAGCATATCTTGGGCTGGTCGAAATCCTGCACGGGTATCCCTTG
GACGGAGTTGTGCTGACGACAGGTTGCGACAAGACCACCCCTGCTTGTCT
GATGGCAGCTGCAACAGTGGACATCCCGGCGATTGTGCTGAGCGGCGGAC
CCATGCTTGACGGGTGGCACGACGGCCAGCGCGTGGGCAGTGGAACAGTG
ATCTGGCACGCGCGTAATTTAATGGCAGCGGGTAAATTAGATTACGAAGG
TTTTATGACACTGGCCACTGCTAGCAGTCCCTCCGTCGGTCATTGCAACA
CAATGGGGACGGCACTTAGCATGAACTCGCTGGCAGAAGCTCTGGGAATG
TCCTTACCGACATGCGCATCAATCCCGGCCCCCTATCGCGAACGTGGGCA
AATGGCATACGCGACTGGTTTGCGCATCTGCGACATGGTACGTGAGGATC
TTCGTCCCTCGCACATCCTTACTCGTCAGGCCTTTGAAAACGCGATTGTC
GTTGCCTCAGCTTTGGGCGCGAGTAGTAACTGCCCACCTCATTTGATTGC
GATGGCCCGTCACGCCGGAATCGATTTATCGCTTGATGATTGGCAACGCC
TTGGAGAAGATGTTCCGTTGCTTGTCAATTGCGTTCCCGCGGGTGAGCAT
CTTGGGGAGGGATTCCACCGTGCCGGGGGCGTACCTGCCGTTCTTCACGA
ATTGGCAGCGGCTGGGCGCCTTCACACCGACTGCGGAACCGTTTCCGGGA
AAACGATCGGCGAGATCGCAGCAACAGCCAAGACTAATAACGCAGATGTA
ATCCGTTCTTGTGATGCTCCCCTTCGTCACCGCGCCGGGTTCATCGTATT
ATCAGGGAACTTTTTTGACTCCGCCATCATCAAAATGTCGGTCGTAGGCG
AGGCATTCCGCCGCGCATACTTGTCGGAACCGGGTTCAGAAAATGCGTTC
GAAGCTCGTGCGATTGTGTTTGAGGGCCCAGAAGACTACCATGCGCGCAT
TGAAGATCCTTCTCTTAATATTGACGAACATTGTATCCTGGTAATCCGCG
GAGCGGGGACGGTGGGTTACCCAGGTTCGGCGGAAGTAGTCAATATGGCT
CCTCCCAGTCACCTTTTAAAACGCGGTATTGACTCATTACCGTGTTTAGG
GGATGGTCGCCAGAGCGGAACGAGTGCATCTCCCAGCATCTTGAATATGT
CTCCAGAGGCCGCGGTCGGGGGAGGCTTGGCGCTTCTGCGCACCGGTGAC
CGCATTCGTGTTGACCTGAACCAGCGCTCAGTTACGGCGCTTGTCGATGA
GACTGAAATGGAGCGTCGTAAATTGGAACCACCTTATCAGGCCCCAGAAT
CGCAGACCCCGTGGCAAGAGTTGTATCGCCAGTTAGTTGGGCAATTGTCC
ACGGGAGGATGTCTTGAGCCCGCGACATTGTACCTTAAGGTCGTAGAGAC
ACGTGGTGATCCGCGTCACTCTCACTAAGCTTTCTAGAACAAAAACTCAT
CTCAGAAGAGGATCTGAATAGCGCCGTCGACCATCATCATCATCATCATT
GAGTTTAAACCAAATAAAACGAAAGGCTCAGTCGAAAGACTGGGCCTTTC
GTTTTATCTGTTGTTTGTCGGTGAACGCTCTCCTGAGTAGGACAAATCCG
CCGGGAGCGGATTTGAACGTTGCGAAGCAACGGCCCGGAGGGTGGCGGGC
AGGACGCCCGCCATAAACTGCCAGGCATCAAATTAAGCAGAAGGCCATCC
TGACGGATGGCCTTTTTGCGTTTCTACAAACTCTTTATGTGCTTAGTGCA
TCTAACGCTTGAGTTAAGCCGCGCCGCGAAGCGGCGTCGGCTTGAACGAA
TTGTTAGACATTATTTGCCGACTACCTTGGTGATCTCGCCTTTCACGTAG
TGGACAAATTCTTCCAACTGATCTGCGCGCGAGGCCAAGCGATCTTCTTC
TTGTCCAAGATAAGCCTGTCTAGCTTCAAGTATGACGGGCTGATACTGGG
CCGGCAGGCGCTCCATTGCCCAGTCGGCAGCGACATCCTTCGGCGCGATT
TTGCCGGTTACTGCGCTGTACCAAATGCGGGACAACGTAAGCACTACATT
TCGCTCATCGCCAGCCCAGTCGGGCGGCGAGTTCCATAGCGTTAAGGTTT
CATTTAGCGCCTCAAATAGATCCTGTTCAGGAACCGGATCAAAGAGTTCC
TCCGCCGCTGGACCTACCAAGGCAACGCTATGTTCTCTTGCTTTTGTCAG
CAAGATAGCCAGATCAATGTCGATCGTGGCTGGCTCGAAGATACCTGCAA
GAATGTCATTGCGCTGCCATTCTCCAAATTGCAGTTCGCGCTTAGCTGGA
TAACGCCACGGAATGATGTCGTCGTGCACAACAATGGTGACTTCTACAGC
GCGGAGAATCTCGCTCTCTCCAGGGGAAGCCGAAGTTTCCAAAAGGTCGT
TGATCAAAGCTCGCCGCGTTGTTTCATCAAGCCTTACGGTCACCGTAACC
AGCAAATCAATATCACTGTGTGGCTTCAGGCCGCCATCCACTGCGGAGCC
GTACAAATGTACGGCCAGCAACGTCGGTTCGAGATGGCGCTCGATGACGC
CAACTACCTCTGATAGTTGAGTCGATACTTCGGCGATCACCGCTTCCCTC
ATGATGTTTAACTTTGTTTTAGGGCGACTGCCCTGCTGCGTAACATCGTT
GCTGCTCCATAACATCAAACATCGACCCACGGCGTAACGCGCTTGCTGCT
TGGATGCCCGAGGCATAGACTGTACCCCAAAAAAACAGTCATAACAAGCC
ATGAAAACCGCCACTGCGCCGTTACCACCGCTGCGTTCGGTCAAGGTTCT
GGACCAGTTGCGTGAGCGCATACGCTACTTGCATTACAGCTTACGAACCG
AACAGGCTTATGTCCACTGGGTTCGTGCCTTCATCCGTTTCCACGGTGTG
CGTCACCCGGCAACCTTGGGCAGCAGCGAAGTCGAGGCATTTCTGTCCTG
GCTGGCGAACGAGCGCAAGGTTTCGGTCTCCACGCATCGTCAGGCATTGG
CGGCCTTGCTGTTCTTCTACGGCAAGGTGCTGTGCACGGATCTGCCCTGG
CTTCAGGAGATCGGAAGACCTCGGCCGTCGCGGCGCTTGCCGGTGGTGCT
GACCCCGGATGAAGTGGTTCGCATCCTCGGTTTTCTGGAAGGCGAGCATC
GTTTGTTCGCCCAGCTTCTGTATGGAACGGGCATGCGGATCAGTGAGGGT
TTGCAACTGCGGGTCAAGGATCTGGATTTCGATCACGGCACGATCATCGT
GCGGGAGGGCAAGGGCTCCAAGGATCGGGCCTTGATGTTACCCGAGAGCT
TGGCACCCAGCCTGCGCGAGCAGGGGAATTAATTCCCACGGGTTTTGCTG
CCCGCAAACGGGCTGTTCTGGTGTTGCTAGTTTGTTATCAGAATCGCAGA
TCCGGCTTCAGCCGGTTTGCCGGCTGAAAGCGCTATTTCTTCCAGAATTG
CCATGATTTTTTCCCCACGGGAGGCGTCACTGGCTCCCGTGTTGTCGGCA
GCTTTGATTCGATAAGCAGCATCGCCTGTTTCAGGCTGTCTATGTGTGAC
TGTTGAGCTGTAACAAGTTGTCTCAGGTGTTCAATTTCATGTTCTAGTTG
CTTTGTTTTACTGGTTTCACCTGTTCTATTAGGTGTTACATGCTGTTCAT
CTGTTACATTGTCGATCTGTTCATGGTGAACAGCTTTGAATGCACCAAAA
ACTCGTAAAAGCTCTGATGTATCTATCTTTTTTACACCGTTTTCATCTGT
GCATATGGACAGTTTTCCCTTTGATATGTAACGGTGAACAGTTGTTCTAC
TTTTGTTTGTTAGTCTTGATGCTTCACTGATAGATACAAGAGCCATAAGA
ACCTCAGATCCTTCCGTATTTAGCCAGTATGTTCTCTAGTGTGGTTCGTT
GTTTTTGCGTGAGCCATGAGAACGAACCATTGAGATCATACTTACTTTGC
ATGTCACTCAAAAATTTTGCCTCAAAACTGGTGAGCTGAATTTTTGCAGT
TAAAGCATCGTGTAGTGTTTTTCTTAGTCCGTTATGTAGGTAGGAATCTG
ATGTAATGGTTGTTGGTATTTTGTCACCATTCATTTTTATCTGGTTGTTC
TCAAGTTCGGTTACGAGATCCATTTGTCTATCTAGTTCAACTTGGAAAAT
CAACGTATCAGTCGGGCGGCCTCGCTTATCAACCACCAATTTCATATTGC
TGTAAGTGTTTAAATCTTTACTTATTGGTTTCAAAACCCATTGGTTAAGC
CTTTTAAACTCATGGTAGTTATTTTCAAGCATTAACATGAACTTAAATTC
ATCAAGGCTAATCTCTATATTTGCCTTGTGAGTTTTCTTTTGTGTTAGTT
CTTTTAATAACCACTCATAAATCCTCATAGAGTATTTGTTTTCAAAAGAC
TTAACATGTTCCAGATTATATTTTATGAATTTTTTTAACTGGAAAAGATA
AGGCAATATCTCTTCACTAAAAACTAATTCTAATTTTTCGCTTGAGAACT
TGGCATAGTTTGTCCACTGGAAAATCTCAAAGCCTTTAACCAAAGGATTC
CTGATTTCCACAGTTCTCGTCATCAGCTCTCTGGTTGCTTTAGCTAATAC
ACCATAAGCATTTTCCCTACTGATGTTCATCATCTGAGCGTATTGGTTAT
AAGTGAACGATACCGTCCGTTCTTTCCTTGTAGGGTTTTCAATCGTGGGG
TTGAGTAGTGCCACACAGCATAAAATTAGCTTGGTTTCATGCTCCGTTAA
GTCATAGCGACTAATCGCTAGTTCATTTGCTTTGAAAACAACTAATTCAG
ACATACATCTCAATTGGTCTAGGTGATTTTAATCACTATACCAATTGAGA
TGGGCTAGTCAATGATAATTACTAGTCCTTTTCCTTTGAGTTGTGGGTAT
CTGTAAATTCTGCTAGACCTTTGCTGGAAAACTTGTAAATTCTGCTAGAC
CCTCTGTAAATTCCGCTAGACCTTTGTGTGTTTTTTTTGTTTATATTCAA
GTGGTTATAATTTATAGAATAAAGAAAGAATAAAAAAAGATAAAAAGAAT
AGATCCCAGCCCTGTGTATAACTCACTACTTTAGTCAGTTCCGCAGTATT
ACAAAAGGATGTCGCAAACGCTGTTTGCTCCTCTACAAAACAGACCTTAA
AACCCTAAAGGCTTAAGTAGCACCCTCGCAAGCTCGGGCAAATCGCTGAA
TATTCCTTTTGTCTCCGACCATCAGGCACCTGAGTCGCTGTCTTTTTCGT
GACATTCAGTTCGCTGCGCTCACGGCTCTGGCAGTGAATGGGGGTAAATG
GCACTACAGGCGCCTTTTATGGATTCATGCAAGGAAACTACCCATAATAC
AAGAAAAGCCCGTCACGGGCTTCTCAGGGCGTTTTATGGCGGGTCTGCTA
TGTGGTGCTATCTGACTTTTTGCTGTTCAGCAGTTCCTGCCCTCTGATTT
TCCAGTCTGACCACTTCGGATTATCCCGTGACAGGTCATTCAGACTGGCT
AATGCACCCAGTAAGGCAGCGGTATCATCAACAGGCTTACCCGTCTTACT
GT (SEQ ID NO: 8)

### Recombinant Cells

KDG is a common intermediate in the normal metabolism of some microorganisms, and KDG production is closely associated with cell growth. Normal metabolism and normal cell growth lead to consumption of substrates typically involved in the production of KDG. Thus, normal metabolism and normal cell growth restrict the amount of KDG that may accumulate within (and thus be recovered from) a cell. The inventors have made the surprising discovery that competing functions of cell growth and metabolism may be uncoupled from KDG production, by introducing targeted genetic modifications to the cell. By uncoupling KDG production from cell growth and metabolism, the inventors have achieved an unexpected and advantageous improvement in KDG production and recovery.

To achieve the advantageous improvement in KDG production and recovery, recombinant cells of the disclosure may comprise one or more genetic modifications resulting in one or more of the following phenotypes:

### Decrease or Elimination of Glucose Assimilation

Glucose is the preferred carbon source for many microorganisms, including E. coli, and is assimilated by various endogenous pathways. As used herein, the term “glucose assimilation” refers to any cellular process that metabolizes glucose. This includes, but is not limited to, uptake of glucose into the cytoplasm, glucose phosphorylation, glycogen biosynthesis, and glucose isomerization. According to the disclosure, decreasing or eliminating glucose assimilation reduces or prevents glucose metabolism by the cell’s endogenous metabolic pathways. Glucose that is not assimilated may be converted to gluconate by a glucose dehydrogenase and then converted to KDG by gluconate dehydratase. Decreasing or eliminating glucose assimilation may comprise decreasing expression, deletion or inactivation of at least one endogenous gene involved in cellular process(es) that metabolize glucose. In many microorganisms, the phosphoenolpyruvate (PEP) dependent carbohydrate-phosphotransferase system is the preferred transport system for glucose uptake into the cytoplasm. Thus, decreasing or eliminating glucose assimilation may include decreasing expression, deletion or inactivation of at least one gene involved in the phosphoenolpyruvate (PEP) dependent carbohydrate-phosphotransferase system. In certain embodiments, the at least one gene involved in the PEP dependent carbohydrate-phosphotransferase system is selected from ptsl, ptsH and/or crr.

### Decrease or Elimination of Gluconate-6-Phosphate Production From Gluconate

Gluconate may be transported into cells by endogenous transporters. Once intracellular, gluconate is phosphorylated to produce gluconate-6-phosphate and metabolized by endogenous metabolic pathways. According to the disclosure, decreasing or eliminating gluconate-6-phosphate production reduces or prevents direct metabolism of gluconate by the cell’s endogenous metabolic pathways, thereby increasing the availability of gluconate as a substrate for gluconate dehydratase. Decreasing or eliminating gluconate-6-phosphate production may be achieved by decreasing expression, deletion or inactivation of at least one gene encoding a gluconate kinase. In certain embodiments, the at least one gene encoding a gluconate kinase is selected from gntK and/or idnK.

### Decrease or Elimination Ofglucose-6-Phosphate Production From Glucose

Glucose may be transported into the cell by various non-specific transporters or may be produced by the breakdown of more complex sugars, e.g. cellobiose. Glucose accumulated by these processes can be phosphorylated to produce glucose-6-phosphate and directed to the central metabolism. According to the disclosure, decreasing or eliminating glucose-6-phosphate production reduces or prevents glucose metabolism by the cell’s endogenous metabolic pathways. Decreasing or eliminating glucose-6-phosphate production may be achieved by decreasing expression, deletion or inactivation of at least one gene encoding a glucokinase. In certain embodiments, the at least one gene encoding a glucokinase is glk.

### Decrease or Elimination of KDG Phosphorylation

KDG may be phosphorylated to KDPG and degraded by endogenous metabolic pathways. According to the disclosure, decreasing or eliminating KDG phosphorylation reduces or prevents degradation of KDG as part of endogenous cellular metabolism. Decreasing or eliminating KDG phosphorylation may be achieved by decreasing expression, deletion or inactivation of at least one gene encoding a 2-dehydro-3-deoxygluconokinase. In certain embodiments, the at least one gene encoding a 2-dehydro-3-deoxygluconokinase is kdgK.

### Decrease or Elimination of KDG Degradation by Non-Phosphorylative Cellular Reactions That Consume KDG

KDG may be degraded by alternative endogenous cellular pathways which do not involve conversion to KDPG, e.g. KDG may be converted to glyceraldehyde and pyruvate via the non-phosphorylative Entner-Doudoroff (ED) pathway. According to the disclosure, decreasing or eliminating KDG degradation by non-phosphorylative cellular reactions that consume KDG reduces or prevents loss of KDG to cellular metabolism. Decreasing or eliminating KDG degradation by non-phosphorylative cellular reactions that consume KDG may be achieved by decreasing expression, deletion or inactivation of at least one gene encoding a KDG aldolase. In certain embodiments, the at least one gene encoding a KDG aldolase is selected from yjhH and/or yagE.

### Increased Export of KDG From the Recombinant Cell

KDG may be exported by the recombinant cell into the supernatant allowing more efficient recovery and purification. Without wishing to be bound by theory, the inventors believe that, in E. coli, KDG may be exported by various endogenous efflux mechanism(s) e.g. AcrAB-TolC, CusCFBA, AcrEF, EmrD, EmrE, EmrKY, HsrA, MdtAB, MdtEF, MdtBC, MdtD, MdtH, MdtJI, MdtK, MdtN, MdtO, MdtP. According to the disclosure, increased export of KDG may be achieved by increasing expression of at least one gene involved in a KDG efflux mechanism. In certain embodiments, the at least one gene involved in KDG efflux is selected from acrA, acrB, tolC, cusC, cusF, cusB, cusA, acrE, acrF, emrD, emrE, emrK, emrY, hsrA, mdtA, mdtB, mdtE, mdtF, mdtC, mdtD, mdtH, mdtJ, mdtl, mdtK, mdtN, mdtO and mdtP.

### Decreased Uptake of KDG Into the Recombinant Cell

To increase the concentration of extracellular KDG, it may be beneficial to decrease or eliminate (re)uptake of KDG into the recombinant cell. According to the disclosure, decreasing or eliminating KDG uptake may be achieved by decreasing expression, deletion or inactivation of at least one gene encoding a KDG permease. In some embodiments, the at least one gene encoding a KDG permease is kdgT.

### Increased Uptake of Gluconate Into the Recombinant Cell

Increased KDG production may be achieved by increasing uptake of gluconate into a cell. According to the disclosure, increased uptake of gluconate into a cell may be achieved by increasing the expression of at least one gene encoding a gluconate transporter. In some embodiments, the at least one gene encoding a gluconate transporter is selected from gntP, gntU, gntT, and/or idnT. In certain embodiments, the at least one gene encoding a gluconate transporter is selected from gntP and gntT.

In certain embodiments, the recombinant cell comprises: (a) one or more genetic modifications resulting in decrease or elimination of glucose assimilation; and (b) one or more genetic modifications resulting in decrease or elimination of gluconate-6-phosphate production from gluconate. Recombinant cells comprising both of these phenotypes are unable to direct glucose to central metabolism either directly, or via conversion of glucose to gluconate and then to gluconate-6-phosphate. Thus, in these cells, glucose has been uncoupled from cellular metabolism and growth. Glucose is converted to gluconate by glucose dehydrogenase.

In certain embodiments, recombinant cells comprise: (a) one or more genetic modifications resulting in decrease or elimination of glucose assimilation; and (b) one or more genetic modifications resulting in decrease or elimination of gluconate-6-phosphate production from gluconate; and further comprise a gene encoding a gluconate dehydratase. The increased content of glucose and gluconate in these modified recombinant cells leads to an advantageous increase in the amount of KDG produced by gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of KDG phosphorylation. Recombinant cells comprising all three of these phenotypes are unable to direct glucose to central metabolism either directly, or via conversion of glucose to gluconate and then to gluconate-6-phosphate. In these cells, glucose is converted to gluconate by glucose dehydrogenase. These recombinant cells are unable to direct KDG to central metabolism via the traditional ED pathway which requires KDG phosphorylation. In these cells, KDG produced by endogenous metabolic pathways accumulates in the cell.

In certain embodiments, recombinant cells comprise: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of KDG phosphorylation; and further comprising a gene encoding a gluconate dehydratase. The increased content of glucose and gluconate in these modified recombinant cells leads to an advantageous increase in the amount of KDG produced by gluconate dehydratase. In addition, the prevention of KDG phosphorylation leads to an advantageous increase in the accumulation of KDG in cells.

In certain embodiments, the recombinant cell further comprises one or more genetic modifications resulting in decrease or elimination of KDG degradation by non-phosphorylative cellular reactions that consume KDG. Recombinant cells comprising genetic modifications resulting in this phenotype exhibit increased accumulation (and recovery of) KDG because KDG cannot be directed to central metabolism by non-phosphorylative pathways, e.g. the non-phosphorylative ED pathway, and/or cannot be consumed by other KDG-consuming reactions.

In certain embodiments, the recombinant cell further comprises one or more genetic modifications resulting in decrease or elimination of glucose-6-phosphate production from glucose. Recombinant cells further comprising genetic modifications resulting in this phenotype exhibit more efficient uncoupling of cell growth from KDG production in the presence of glucose because these cells are unable to direct to central metabolism glucose which has been assimilated by nonspecific transporters or produced by the intracellular degradation of more complex sugars.

Various microorganisms produce KDG as an intermediate during the endogenous metabolism of several sugars/ sugar acids (e.g. galacturonate, glucuronate) and polysaccharides (e.g. pectin and alginate). Wherein KDG is produced in a cell solely by its endogenous metabolism, the recombinant cell typically comprises one or more genetic modifications resulting in the decrease or elimination of KDG phosphorylation and/or the decrease or elimination of KDG degradation by non-phosphorylative cellular reactions that consume KDG. Recombinant cells comprising genetic modifications resulting in these phenotypes exhibit uncoupled cell growth from KDG production because KDG produced by the endogenous metabolic pathways is prevented from entering central metabolism. Recombinant cells comprising these mutations typically require an alternative substrate for cell growth.

In certain embodiments, the recombinant cell comprises: (a) one or more genetic modifications resulting in decrease or elimination of glucose assimilation; and (b) one or more genetic modifications resulting in decrease or elimination of gluconate-6-phosphate production from gluconate.

In certain embodiments, the recombinant cell comprises: (a) one or more genetic modifications resulting in decrease or elimination of glucose assimilation; and (b) one or more genetic modifications resulting in decrease or elimination of gluconate-6-phosphate production from gluconate and further comprises a gene encoding a gluconate dehydratase.

In certain embodiments, the recombinant cell comprises one or more genetic modifications resulting in decrease or elimination of KDG phosphorylation.

In certain embodiments, the recombinant cell comprises one or more genetic modifications resulting in decrease or elimination of KDG phosphorylation and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of KDG phosphorylation.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate: and (c) one or more genetic modifications resulting in decrease or elimination of KDG phosphorylation; and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of KDG degradation by non-phosphorylative cellular reactions that consume KDG.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of KDG degradation by non-phosphorylative cellular reactions that consume KDG; and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; (c) one or more genetic modifications resulting in decrease or elimination of KDG phosphorylation; and (d) one or more genetic modifications resulting in decrease or elimination of KDG degradation by non-phosphorylative cellular reactions that consume KDG.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; (c) one or more genetic modifications resulting in decrease or elimination of KDG phosphorylation; and (d) one or more genetic modifications resulting in decrease or elimination of KDG degradation by non-phosphorylative cellular reactions that consume KDG; and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of glucose-6-phosphate production from glucose.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; and (c) one or more genetic modifications resulting in decrease or elimination of glucose-6-phosphate production from glucose, and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; (c) one or more genetic modifications resulting in decrease or elimination of KDG phosphorylation; and (d) one or more genetic modifications resulting in decrease or elimination of glucose-6-phosphate production from glucose.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; (c) one or more genetic modifications resulting in decrease or elimination of KDG phosphorylation; and (d) one or more genetic modifications resulting in decrease or elimination of glucose-6-phosphate production from glucose; and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; (c) one or more genetic modifications resulting in decrease or elimination of KDG phosphorylation; (d) one or more genetic modifications resulting in decrease or elimination of KDG degradation by non-phosphorylative cellular reactions that consume KDG; and (e) one or more genetic modifications resulting in decrease or elimination of glucose-6-phosphate production from glucose; and further comprises a gene encoding a gluconate dehydratase.

In certain embodiments, the recombinant cell comprises: (a) one or more genetic modifications resulting in decrease or elimination of glucose assimilation; (b) one or more genetic modifications resulting in decrease or elimination of gluconate-6-phosphate production from gluconate; (c) one or more genetic modifications resulting in decrease or elimination of KDG phosphorylation; (d) one or more genetic modifications resulting in decrease or elimination of KDG degradation by non-phosphorylative cellular reactions that consume KDG; and (e) one or more genetic modifications resulting in decrease or elimination of glucose-6-phosphate production from glucose; and further comprises a gene encoding a gluconate dehydratase, and further comprises a gene encoding a gluconate dehydratase.

The foregoing phenotypes may be introduced singly or in combinations of two, three, four, five, six, seven, eight or more modifications. One or more of the foregoing phenotypes may be obtained by increasing or decreasing expression of an endogenous protein (e.g., by at least a factor of about 1.1, about 1.2, about 1.5, about 2, about 5, about 10 or about 20) or a result of introducing expression of a heterologous polypeptide. The term “decreasing” or “reducing” gene expression encompasses eliminating or inactivating expression. Decreasing (or reducing) the expression of an endogenous protein may be accomplished by inactivating one or more (or all) endogenous copies of a gene in a cell. A gene may be inactivated by deletion of at least part of the gene or by disruption of the gene e.g. by deleting some or all of a gene coding sequence or regulatory sequence whose deletion results in a reduction of gene expression in the cell. Decreasing (or reducing) the expression of an endogenous protein may be accomplished by other methods known in the art, e.g. post-transcriptional gene silencing, such as RNA interference. Increasing the expression of a gene may be accomplished methods known in the art, e.g. by cloning the gene of interest into an expression vector under the control of a strong promoter.

In certain embodiments, the recombinant cell of the disclosure comprises a gene encoding a polypeptide, wherein said polypeptide comprises an amino acid sequence having at least 70% sequence identity to SEQ ID NO: 1. In such embodiments, the gene is typically a heterologous gene. In some embodiments, the recombinant cell comprises a gene encoding a polypeptide comprising an amino acid sequence having at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 1.

In certain embodiments, the recombinant cell of the disclosure comprises a gene encoding a heterologous gluconate dehydratase comprising a nucleic acid sequence having at least 70% sequence identity to SEQ ID NO: 2 or 3. In certain embodiments, the recombinant cell of the disclosure comprises a gene encoding a heterologous gluconate dehydratase comprising a nucleic acid sequence having at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 2 or 3.

In certain embodiments, the recombinant cell of the disclosure comprises a gene encoding a heterologous gluconate dehydratase comprising a nucleic acid sequence having at least 70% sequence identity to SEQ ID NO: 4. In certain embodiments, the recombinant cell of the disclosure comprises a gene encoding a heterologous gluconate dehydratase comprising a nucleic acid sequence having at least 75%, at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO: 4.

The gene encoding a heterologous gluconate dehydratase may be extrachromosomal, on a vector (typically a plasmid), which can be a low copy number vector, an intermediate copy number vector, or a high copy number vector. In some embodiments, the recombinant cell is transformed with a vector of the disclosure. In some embodiments, the recombinant cell is transiently transformed. The nucleic acid may be maintained episomally and thus comprise a sequence for autonomous replication, such as an autosomal replication sequence. Alternatively, the recombinant cell may be stably transformed wherein the nucleic acid is integrated in one or more copies into the genome of the cell. Integration into the cell’s genome may occur at random by non-homologous recombination but preferably, the nucleic acid construct may be integrated into the cell’s genome by homologous recombination, as is well known in the art.

In certain embodiments, the recombinant cell comprises a vector, wherein said vector comprises a gene encoding a polypeptide comprising an amino acid sequence having at least 70% sequence identity to SEQ ID NO: 1. In certain embodiments, the vector comprises a gene encoding a heterologous gluconate dehydratase, wherein the gene comprises a nucleic acid sequence having at least 70% sequence identity to SEQ ID NO: 2 or 3. In certain embodiments, the vector comprises a gene encoding a heterologous gluconate dehydratase, wherein the gene comprises a nucleic acid sequence having at least 70% sequence identity to SEQ ID NO: 4.

As described above, a vector according to the disclosure may comprise: an origin of replication; a promoter sequence operably linked to a gene encoding a heterologous gluconate dehydratase; and/or a reporter gene.

In certain embodiments, the vector comprises a nucleic acid sequence having at least 70% sequence identity to SEQ ID NO: 5, 6, 7, or 8.

In some embodiments, the recombinant cell is a prokaryotic cell. In some embodiments, the prokaryotic cell is selected from Acinetobacter, Agrobacterium, Escherichia, Cupriavidus, Clostridium, Rhodobacter, Marinobacter, Bacillus, Klebsiella, Tatumella, Pseudomonas, Ralstonia, Rhodococcus, Methylobacterium, Methylophilus, Methylococcus, Methylomicrobium, Methylomonas, Pantoea, Streptomyces, Zymomonas, Parachlorella, Synechococcus, Synechocystis and Thermocynechococcus. In some embodiments, the prokaryotic cell is E. coli. Suitable cells of the bacterial genera include, but are not limited to, cells of Lactobacillus, Pseudomonas, and Streptomyces. Suitable cells of bacterial species include, but are not limited to, cells of Bacillus subtilis, Bacillus licheniformis, Lactobacillus brevis, Pseudomonas aeruginosa, Zymomonas mobilis and Streptomyces lividans.

In some embodiments, the recombinant cell is a eukaryotic cell. In some embodiments, the eukaryotic cell is selected from the group consisting of a yeast cell, a fungal cell, and an algal cell. In some embodiments, the yeast cell is selected from the group consisting of Saccharomyces, Kluyveromyces, Candida, Pichia, Schizosaccharomyces, Schefferomyces, Rhodosporidium, Hansenula, Klockera, Schwanniomyces, Issatchenkia, Yarrowia and Rhodotorula. In some embodiments, the yeast cell is selected from the group consisting of S. cerevisiae, C. lipolytica, R. glutinis, S. bulderi, S. barnetti, S. exiguus, S. uvarum, S. diastaticus, K. lactis, K. marxianus, K. fragile, P. kudriavzevii, S. stipitis and I. orientalis. Suitable cells of yeast include, but are not limited to C. albicans, S. pombe, H. polymorpha, P. pastoris, P. canadensis, or P. rhodozyma.

In some embodiments, the fungal cell is a filamentous fungal cell. In some embodiments, the filamentous fungal cell is selected from the group consisting of Aspergillus, Penicillium, Rhizopus, Chrysosporium, Myceliophthora, Trichoderma, Humicola, Acremonium and Fusarium. In some embodiments, the filamentous fungal cell is selected from the group consisting of A. niger, A. oryzae, T. reesei, P. chrysogenum, M. thermophila, and R. oryzae. Suitable cells of filamentous fungal genera include, but are not limited to, cells of Aureobasidium, Bjerkandera, Ceriporiopsis, Coprinus, Coriolus, Corynascus, Chaetomium, Cryptococcus, Filobasidium, Gibberella, Hypocrea, Magnaporthe, Mucor, Neocallimastix, Neurospora, Paecilomyces, Phanerochaete, Phlebia, Piromyces, Pleurotus, Scytaldium, Schizophyllum, Sporotrichum, Talaromyces, Thermoascus, Thielavia, tolypocladium and Trametes. In certain aspects, the recombinant cell is a Trichoderma sp., Penicillium sp., Humicola sp. (e.g., Humicola insolens); Aspergillus sp., Chrysosporium sp., Fusarium sp., or Hypocrea sp.. Suitable cells can also include cells of various anamorph and teleomorph forms of these filamentous fungal genera.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

In some embodiments, the algal cell is selected from the group consisting of Botryococcus, Nannochloropsis, Chlorella, Chlamydomonas, Dunaliella, Chaetoceros, Porphyridium, Scenedesmus and Pseudochlorococcum. In some embodiments, the algal cell is selected from the group consisting of B. braunii and N. gaditana.

Recombinant cells may be cultured in conventional nutrient media modified as appropriate for activating promoters (if an inducible promoter is present), selecting transformants, or amplifying the nucleic acid sequence encoding the gluconate dehydratase polypeptide. Culture conditions, such as temperature, pH and the like, are those previously used with the recombinant cell selected for expression, and will be apparent to those skilled in the art. Many references are available for the culture and production of many cells, including cells of bacterial and fungal origin. Cell culture media in general are set forth in Atlas and Parks (eds.), 1993, The Handbook of Microbiological Media, CRC Press, Boca Raton, FL. Preferred culture conditions for a given recombinant cell may be found in the scientific literature and/or from the source of the recombinant cell such as the American Type Culture Collection (ATCC).

In cases where a gluconate dehydratase coding sequence is under the control of an inducible promoter, the inducing agent, e.g., a sugar, metal salt or antibiotic, may be added to the medium at a concentration effective to induce expression of gluconate dehydratase polypeptide.

The recombinant cell of the disclosure may comprise further genetic modifications resulting in increased production of pyruvate and/or glyceraldehyde from KDG; isopentenyl pyrophosphate (IPP) and/or dimethylallyl pyrophosphate (DMAPP) from KDG; and/or terpenoids from KDG.

Increased production of pyruvate and/or glyceraldehyde from KDG may be achieved by increasing expression of a KDG/KDPG aldolase.

Increased production of IPP or DMAPP from KDG may be achieved by increasing expression of at least one gene encoding an enzyme involved in the non-mevalonate (MEP) pathway. In certain embodiments, the at least one gene encoding an enzyme involved in the MEP pathway is selected from dxs, dxr/ispC, yghP/ispD, ychB/ispE, yghB/ispF, gcpE/ispG, and lytB/ispH.

Increased production of terpenoids from KDG may be achieved by increasing expression of at least one gene encoding an enzyme involved in the terpenoid biosynthetic pathway. In certain embodiments, the at least one gene encoding an enzyme involved in the terpenoid biosynthetic pathway is selected from ispA, crtE, crtB, crtI, crtY and crtZ.

### Methods of KDG Production

The disclosure provides a method of producing KDG, the method comprising: a) culturing a recombinant cell in a suitable culture medium, wherein the recombinant cell comprises a gene encoding a heterologous gluconate dehydratase enzyme having at least 70% sequence identity to SEQ ID NO: 1; and b) allowing expression of said gene, wherein said expression results in the production of KDG. Preferably, said production of KDG is performed at a temperature of 20-45° C.

In certain embodiments, the recombinant cell for use in a method of the disclosure comprises a gene encoding a heterologous gluconate dehydratase comprising a nucleic acid sequence having at least 70% sequence identity to SEQ ID NO: 2 or 3. In certain embodiments, the recombinant cell for use in the method comprises a gene encoding a heterologous gluconate dehydratase comprising a nucleic acid sequence having at least 70% sequence identity to SEQ ID NO: 4.

Recombinant cells of the disclosure may be cultured under suitable conditions in a liquid or solid medium. In some embodiments, recombinant cells of the disclosure undergo fermentation. Fermentation conditions include batch, fed-batch and continuous fermentation. Classical batch fermentation is a closed system, wherein the composition of the medium is not subject to artificial alterations during fermentation. In fed-batch fermentation, the substrate is added in increments as fermentation progresses. In both classical batch fermentation and batch-fed fermentation, the product(s) remain in the bioreactor until the end of the process. Batch and fed-batch fermentation are common and well-known in the art. In continuous fermentation, a defined medium is added continuously to the bioreactor and an equal volume of product containing medium is removed simultaneously. Continuous fermentation aims to maintain steady state growth conditions. Methods for modulating nutrients and growth factors for continuous fermentation processes as well as techniques for maximizing the rate of product formation are well known in the art of industrial microbiology. The fermentation process may be an aerobic or an anaerobic fermentation process.

The fermentation process is typically run at a temperature that is optimal for growth of the recombinant gluconate dehydratase-expressing cell. Fermentation is typically carried out at a temperature within the range of from about 20° C. to about 45° C., from about 25° C. to about 40° C., or from about 30° C. to about 37° C. Fermentation is typically carried out at a pH in the range of 4 to 8, in the range of 5 to 7, or the range of 5.5 to 6.5. In certain embodiments, fermentation is carried out for a period of time within the range of from about 8 to 240 hours, from about 12 hours to about 168 hours, from about 16 hours to about 144 hours, from about 20 hours to about 120 hours, from about 24 hours to about 72 hours, or from about 46 to about 48 hours.

In certain embodiments, fermentation is carried out in continuous culture. Advantageously, the method of the disclosure achieves particularly efficient and high yields of KDG because the gluconate dehydratase of the disclosure provides commercially desirable yields of KDG at mesophilic temperatures, which temperatures also permit optimal growth rates of commercially useful recombinant cells, such as E. coli.

In certain embodiments, production of KDG does not comprise a step of increasing temperature to at least 45° C. In certain embodiments, production of KDG does not comprise a step of increasing temperature to at least 48° C. In certain embodiments, production of KDG does not comprise a step of increasing temperature to at least 49° C. In certain embodiments, production of KDG does not comprise a step of increasing temperature to at least 50° C.

The culture medium may comprise naturally occurring polymers, e.g. lignocellulose, pectin, alginate, algal cell walls. The culture medium may comprise D-glucose, glycerol, D-galacturonate, L-galactonate, D-tagaturonate, D-altronate, methyl-glucuronides, D-glucuronate, D-fructuronate, D-mannonate, γ-gluconolactone, and/or D-gluconate. In certain embodiments, the culture medium comprises glycerol and glucose. In certain embodiments, glucose is added to the culture medium at the end of the log phase of cell growth. In certain embodiments, glucose is added to the culture medium at the beginning of the stationary phase of cell growth.

In certain embodiments, a method of the disclosure produces KDG at a yield of 60%. A KDG yield of at least 60% is considered to be a commercially desirable yield. As described herein, the yield of KDG is calculated as the ratio of the weight of KDG produced to the weight of substrate consumed. Weight of substrate consumed is calculated by subtracting the amount of substrate present in the supernatant at the end of the reaction from the amount of substrate present in the supernatant at the beginning of the reaction. In certain embodiments, the substrate for KDG production is glucose. Thus, in certain embodiments, at least 0.6 g of KDG is produced for every 1 g of glucose consumed by the recombinant cell. In certain embodiments, the yield of KDG is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. KDG concentration (or weight) may be determined using conventional methods known in the art, e.g. column chromatography (see e.g. US 7,125,704). KDG concentration (or weight) may be determined using ion chromatography.

In certain embodiments, a method of the disclosure comprises purifying KDG from cell culture to provide purified KDG. The method of the disclosure may comprise purifying KDG from culture supernatant to provide purified KDG.

In certain embodiments, a method of the disclosure comprises harvesting and lysing recombinant cells to release intracellular KDG from the recombinant cell. The method of the disclosure may further comprise purifying KDG from the lysate to provide purified KDG.

The disclosure also provides a cell-free method for producing KDG comprising incubating a gluconate dehydratase polypeptide of the disclosure with D-gluconate. Typically, a cell-free method of the disclosure involves incubating the gluconate dehydratase polypeptide of the disclosure under suitable conditions of temperature, pH, and ionic strength with a suitable substrate, e.g. D-gluconate. A cell-free method of the disclosure may comprise use of an immobilized gluconate dehydratase polypeptide of the disclosure. In certain embodiments, an immobilized gluconate dehydratase polypeptide of the disclosure is immobilized on an inert, insoluble substrate. An immobilized gluconate dehydratase polypeptide may have greater stability compared with a soluble form and may be more readily recovered from reaction mixtures. A cell-free method of the disclosure may further comprise purifying KDG from the cell-free reaction mixture to provide purified KDG.

Wherein the KDG was produced in a recombinant cell of the disclosure, purifying the KDG typically comprises separating KDG from components of the culture medium, fermentation process, and/or cellular material. Wherein the KDG was produced in a cell-free method, purifying the KDG typically comprises separating KDG from the cell-free reaction mixture. KDG may be purified using conventional methods known in the art.

The method of the disclosure may further comprise converting KDG to a 2′-deoxynucleoside or a precursor thereof. Methods for converting KDG to 2′-deoxynucleoside are known in the art, e.g. see WO2004/113358. KDG is converted to 2′-deoxynucleoside by a decarboxylation reaction catalyzed by a keto acid decarboxylase. In certain embodiments, KDG is converted to 2-deoxy-D-ribose which may be used as a starting substrate for the production of anticancer and antiviral drugs. In certain embodiments, the recombinant cell of the disclosure expresses keto acid decarboxylase. In certain embodiments, KDG is converted to a 2′-deoxynucleoside in vitro.

In certain embodiments, the method of the disclosure further comprises converting KDG to 5-hydroxymethyl-2-furoic acid (HMFA) and/or furan dicarboxylic acid (FDCA). Methods of producing HMFA and FDCA from KDG are known in the art, e.g. see WO2016/141148 and WO2017/030668. Briefly, KDG may be dehydrated in the presence of a dehydration catalyst, e.g. an acid catalyst, to synthesize HMFA. KDG may also be dehydrated to form other FDCA precursors, e.g. 2-formyl-5-furan carboxylic acid. These FDCA precursors, including HMFA, can then be oxidized to produce FDCA.

Methods of the disclosure typically comprise culturing a recombinant cell of the disclosure, as described herein.

The recombinant cell for use in a method of the disclosure may comprise genetic modifications resulting in one or more of the following phenotypes: (a) increased production of pyruvate and/or glyceraldehyde-3-phosphate from KDG; (b) increased production of isopentenyl pyrophosphate (IPP) and/or dimethylallyl pyrophosphate (DMAPP) from KDG; and (c) increased production of terpenoids from KDG.

Increased production of pyruvate and/or glyceraldehyde from KDG may be achieved by increasing the expression of at least one gene encoding a KDG/KDPG aldolase.

Increased production of IPP and/or DMAPP from KDG may be achieved by increasing expression of at least one gene encoding an enzyme involved in the non-mevalonate (MEP) pathway, e.g. increasing expression of DOXP synthase (dxs), DOXP reductoisomerase (dxr/ispC), CDP-ME synthase (yghP/ispD), CPD-ME kinase (ychB/ispE), MEcPP synthase (yghB/ispF), HMB-PP synthase (gcpE/ispG), isopentenyl-diphosphate Δ-isomerase (idi/ygfV) and/or HMB-PP reductase (lytB/ispH).

Increased production of terpenoids from KDG may be achieved by increasing expression of enzymes involved in the terpenoid biosynthetic pathway. Increased production of terpenoids may include increased production of geranyl pyrophosphate, geraniol, monoterpenes (e.g. 3-carene, limonene, linalool, α-pinene, terpinene), farnesyl pyrophosphate, farnesol, triterpenes (e.g. squalene), sesquiterpenes (e.g. artemisinic acid, amorphadiene, valencene, nootkatone, α-aantalene, bisabolene, patchoulol, cubebol), geranylgeranyl pyrophosphate, diterpenes (e.g. miltiradiene, casbene, texadiene), tetraterpenes (e.g. astaxanthin, β-carotene, lycopene). In certain embodiments, the recombinant cell for use in a method of the disclosure further comprises genetic modifications resulting in increased production of P-carotene.

The method of the disclosure may comprise a step of purifying from cell culture: (a) pyruvate and/or glyceraldehyde-3-phosphate; (b) IPP or DMAPP; and/or (c) terpenoids, including isoprene (or hemiterpene).

The method of the disclosure may comprise a step of purifying from supernatant: (a) pyruvate and/or glyceraldehyde-3-phosphate; (b) IPP or DMAPP; and/or (c) terpenoids.

The method of the disclosure may comprise harvesting and lysing recombinant cells to obtain: (a) pyruvate and/or glyceraldehyde-3-phosphate; (b) IPP or DMAPP; and/or (c) terpenoids.

The method of the disclosure may comprise harvesting recombinant cells and extracting them with one or more solvent(s) to obtain: (a) pyruvate and/or glyceraldehyde-3-phosphate; (b) IPP or DMAPP; and/or (c) terpenoids.

The method of the disclosure may comprise a step of purifying from lysate: (a) pyruvate and/or glyceraldehyde-3-phosphate; (b) IPP or DMAPP; and/or (c) terpenoids.

In certain embodiments, the method further comprises harvesting and extracting the cells with a solvent to obtain: (a) pyruvate and/or glyceraldehyde-3-phosphate; (b) IPP or DMAPP; and/or (c) terpenoids. In certain embodiments the solvent is an organic solvent, and in certain embodiments the solvent is immiscible in water, and in some embodiments, the solvent is an organic solvent that is immiscible in water.

The following examples are provided for the purpose of illustrating, not limiting, the material disclosed herein.

### EXAMPLES

### General Methods

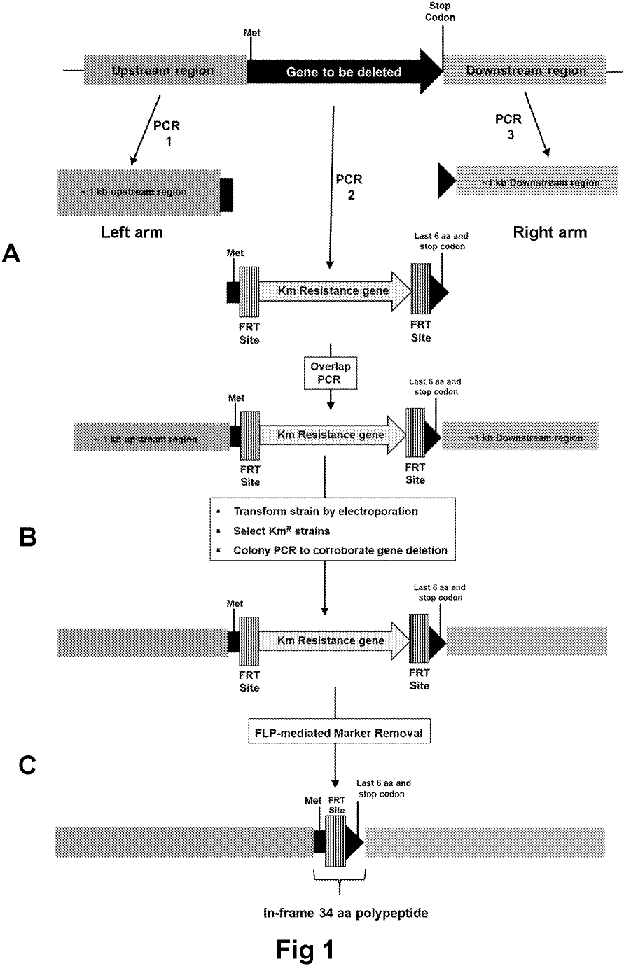

To develop a strain of E.coli for enhanced production of KDG, several genetic modifications were created in strain W3110K (CGSC 7167, Coli Generic Center, Yale University, New Haven CT). For such a purpose, the methods and tools reported by Datsenko and Wanner (2000), and Baba et al, (2006), were utilized with some small variations (see FIG. 1). Briefly, the general protocol was as follows: a kanamycin (Km) mutagenic cassette was generated by overlapping PCR (step A, FIG. 1). The structure of this cassette was designed to follow the design reported by Baba et al. (2006), which allows in-frame deletions and marker removal by FLP-mediated excision. The cassette was transformed into the strain by electroporation, and Km^{R} colonies selected. The two regions of homology flanking the Km^{R} marker enable homologous recombination with the chromosome, and replacement of the targeted gene by the Km^{R} marker (step B. FIG. 1). Once the proper replacement was confirmed by colony PCR, plasmid pCP20 (Datsenko and Wanner., ibid) was utilized to remove the Km^{R} marker (step C, FIG. 1).

All strains were stored at -70° C. in 2.0 ml frozen vials prepared with 0.3 ml of sterile 80% glycerol (Teknova Inc. Hollister CA), and 1.2 ml of the strain, grown for 18 to 20 h in LB broth, containing the necessary antibiotics.

PCR reactions were carried out using CloneAmp HiFi PCR premix, according to the recommendations of the manufacturer (Clontech/Takara Bio, Mountain View, CA).

Colony PCRs were carried out using OneTaq HS Quick-Load enzyme, according to the recommendations of the manufacturer (New England Biolabs, Ipswich, MA).

DNA sequences of all the E.coli genes used in this application were obtained from the EcoCyc E. coli Database (https://ecocyc.org/), using the W3110 genome.

KDG, glycerol, glucose and gluconate were quantified in culture supernatants using a Dionex ICS-5000 Ion Chromatography System, configured with a Pulsed Amperometric Detector. The instrument was equipped with a Dionex CarboPac PA200 column and a PA200 guard column. The following solvents were prepared and used isocratically: 100 mM NaOH in Milli Q Water (Solvent A) and 100 mM NaOH/1M NaOAc in Milli Q Water (Solvent B). The system was run isocratically at 8% Solvent B for 7 minutes. The instrument is configured with a pulsed amperometric detector. The electrochemical detector utilizes a gold electrode. The detector compartment was set to 30° C. and the column compartment to 35° C. Stock solutions in water were prepared from solid standards of glucose, gluconate and KDG, and a liquid glycerol stock. These stock solutions were subsequently diluted further while being combined into a single vial (mixture of all standards) to form a stock concentration of 5 mM for each compound. The single vial containing 5 mM of each compound was diluted with water to yield the following concentrations: 5, 2.5, 1.25, 0.625, 0.313, 0.156 and 0.078 mM. Injection volume was 10 µL for all samples and standards. All fermentation samples were diluted 1:100 in water prior to injection. Baseline separated peaks for glycerol (RT = 1.85 min), glucose (RT = 2.04 min), gluconate (RT = 2.7 min) and KDG (RT = 3.28 min). Peak integration was carried out by applying a software specific algorithm. Sample concentrations were determined based on standard curve fit.

TABLE 1

Primer sequences

| SEQ ID NO | Primer name | Sequence |
|---|---|---|
| 9 | Keio-ptsH-F1 | CTAGACTTTAGTTCCACAACACTAAACCTATAAGTTGGGGAAATACAA TG |
| 10 | Keio-crr-R1 | ATGGGCGCCATTTTTCACTGCGGCAAGAATTACTTCTTGATGCGGATA AC |
| 11 | Keio-ptsH-revF1 | CCCCAACTTATAGGTTTAGTGTTGTGG |
| 12 | PtsH-F2 | CCTGGTGCTGACGGAAGGTGC |
| 13 | Keio-crr-revR1 | GGTTATCCGCATCAAGAAGTAATTCTTGCCGCAG |
| 14 | crr-R2 | CCCTCCAGCATGGCGTTGATGC |
| 15 | PtsH-F1 | CGCCCCTCCTGGCATTGATTCAGCC |
| 16 | crr-R1 | GTGGTGGTCACTGCCGACAGCG |
| 17 | PtsH-F3 | CGCGGCGTGCTGAAACCAGGCGTTG |
| 18 | crr-R3 | GCGGTTACGGGTATTGCCGAGC |
| 19 | Keio-gntK-F1 | ACAGTTACCCGTAACATTTTTAATTCTTGTATTGTGGGGGCACCACTT TG |
| 20 | Keio-gntk-R1 | GTTAAAACAAGCGTTAATGTAGTCACTACTTATTTGCCTTTTTTAATA AC |
| 21 | Rev-Keio-gntK-F1 | CAAAGTGGTGCCCCCACAATACAAG |
| 22 | gntR-F2 | CGCCACCAGCCGGGCGATTGGCGTCC |
| 23 | Rev-Keio-gntk-R1 | GGCAAATAAGTAGTGACTACATTAACGC |
| 24 | gntU-R2 | CCCCGCCGGTTGCAGCGCGTGACCGC |
| 25 | gntR-F1 | GGCGCTGAACGCCTGCTGGCGCG |
| 26 | gntU-R1 | CCCAGGGCGACAACCACCGCCAGG |
| 27 | gntR-F3 | CGCTCTACGCGGCAAGATTGCCGCGGC |
| 28 | gntU-R3 | CGCTTCGCCCAGTGCCGGACCTACGCC |
| 29 | Keio-idnK-F1 | CGCCAGGCGTAGTATCGCAGCAGGTAAGATGATTCAGGAGATTTTAAA TG |
| 30 | Keio-idnK-R1 | GCGTGGTGTTGAAAGCCGATTTTTGAAAATCATTCGCAGCGCTGATCT GA |
| 31 | Rev-Keio-idnK-F1 | CTCCTGAATCATCTTACCTGCTGCG |
| 32 | idnD-F2 | GCACATCCGCCCCCATCTCTTTGCCC |
| 33 | Rev-Keio-idnK-R1 | CAAAAATCGGCTTTCAACACCACGC |
| 34 | ahr-R2 | GGCCACAAGATGTTGAAGTGCAGGTGG |
| 35 | idnD-F1 | CCTCACTGGTCAGGTAGACACCTCGG |
| 36 | ahr-R1 | CCGGCACGCCTTATGAGCTGCGTAAGC |
| 37 | idnD-F3 | GCCATCGCGCCTCCCATACCTACCTGC |
| 38 | ahr-R3 | CCAGGCACCCCGCCCTGCCATGCTC |
| 39 | Keio-glk-F1 | TACAGTGTGAGAAAGAATTATTTTGACTTTAGCGGAGCAGTTGAAGAA TG |
| 40 | Keio-glk-R1 | GAGTTACCTCCCGATATAAAAGGAAGGATTTACAGAATGTGACCTAAG GT |
| 41 | Rev-Keio-glk-F1 | CATTCTTCAACTGCTCCGCTAAAGTC |
| 42 | yfeO-F2 | CGCGTGCAACCGTGGTAAGCACC |
| 43 | Rev-Keio-glk-R1 | CCTTCCTTTTATATCGGGAGGTAACTCTCCCG |
| 44 | fryC-R2 | CAGCAGTTGCGTCGGCTGGGTAG |
| 45 | glk-verif-F1 | GGTTCTGGCTCGCGGATGGAGC |
| 46 | glk-verif-R1 | GGCCCATCGGGCAGGCACATAAGGC |
| 47 | yfeO-F1 | GCCACCATCTGCTGCATCTCATCC |
| 48 | fryC-R1 | GGTGGACGGCACCGGCGAAGAGG |
| 49 | kdgK-Keio-F1 | CCACAGCGCAAACTAACGCTAATTTTTTACAGATCAGGTTCACGACTA TG |
| 50 | kdgK-Keio-R1 | CTTTATCCAGCCTTTTGCATATGCTGCGTTTACGCTGGCATCGCCTCA CG |
| 51 | Rev-F-kdgK-Keio | CATAGTCGTGAACCTGATCTGTAAAAAATTAGCGTTAGTTTGCGCTGT GG |
| 52 | pdeH-F1 | GCCTTGTGCGGCGAATGCGGGCGAG |
| 53 | Rev-R-kdgK-Keio | CGTGAGGCGATGCCAGCGTAAACGCAGCATATGCAAAAGGCTGG |
| 54 | yhjJ-R1 | CACCCCGGATGCAATGACGCTACTGG |
| 55 | kdgK-verif-F1 | CCTCACTCCACAACGGAATCTTTCAGG |
| 56 | kdgK-verif-R1 | GCGACCACCTCTGTCGCCACGGATGACG |
| 57 | pdeH-F2 | GCGCCAGAACCGCCGTATTCAGCG |
| 58 | yhj J-R2 | GGTCACGATCCTGCCGATCCGCTG |
| 59 | gudD-F1 | ATGAGTTCTCAATTTACGACGCCTG |
| 60 | gudD-R1 | TTAACGCACCATGCACGGGCGCTTG |
| 61 | gudD-F2 | GAGGAATAAACCATGAGTTCTCAATTTACGACGCCTG |
| 62 | gudD-R2 | TTGTTCTAGAAAGCTTAACGCACCATGCACGGGCGCTTG |
| 63 | BP2-F1 | GTGTGGGCACTCGACCGGAATTATCG |
| 64 | BP2-R1 | GGTCGACGGCGCTATTCAGATCC |
| 65 | E.coli gudX-F1 | ATGGCGACACAATCCAGTCCTG |
| 66 | E.coli gudX-R1 | TTAATGACGGCCGAAAACGGGACGTTTACGG |
| 67 | E.coli gudX-F2 | GAGGAATAAACCATGGCGACACAATCCAGTCCTG |
| 68 | E.coli gudX-R2 | TTGTTCTAGAAAGCTTAATGACGGCCGAAAACGGGACGTTTACGG |
| 69 | uxuA-F1 | ATGGAACAGACCTGGCGCTGG |
| 70 | uxuA-R1 | TTAACGGCTAAAGAAAGCGCGCTGG |
| 71 | uxuA-F2 | GAGGAATAAACCATGGAACAGACCTGGCGCTGG |
| 72 | uxuA-R2 | TTGTTCTAGAAAGCTTAACGGCTAAAGAAAGCGCGCTGG |
| 73 | rspA-F1 | ATGAAGATCGTAAAGGCTGAAGTT |
| 74 | rspA-R1 | TTACCAGTTCCACAGCGTGCCATCTTCC |
| 75 | rspA-F2 | GAGGAATAAACCATGAAGATCGTAAAGGCTGAAGTT |
| 76 | rspA-R2 | TTGTTCTAGAAAGCTTACCAGTTCCACAGCGTGCCATCTTCC |
| 77 | Achro-DHT-F1 | GAGGAATAAACCATGTCGCAAACTCCTCGCAAACTGCG |
| 78 | Achro-DHT-R1 | TTGTTCTAGAAAGCTTAGTGAGAGTGACGCGGATCACCACG |
| 80 | crtE-F2 | GAGGAATTAACCATGACGGTCTGCGCAAAAAAACACGTTCATC |
| 81 | crtB-R2 | GTTCGGGCCCAAGCTTATTAGAGCGGGCGCTGCCAGAGATGCGC |

### Example 1 Deletion of the ptsH-ptsI-crr Genes: Strain Δ1

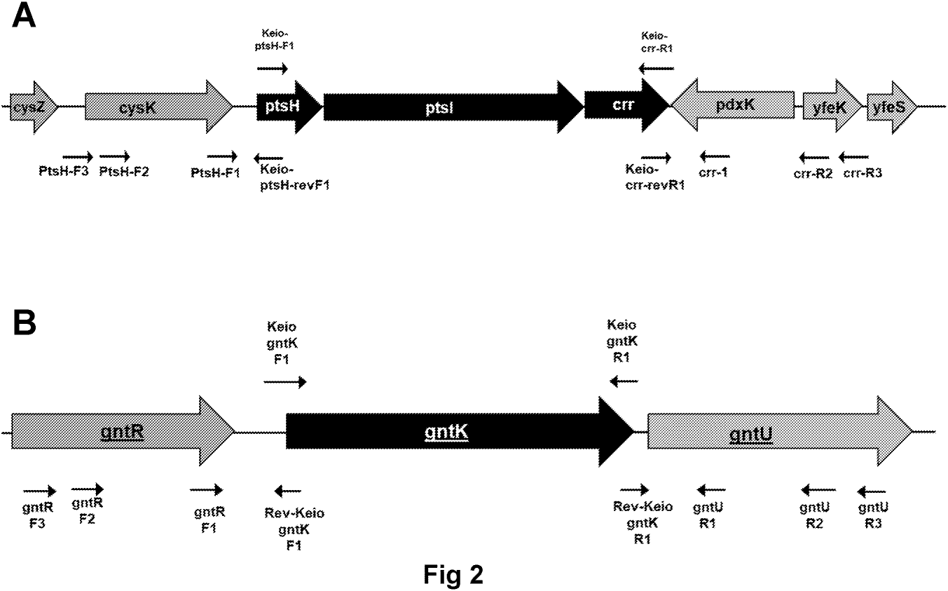

Glucose is predominantly taken up into E. coli by the PEP-dependent phosphotransferase system, three components of which are encoded by ptsH, ptsI and crr. These three genes are contiguous in the chromosome. Locations of all the primers used to delete them are shown in FIG. 2A and their sequences are presented in Table 1. Primers Keio-ptsH-F1 (SEQ ID NO: 9) and Keio-crr-R1 (SEQ ID NO: 10) were used to amplify the Km^{R} gene from pKD13 (Datsenko and Wanner., ibid). Primers Keio-ptsH-revF1 (SEQ ID NO: 11) and ptsH-F2 (SEQ ID NO: 12) were utilized to amplify the left arm for the mutagenic cassette. Primers Keio-crr-revR1 (SEQ ID NO: 13) and crr-R2 (SEQ ID NO: 14) were used to obtain the right arm. These 3 PCR fragments were gel-purified and utilized for an overlap PCR reaction, using the ptsH-F2 (SEQ ID NO: 12) and crr-R2 (SEQ ID NO: 14) primers to obtain the full-length mutagenic primer. Electrocompetent cells of W3110K were prepared and transformed with the purified full-length mutagenic cassette. Colony PCR with primers PtsH-F1 (SEQ ID NO: 15) and crr-R1 (SEQ ID NO: 16) was used to identify colonies where the proper deletion has occurred. One colony with the correct deletion was selected as the W3110k ΔptsH-ptsI-crr::Km strain. For short, this strain was named Δ1-Km. To remove the Km marker from this strain, plasmid pCP20 was transformed and the protocol described by Datsenko and Wanner (ibid) was followed. Colonies that were Km^{S}, Carb^{S} were analyzed by colony-PCR utilizing primers PtsH-F3 (SEQ ID NO: 17) and crr-R3 (SEQ ID NO: 18). A colony that produced the correct PCR product was chosen and named W3110K ΔptsH-ptsI-crr-no marker, or for short: Δ1.

### Example 2 Deletion of the gntK Gene: Strain Δ2

The conversion of gluconate to gluconate-6-phosphate may be catalyzed by a gluconate kinase encoded by gntK. As shown in FIG. 2B, the gntK gene is in an operon with the gntU gene. Locations of all the primers used to delete gntK are shown in FIG. 2B and their sequences are presented in Table 1. Primers Keio-gntK-F1 (SEQ ID NO: 19) and Keio-gntK-R1 (SEQ ID NO: 20) were used to amplify the Km^{R} gene from pKD13 (Datsenko and Wanner., ibid). Primers Rev-Keio-gntK-F1 (SEQ ID NO: 21) and gntR-F2 (SEQ ID NO: 22) were utilized to amplify the left arm for the mutagenic cassette. Primers Rev-Keio-gntK-R1 (SEQ ID NO: 23) and gntU-R2 (SEQ ID NO: 24) were used to obtain the right arm. These 3 PCR fragments were gel-purified and utilized for an overlap PCR reaction, using the gntR-F2 (SEQ ID NO: 22) and gntU-R2 (SEQ ID NO: 24) primers to obtain the full-length mutagenic primer. Electrocompetent cells of strain Δ1 (described in example 1) were prepared and transformed with the purified full-length mutagenic cassette. Colony PCR with primers gntR-F1 (SEQ ID NO: 25) and gntU-R1 (SEQ ID NO: 26) was used to identify colonies where the proper deletion has occurred. One colony with the correct deletion was selected as the W3110k ΔptsH-ptsI-crr, ΔgntK::Km strain. For short, this strain was named Δ2-Km. To remove the Km marker from this strain, plasmid pCP20 was transformed and the protocol described by Datsenko and Wanner (ibid) was followed. Colonies that were Km^{S}, Carb^{S} were analyzed by colony-PCR utilizing primers gntR-F3 (SEQ ID NO: 27) and gntU-R3 (SEQ ID NO: 28). A colony that produced the correct PCR product was chosen and named W3110K ΔptsH-ptsI-crr, ΔgntK-no marker, or for short: Δ2.

### Example 3 Deletion of the idnK Gene: Strain Δ3

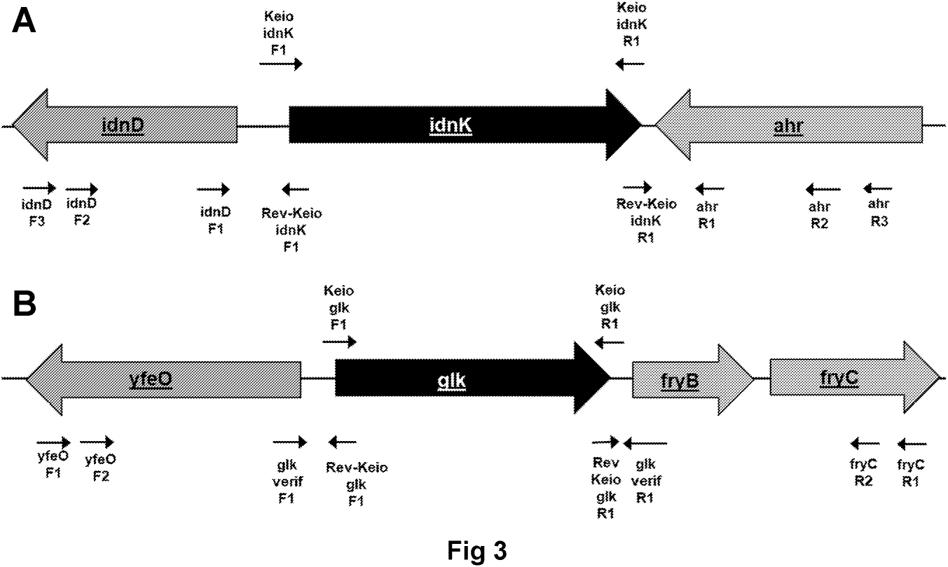

The conversion of gluconate to gluconate-6-phosphate may also be catalyzed by a gluconate kinase encoded by idnK. As shown in FIG. 3A, the idnK gene is in a single transcriptional unit. Locations of all the primers used to delete idnK, are shown in FIG. 3A and their sequences are presented in Table 1. Primers Keio-idnK-F1 (SEQ ID NO: 29) and Keio-idnK-R1 (SEQ ID NO: 30) were used to amplify the Km^{R} gene from pKD13 (Datsenko and Wanner., ibid). Primers Rev-Keio-idnK-F1 (SEQ ID NO: 31) and idnD-F2 (SEQ ID NO: 32) were utilized to amplify the left arm for the mutagenic cassette. Primers Rev-Keio-idnK-R1 (SEQ ID NO: 33) and ahr-R2 (SEQ ID NO: 34) were used to obtain the right arm. These 3 PCR fragments were gel-purified and utilized for an overlap PCR reaction, using the idnD-F2 (SEQ ID NO: 32) and ahr-R2 (SEQ ID NO: 34) primers to obtain the full-length mutagenic primer. Electrocompetent cells of strain Δ2 (described in example 2) were prepared and transformed with the purified full-length mutagenic cassette. Colony PCR with primers idnD-F1 (SEQ ID NO: 35) and ahr-R1 (SEQ ID NO: 36) was used to identify colonies where the proper deletion has occurred. One colony with the correct deletion was selected as the W3110k ΔptsH-ptsI-crr, ΔgntK, ΔidnK::Km strain. For short, this strain was named Δ3-Km. To remove the Km marker from this strain, plasmid pCP20 was transformed and the protocol described by Datsenko and Wanner (ibid) was followed. Colonies that were Km^{S}, Carb^{S} were analyzed by colony-PCR utilizing primers idnD-F3 (SEQ ID NO: 37) and ahr-R3 (SEQ ID NO: 38). A colony that produced the correct PCR product was chosen and named W3110K ΔptsH-ptsI-crr, ΔgntK, ΔidnK-no marker, or for short: Δ3.

### Example 4 Deletion of the Glk Gene: Strain Δ4

The conversion of glucose to glucose-6-phosphate is catalyzed by a glucokinase encoded by glk. As shown in FIG. 3B, the glk gene is in a single transcriptional unit. Locations of all the primers used to delete glk are shown in FIG. 3B and their sequences are presented in Table 1. Primers Keio-glk-F1 (SEQ ID NO: 39) and Keio-glk-R1 (SEQ ID NO: 40) were used to amplify the Km^{R} gene from pKD13 (Datsenko and Wanner., ibid). Primers Rev-Keio-glk-F1 (SEQ ID NO: 41) and yfeO-F2 (SEQ ID NO: 42) were utilized to amplify the left arm for the mutagenic cassette. Primers Rev-Keio-glk-R1 (SEQ ID NO: 43) and fryC-R2 (SEQ ID NO: 44) were used to obtain the right arm. These 3 PCR fragments were gel-purified and utilized for an overlap PCR reaction, using the yfeO-F2 (SEQ ID NO: 42) and fryC-R2 (SEQ ID NO: 44) primers to obtain the full-length mutagenic primer. Electrocompetent cells of strain Δ3 (described in example 3) were prepared and transformed with the purified full-length mutagenic cassette. Colony PCR with primers glk-verif-F1 (SEQ ID NO: 45) and glk-verif-R1 (SEQ ID NO: 46) was used to identify colonies where the proper deletion has occurred. One colony with the correct deletion was selected as the W3110k ΔptsH-ptsI-crr, ΔgntK, ΔidnK, Δglk::Km strain. For short, this strain was named Δ4-Km. To remove the Km marker from this strain, plasmid pCP20 was transformed and the protocol described by Datsenko and Wanner (ibid) was followed. Colonies that were Km^{S}, Carb^{S} were analyzed by colony-PCR utilizing primers yfeO-F1 (SEQ ID NO: 47) and fryC-R1 (SEQ ID NO: 48). A colony that produced the correct PCR product was chosen and named W3110K ΔptsH-ptsI-crr, ΔgntK, ΔidnK, Δglk-no marker, or for short: Δ4.

### Example 5 Deletion of the kdgK Gene: Strain Δ5

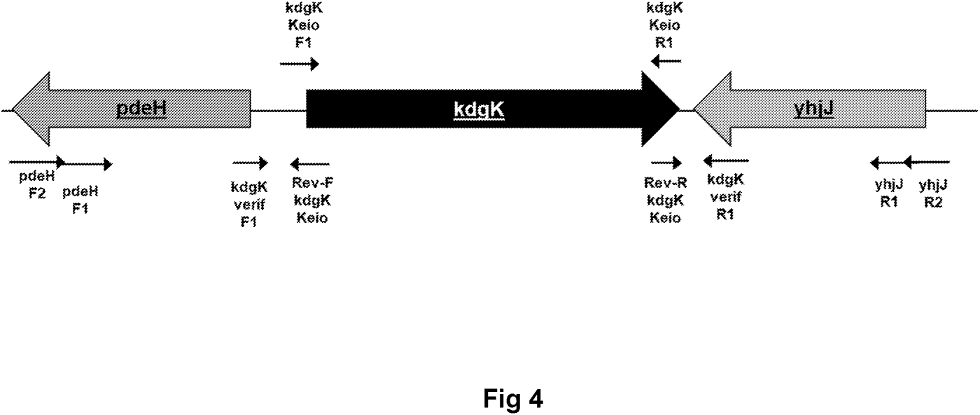

The phosphorylation of KDG is catalyzed by a 2-dehydro-3-deoxygluconokinase encoded by kdgK. As shown in FIG. 4, the kdgK gene is in a single transcriptional unit. Locations of all the primers used to delete kdgK are shown in FIG. 4 and their sequences are presented in Table 1. Primers kdgK-Keio-F1 (SEQ ID NO: 49) and kdgK-Keio-R1 (SEQ ID NO: 50) were used to amplify the Km^{R} gene from pKD13 (Datsenko and Wanner., ibid). Primers Rev-F-kdgK-Keio (SEQ ID NO: 51) and pdeH-F1 (SEQ ID NO: 52) were utilized to amplify the left arm for the mutagenic cassette. Primers Rev-R-kdgK-Keio (SEQ ID NO: 53) and yhjJ-R1 (SEQ ID NO: 54) were used to obtain the right arm. These 3 PCR fragments were gel-purified and utilized for an overlap PCR reaction, using the pdeH-F1 (SEQ ID NO: 52) and yhjJ-R1 (SEQ ID NO: 54) primers to obtain the full-length mutagenic primer. Electrocompetent cells of strain Δ4 (described in example 4) were prepared and transformed with the purified full-length mutagenic cassette. Colony PCR with primers kdgK-verif-F1 (SEQ ID NO: 55) and kdgK-verif-R1 (SEQ ID NO: 56) was used to identify colonies where the proper deletion has occurred. One colony with the correct deletion was selected as the W3110k ΔptsH-ptsI-crr, ΔgntK, ΔidnK, Δglk, ΔkdgK::Km strain. For short, this strain was named A5-Km. To remove the Km marker from this strain, plasmid pCP20 was transformed and the protocol described by Datsenko and Wanner (ibid) was followed. Colonies that were Km^{S}, Carb^{S} were analyzed by colony-PCR utilizing primers pdeH-F2 (SEQ ID NO: 57) and yhjJ-R2 (SEQ ID NO: 58). A colony that produced the correct PCR product was chosen and named W3110K ΔptsH-ptsI-crr, ΔgntK, ΔidnK, Δglk, ΔkdgK-no marker, or for short: Δ5.

### Example 6 Cloning of the E.coli gudD Gene

To assess the ability of endogenous E. coli sugar dehydratases to produce KDG from gluconate the inventors expressed two D-glucarate dehydratases (gudD and gudX), mannonate dehydratase (uxuA), D-galactonate dehydratase (rspA) in the recombinant Δ3 E. coli strain.

To over-express sugar dehydratase genes in E.coli, the expression vector pTrc-His2c (Invitrogen, Carlsbad, CA) was chosen. Thisvector was digested with restriction enzymes NcoI and HindIII. Linear digested DNA was gel-purified.

To clone the E.coli gudD into the linear pTrc-His2c vector, the gene was amplified by PCR using chromosomal DNA from strain MG1655; first using primers gudD-F1 (SEQ ID NO: 59) and gudD-R1 (SEQ ID NO: 60). The obtained PCR product was column-purified. Second, primers gudD-F2 (SEQ ID NO: 61) and gudD-R2 (SEQ ID NO: 62) were utilized to re-amplify the PCR product obtained in the first step. These new primers add the 15-bp complementary regions to the NcoI-HindIII digested pTrc-His2 vector, which are needed to clone using the In-Fusion cloning kit (Takara Bio, Mountain View, CA). The PCR product was column-purified and the In-Fusion cloning protocol was used, following the recommendations of the provider. Primers BP2-F1 (SEQ ID NO: 63) and BP2-R1 (SEQ ID NO: 64) were used to identify the correct clones by colony PCR. Two clones that showed the correct insert size were corroborated by DNA sequencing. One of the clones was named pTrc-GudD1 and used for subsequent experiments.

### Example 7 Cloning of the E.coli gudXGene

The procedure to clone the E.coli gudX gene into the expression vector pTrc-His2c was the same as described in Example 6, with the following differences:

First, the E.coli gudX gene was amplified by PCR using primers gudX-F1 (SEQ ID NO: 65) and gudX-R1 (SEQ ID NO: 66). Primers gudX-F2 (SEQ ID NO: 67) and gudX-R2 (SEQ ID NO: 68) were utilized to add the In-Fusion cloning ends. The clone named pTrc-GudX5 was used for subsequent experiments.

### Example 8 Cloning of the E.coli uxuA Gene

The procedure to clone the E.coli uxuA gene into the expression vector pTrc-His2c was the same as described in Example 6, with the following modifications:

First the E.coli uxuA gene was amplified by PCR using primers uxuA-F1 (SEQ ID NO: 69) and uxuA-R1 (SEQ ID NO: 70). Primers uxuA-F2 (SEQ ID NO: 71) and uxuA-R2 (SEQ ID NO: 72) were utilized to add the In-Fusion cloning ends. The clone named pTrc-UxuA10 was used for subsequent experiments.

### Example 9 Cloning of the E.coli rspA Gene

The procedure to clone the E.coli rspA gene into the expression vector pTrc-His2c was the same as described in Example 6, with the following modifications:

First the E.coli rspA gene was amplified by PCR using primers rspA-F1 (SEQ ID NO: 73) and rspA-R1 (SEQ ID NO: 74). Primers rspA-F2 (SEQ ID NO: 75) and rspA-R2 (SEQ ID NO: 76) were utilized to add the In-Fusion cloning ends. The clone named pTrc-RspA4 was used for subsequent experiments.

### Example 10 Cloning of the Achromobacter Sp. Gluconate Dehydratase

To explore whether GenBank entry WP_054458272 (SEQ ID NO: 1), annotated as a putative dihydroxy-acid dehydratase (DHT) from Achromobacter sp. could function as a gluconate dehydratase, an E.coli codon-optimized synthetic gene (SEQ ID NO: 4) was synthesized by IDT (San Diego, CA). The gene was delivered cloned into the pUC-Km vector. This clone was used as a template to amplify by PCR, the DHT gene for cloning into pTrc-His2c (SEQ ID NO: 5) as described in Example 6. Primers Achro-DHT-F1 (SEQ ID NO: 79) and Achro-DHT-R1 (SEQ ID NO: 78) were utilized to add the In-Fusion ends for cloning. Primers BP2-F1 (SEQ ID NO: 63) and BP2-R1 (SEQ ID NO: 64) were used to identify the correct clones by colony PCR. Two clones that showed the correct insert size were corroborated by DNA sequencing. One of the clones was named pAchro-10 (SEQ ID NO: 6) and used for subsequent experiments.

### Example 11 Growth of Δ3-Km Strain Expressing Different Sugar Dehydratases

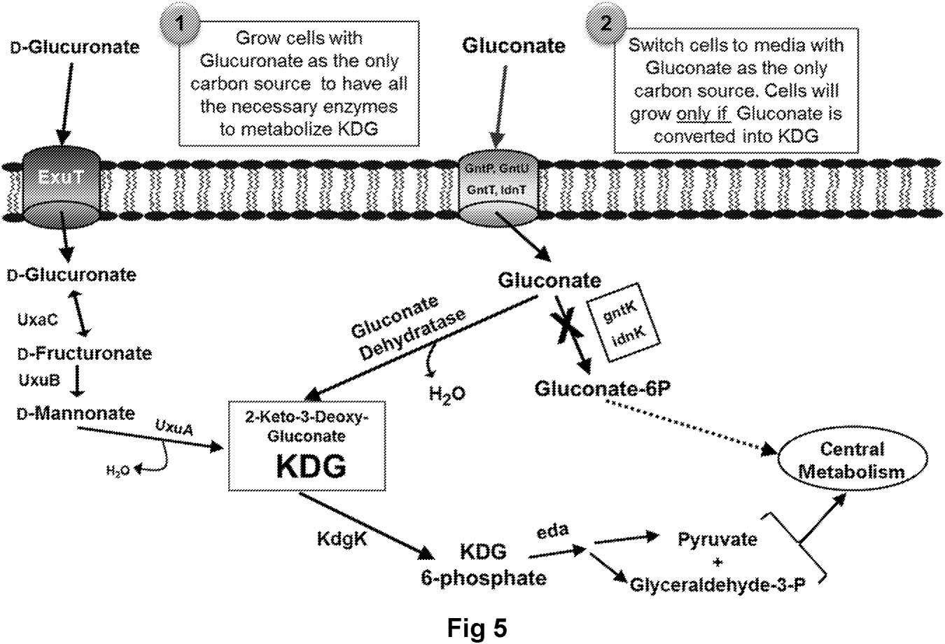

The mutations ΔgntK and ΔidnK present in the Δ3-Km strain are known to block the use of gluconate as the only carbon source (Tong S., et al. 1996). Conversion of gluconate into KDG by a gluconate dehydratase should allow growth on gluconate again, via the glucuronate-assimilation pathway which uses KDG as an intermediate (See FIG. 5). To evaluate if the Achromobacter sp. dihydroxy-acid dehydratase cloned in Example 10, as well the other sugar dehydratases cloned in Examples 6 to 9, would restore growth on gluconate, plasmids containing these dehydratases were transformed into Δ3-Km strain.

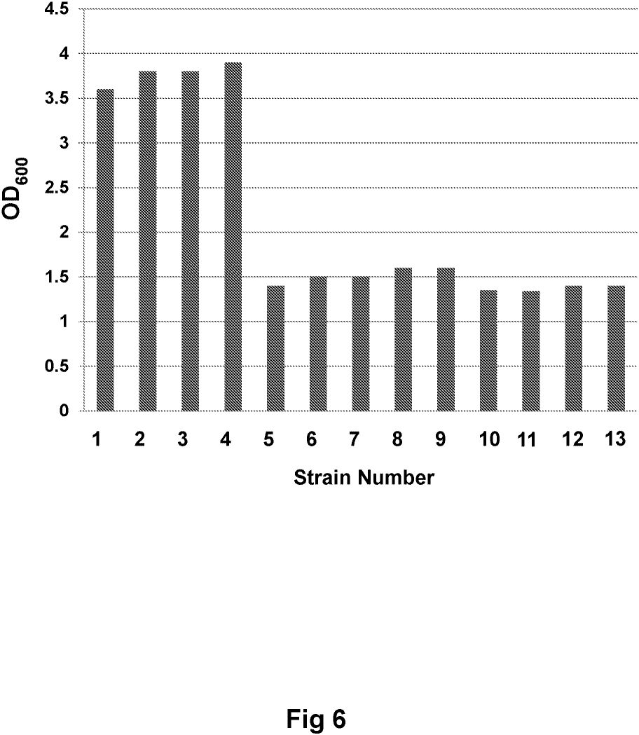

Four different isolates from Δ3-Km containing pAchro-10, and 2 independent isolates of strains containing the GudD, RspA, UxuA or GudX dehydratases were used for growth comparison experiments. Strains were grown first in 3ml of LB broth (Teknova Inc. Hollister CA ), containing 100 µg/ml carbenicillin and 50 µg/ml kanamycin. After overnight growth at 37° C., 10 µL of the cultures was used to inoculate 15 ml tubes containing 3 ml of Hi-def Azure media (Teknova Inc. Hollister CA) containing 1% glucuronate, 100 µg/ml carbenicillin and 50 µg/ml kanamycin. After 24 h growth at 37° C., 10 µL of the cultures were transferred to 15 ml tubes containing 2.5 ml of Hi-def Azure media containing 1% gluconate, 100 µg/ml carbenicillin and 50 µg/ml kanamycin. Because the promoter P_{Trc} is known to be leaky, no IPTG was used to induce expression. As a control, the Δ3-Km strain containing the empty vector pTrc-His2c was also included. Optical density of the cultures was measured after 24 hr. As shown in FIG. 6 and table 2, only strains containing pAchro-10 were able to reach high ODs, while strains containing the other 4 sugar dehydratases and the empty vector reached the same low ODs, indicating that these 4 sugars dehydratases did not allow gluconate assimilation. It should be mentioned that the Hi-def Azure media contains a small amount of amino acids, which can serve as a carbon source and allows growth up to around 1 OD.

TABLE 2

| Strain number | Plasmid | OD₆₀₀ |
|---|---|---|
| 1 | Achro-10-1 | 3.6 |
| 2 | Achro-10-2 | 3.8 |
| 3 | Achro-10-3 | 3.8 |
| 4 | Achro-10-4 | 3.9 |
| 5 | pTrc-1 | 1.3 |
| 6 | GudD-1 | 1.5 |
| 7 | GudD-2 | 1.5 |
| 8 | RspA-1 | 1.6 |
| 9 | RspA-2 | 1.6 |
| 10 | UxuA-1 | 1.3 |
| 11 | UxuA-2 | 1.3 |
| 12 | GudX5-1 | 1.4 |
| 13 | GudX5-2 | 1.4 |

### Example 12 Cloning of the Achromobacter Sp. Gluconate Dehydratase in to a Low-Copy Vector

To evaluate the in vivo efficiency of the identified Achromobacter sp. gluconate dehydratase cloned in Example 10, the E.coli codon-optimized gene (SEQ ID NO: 4) was cloned into a low-copy expression vector pBP2 (SEQ ID NO: 7). This synthetic plasmid is based on the pCL1920 (Lerner C. and Inouye M. 1990) and contains the pSC101 origin of replication, the Spectinomycin resistance marker, the P_{Trc} promoter and the lacI repressor from Ptrc-His2b, and a multicloning site. pCL-BP2 was digested with NcoI and HindIII, and the Achromobacter sp DHT cloned by In-Fusion as described in Example 10. After the DNA sequence was verified, an isolate was named pAchro-L1 (SEQ ID NO: 8) and used for subsequent experiments.

### Example 13 Comparison of pAchro-10 and pAchro-L1 in Δ3-Km Strain

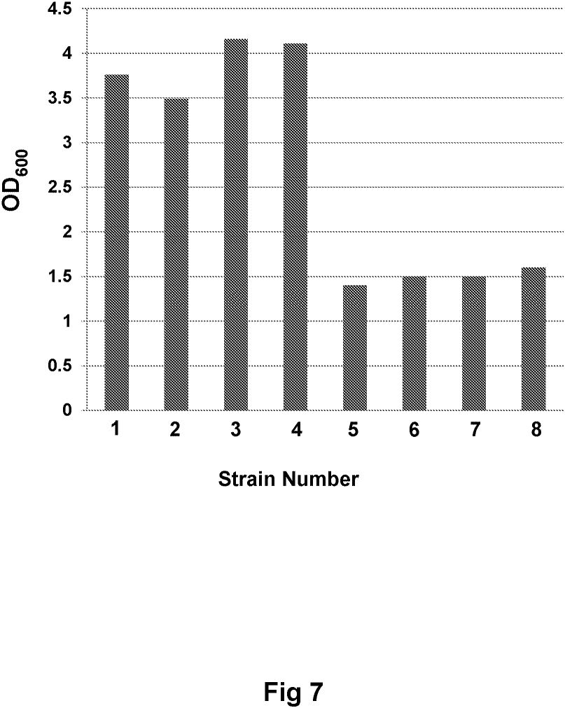

The mutations ΔgntK and ΔidnK present in the Δ3-Km strain are known to block the use of gluconate as the only carbon source (Tong S., et al. 1996). Conversion of gluconate into KDG by a gluconate dehydratase should restore the ability to grow on gluconate via the glucuronate-assimilation pathway which uses KDG as an intermediate (See FIG. 5). To evaluate whether the Achromobacter sp. dihydroxy-acid dehydratase cloned in Example 10, would restore growth on gluconate, the empty vector (pTrc-His2c), the high copy plasmid (pAchro-10) and the low copy plasmid (pAchro-L1) were transformed into the Δ3-Km strain. Two isolates from Δ3-Km containing pTrc-His2c, pAchro-10 or pAchro-L1 were used for growth comparison experiments. Strains were grown first in 3ml of LB broth (Teknova Inc. Hollister CA) containing 100 µg/ml carbenicillin and 50 µg/ml kanamycin. After overnight growth at 37° C., 10 µL of the cultures was used to inoculate 15 ml tubes containing 3 ml of Hi-def Azure media (Teknova Inc. Hollister CA) containing 1% glucuronate, 100 µg/ml carbenicillin and 50 µg/ml kanamycin. After 24 h growth at 37° C., 10 µL of the cultures were transferred to 15 ml tubes containing 2.5 ml of Hi-def Azure media containing 100 µg/ml carbenicillin and 50 µg/ml kanamycin and either 1% gluconate or no carbon source. Because the promoter P_{Trc} is known to be leaky, no IPTG was used to induce expression. Optical density of the cultures was measured after 15 hr. As shown in FIG. 7 and Table 3, the use of a low copy expression plasmid (pAchro-L1) to express the Achromobacter sp. gluconate dehydratase did not affect growth on gluconate, indicating that the gluconate dehydratase activity is very efficient and allows the recombinant cell to achieve fast growth even when expressed from a low copy number plasmid, without induction.

TABLE 3

| Strain number | Plasmid | Carbon source added | OD₆₀₀ |
|---|---|---|---|
| 1 | Achr-10-1 | Gluconate | 3.76 |
| 2 | Achr-10-2 | Gluconate | 3.49 |
| 3 | Achr-L1-1 | Gluconate | 4.16 |
| 4 | Achr-L1-2 | Gluconate | 4.11 |
| 5 | Achr-10-1 | None | 1.54 |
| 6 | Achr-10-2 | None | 1.66 |
| 7 | Achr-L1-1 | None | 1.49 |
| 8 | Achr-L1-2 | None | 1.61 |

### Example 14 Production of KDG by Resting Cells

The Δ5 E. coli strain exhibits uncoupled cell growth from KDG production. In this example, the Δ5 E. coli strain culture medium comprises glycerol as the main carbon source for cell growth. Glucose is added to the culture medium as the carbon source for KDG production. Glucose is converted to gluconate by a periplasmic glucose dehydrogenase which is then converted to KDG by the recombinant gluconate dehydratase of the disclosure. To prevent the conversion of glucose to gluconate becoming a rate limiting step, the culture medium is supplemented with pyrroloquinoline quinone (PQQ), the redox cofactor of glucose dehydrogenase.

A seed culture of E. coli strain Δ5 (described in Example 5) containing plasmid pAchro-10 (see example 10) was generated in a 2.8 L Fernbach flask containing 1x Hi-Def Azure medium (Teknova, Hollister, CA), 50 g/L glycerol, 60 µg/ml of pyrroloquinoline quinone (PQQ), 50 µg/mL kanamycin and 100 µg/mL carbenicillin. The seed culture was incubated overnight at 37° C. in an orbital shaker. The overnight culture was then centrifuged to pellet the cells. Cells were washed once with a 1x M9 salts solution and centrifuged again. The cell pellet was resuspended in 800 mL of Hi-Def Azure medium, containing 50 g/L glycerol, 20 g/L glucose, and 60 µg/mL of PQQ and 100 µg/mL carbenicillin. This resuspension was transferred into a 1 L stirred-tank bioreactor (Biostat Q Plus, Sartorius Stedim) at 37° C. and pH 7.0. The culture was agitated with Rushton impellers at 800 rpm and aerated with air at 1 vvm. The pH of the culture was controlled at 7.0 by the addition of 2 M KOH. After 24 hours, the culture was supplemented with 60 µg/mL of PQQ. After 46 hours, 2 mL samples were taken, centrifuged, and the supernatant was utilized to quantify glucose, gluconate and KDG as described above. As shown in table 4, after 46 hr most of the glucose was consumed and converted into gluconate and KDG (data shown is the average of three replicates, variation = <3%). The Δ5 E. coli strain expressing the gluconate dehydratase of the disclosure converted 86% of consumed glucose to KDG at the mesophilic temperature of 37° C. Less than 10% of consumed glucose was utilized by alternative pathways, i.e. not converted to KDG or gluconate, indicating that glucose is successfully prevented from entering the central metabolism of the cell.

TABLE 4

| Results after 46 hours | Value |
|---|---|
| KDG Titer | 16.6 g/L |
| Gluconate Titer | 0.73 g/L |
| Total Products (Gluconate + KDG) Titer | 17.3 g/L |
| Glucose consumed | 19.2 g/L |
| KDG Yield from Glucose | 0.86 g/g |
| Total Products Yield from Glucose | 0.90 g/g |

### Example 15. Production of KDG by Growing Cells

To confirm that cell growth was uncoupled from KDG production, and that this uncoupling enables useful production of KDG without the need to interrupt cell growth, the Δ5 E. coli strain was grown in medium containing glycerol as a substrate for growth, and glucose as a substrate for KDG production. Two 1.5 ml frozen vials (see General methods section) of E. coli strain Δ5 (described in Example 5) carrying plasmid pAchro-10 (see example 10), were used to inoculate 800 mL of Hi-Def Azure medium containing 20 g/L glycerol, 20 g/L glucose, 60 µg/mL of PQQ, and 100 µg/mL carbenicillin in a 1 L stirred-tank bioreactor (Biostat Q Plus, Sartorius Stedim). The culture temperature was maintained at 37° C., and its pH controlled at 7.0 by the addition of 2 M KOH. The culture was agitated with Rushton impellers at 800 rpm and aerated with air at 1 vvm. After 24 hours, the culture was supplemented with 60 µg/mL of PQQ. After 48 hours, 2 mL samples were taken, centrifuged, and the supernatant was utilized to quantify glucose, gluconate and KDG as described above. As shown in table 5, after 48 hr, 10 g/L of glucose was consumed, and converted into gluconate and KDG. 75% of consumed glucose was converted to KDG confirming that cell growth has been uncoupled from KDG production. Advantageously, the gluconate dehydratase and recombinant cell of the disclosure achieve efficient production of KDG at a mesophilic temperature.

TABLE 5

| Results after 48 hours | Value |
|---|---|
| KDG Titer | 7.5 g/L |
| Gluconate Titer | 2.5 g/L |
| Total Products Titer | 10.0 g/L |
| Glucose consumed | 10.0 g/L |
| KDG Yield from Glucose | 0.75 g/g |
| Total Product Yield from Glucose | 1.0 g/g |

In conclusion, the gluconate dehydratase enzyme and recombinant cells of the disclosure achieve efficient production of KDG at a mesophilic temperature (20-45° C.). Surprisingly, the Δ5 E. coli strain expressing the gluconate dehydratase of the disclosure achieved an 86% conversion of glucose to KDG at 37° C. Advantageously, by uncoupling cell growth from KDG production in the presence of glucose, the recombinant cells of the disclosure achieve high rates of KDG production without interrupting cell growth. This provides a significant industrial advantage as KDG can be produced in continuous cultures. In addition, the disclosure provides an efficient method for producing KDG from glucose, which is cheaper and more readily available than purified gluconate, which is the traditional substrate for KDG production.

### Example 16 Cloning of the Lycopene-Producing Genes From I

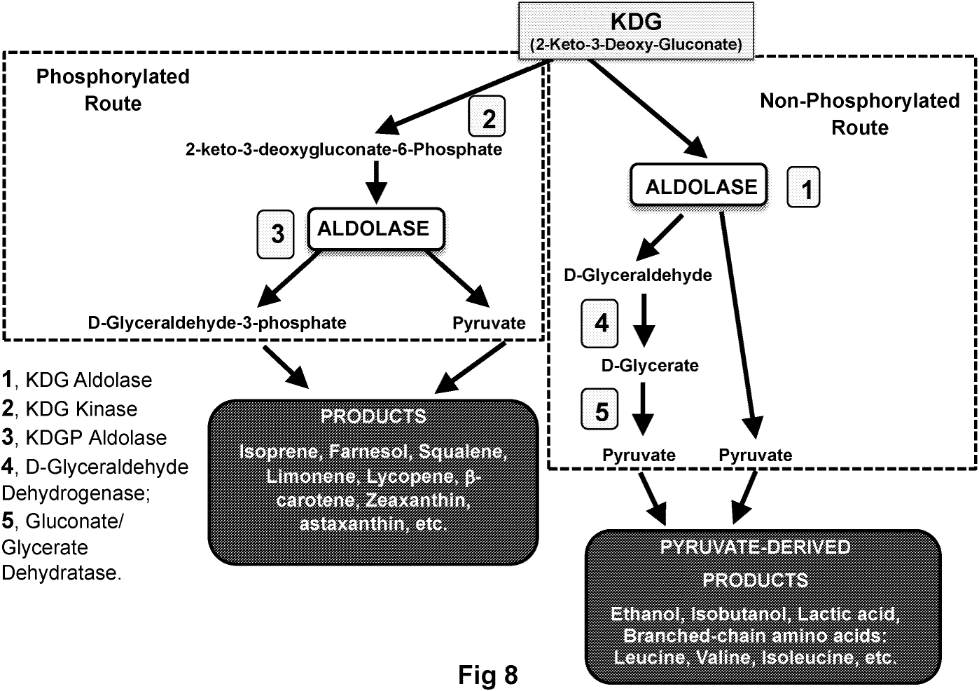

As shown in FIG. 8, KDG can be used as an intermediate to obtain other products. There are at least two different pathways to metabolize KDG: A Phosphorylated route which leads to the formation of Glyceraldehyde-2-Phosphate and Pyruvate; and a Non-phosphorylated route which produces two molecules of Pyruvate. Conversion of KDG into Pyruvate via the Non-phosphorylated route has been demonstrated in an in-vitro system, utilizing a thermophilic Gluconate dehydrogenase capable of dehydrating not only Gluconate, but also Glycerate to Pyruvate (Guterl et al., 2012 ibid).

To demonstrate that KDG can be converted into products of commercial interest via a phosphorylated route, production of Lycopene from Gluconate was attempted. For such a purpose, a synthetic operon containing the crtE, crtI and crtB genes from Erwinia uredovora (Misawa et al., 1990) was synthesized by GenScript (Piscataway, NJ) (SEQ ID NO: 79). To express these genes in E.coli, Primers SEQ ID NO. 80 and SEQ ID NO: 81 were used to amplify the whole operon by PCR. The PCR product was digested with restriction enzymes NcoI and HindIII and cloned into the expression vector pBAD/Myc-His A (Invitrogen, Carlsbad, CA) digested with the same enzymes. After transformation and plating on LA agar /Carbenicillin 100 µg/ml plates, three colonies exhibiting a reddish color were purified, and analyzed by restriction analysis using enzymes EcoRV and BamHI. All three colonies produced the correct size DNA fragments. DNA from two 2 colonies were combined and named pBAD-crtEIB24.

Synthetic operon containing the crtE, crtI and crtB genes from Erwinia uredovora

ATGACGGTCTGCGCAAAAAAACACGTTCATCTCACTCGCGATGCTGCGGA
GCAGTTACTGGCTGATATTGATCGACGCCTTGATCAGTTATTGCCCGTGG
AGGGAGAACGGGATGTTGTGGGTGCCGCGATGCGTGAAGGTGCGCTGGCA
CCGGGAAAACGTATTCGCCCCATGTTGCTGTTGCTGACCGCCCGCGATCT
GGGTTGCGCTGTCAGCCATGACGGATTACTGGATTTGGCCTGTGCGGTGG
AAATGGTCCACGCGGCTTCGCTGATCCTTGACGATATGCCCTGCATGGAC
GATGCGAAGCTGCGGCGCGGACGCCCTACCATTCATTCTCATTACGGAGA
GCATGTGGCAATACTGGCGGCGGTTGCCTTGCTGAGTAAAGCCTTTGGCG
TAATTGCCGATGCAGATGGCCTCACGCCGCTGGCAAAAAATCGGGCGGTT
TCTGAACTGTCAAACGCCATCGGCATGCAAGGATTGGTTCAGGGTCAGTT
CAAGGATCTGTCTGAAGGGGATAAGCCGCGCAGCGCTGAAGCTATTTTGA
TGACGAATCACTTTAAAACCAGCACGCTGTTTTGTGCCTCCATGCAGATG
GCCTCGATTGTTGCGAATGCCTCCAGCGAAGCGCGTGATTGCCTGCATCG
TTTTTCACTTGATCTTGGTCAGGCATTTCAACTGCTGGACGATTTGACCG
ATGGCATGACCGACACCGGTAAGGATAGCAATCAGGACGCCGGTAAATCG
ACGCTGGTCAATCTGTTAGGCCCGAGGGCGGTTGAAGAACGTCTGAGACA
ACATCTTCAGCTTGCCAGTGAGCATCTCTCTGCGGCCTGCCAACACGGGC
ACGCCACTCAACATTTTATTCAGGCCTGGTTTGACAAAAAACTCGCTGCC
GTCAGTTAATAAGCTAAGGAAAAACAAAATGAAACCAACTACGGTAATTG
GTGCAGGCTTCGGTGGCCTGGCACTGGCAATTCGTCTACAAGCTGCGGGG
ATCCCCGTCTTACTGCTTGAACAACGTGATAAACCCGGCGGTCGGGCTTA
TGTCTACGAGGATCAGGGGTTTACCTTTGATGCAGGCCCGACGGTTATCA
CCGATCCCAGTGCCATTGAAGAACTGTTTGCACTGGCAGGAAAACAGTTA
AAAGAGTATGTCGAACTGCTGCCGGTTACGCCGTTTTACCGCCTGTGTTG
GGAGTCAGGGAAGGTCTTTAATTACGATAACGATCAAACCCGGCTCGAAG
CGCAGATTCAGCAGTTTAATCCCCGCGATGTCGAAGGTTATCGTCAGTTT
CTGGACTATTCACGCGCGGTGTTTAAAGAAGGCTATCTAAAGCTCGGTAC
TGTCCCTTTTTTATCGTTCAGAGACATGCTTCGCGCCGCACCTCAACTGG
CGAAACTGCAGGCATGGAGAAGCGTTTACAGTAAGGTTGCCAGTTACATC
GAAGATGAACATCTGCGCCAGGCGTTTTCTTTCCACTCGCTGTTGGTGGG
CGGCAATCCCTTCGCCACCTCATCCATTTATACGTTGATACACGCGCTGG
AGCGTGAGTGGGGCGTCTGGTTTCCGCGTGGCGGCACCGGCGCATTAGTT
CAGGGGATGATAAAGCTGTTTCAGGATCTGGGTGGCGAAGTCGTGTTAAA
CGCCAGAGTCAGCCATATGGAAACGACAGGAAACAAGATTGAAGCCGTGC
ATTTAGAGGACGGTCGCAGGTTCCTGACGCAAGCCGTCGCGTCAAATGCA
GATGTGGTTCATACCTATCGCGACCTGTTAAGCCAGCACCCTGCCGCGGT
TAAGCAGTCCAACAAACTGCAGACTAAGCGCATGAGTAACTCTCTGTTTG
TGCTCTATTTTGGTTTGAATCACCATCATGATCAGCTCGCGCATCACACG
GTTTGTTTCGGCCCGCGTTACCGCGAGCTGATTGACGAAATTTTTAATCA
TGATGGCCTCGCAGAGGACTTCTCACTTTATCTGCACGCGCCCTGTGTCA
CGGATTCGTCACTGGCGCCTGAAGGTTGCGGCAGTTACTATGTGTTGGCG
CCGGTGCCGCATTTAGGCACCGCGAACCTCGACTGGACGGTTGAGGGGCC
AAAACTACGCGACCGTATTTTTGCGTACCTTGAGCAGCATTACATGCCTG
GCTTACGGAGTCAGCTGGTCACGCACCGGATGTTTACGCCGTTTGATTTT
CGCGACCAGCTTAATGCCTATCATGGCTCAGCCTTTTCTGTGGAGCCCGT
TCTTACCCAGAGCGCCTGGTTTCGGCCGCATAACCGCGATAAAACCATTA
CTAATCTCTACCTGGTCGGCGCAGGCACGCATCCCGGCGCAGGCATTCCT
GGCGTCATCGGCTCGGCAAAAGCGACAGCAGGTTTGATGCTGGAGGATCT
GATATGAATAATCCGTCGTTACTCAATCATGCGGTCGAAACGATGGCAGT
TGGCTCGAAAAGTTTTGCGACAGCCTCAAAGTTATTTGATGCAAAAACCC
GGCGCAGCGTACTGATGCTCTACGCCTGGTGCCGCCATTGTGACGATGTT
ATTGACGATCAGACGCTGGGCTTTCAGGCCCGGCAGCCTGCCTTACAAAC
GCCCGAACAACGTCTGATGCAACTTGAGATGAAAACGCGCCAGGCCTATG
CAGGATCGCAGATGCACGAACCGGCGTTTGCGGCTTTTCAGGAAGTGGCT
ATGGCTCATGATATCGCCCCGGCTTACGCGTTTGATCATCTGGAAGGCTT
CGCGATGGATGTACGCGAAGCGCAATACAGCCAACTGGATGATACGCTGC
GCTATTGCTATCACGTTGCAGGCGTTGTCGGCTTGATGATGGCGCAAATC
ATGGGCGTGCGGGATAACGCCACGCTGGACCGCGCCTGTGACCTTGGGCT
GGCATTTCAGTTGACCAATATTGCTCGCGATATTGTGGACGATGCGCATG
CGGGCCGCTGTTATCTGCCGGCAAGCTGGCTGGAGCATGAAGGTCTGAAC
AAAGAGAATTATGCGGCACCTGAAAACCGTCAGGCGCTGAGCCGTATCGC
CCGTCGTTTGGTGCAGGAAGCAGAACCTTACTATTTGTCTGCCACAGCCG
GCCTGGCAGGGTTGCCCCTGCGTTCCGCCTGGGCAATCGCTACGGCGAAG
CAGGTTTACCGGAAAATAGGTGTCAAAGTTGAACAGGCCGGTCAGCAAGC
CTGGGATCAGCGGCAGTCAACGACCACGCCCGAAAAATTAACGCTGCTGC
TGGCCGCCTCTGGTCAGGCCCTTACTTCCCGGATGCGGGCTCATCCTCCC
CGCCCTGCGCATCTCTGGCAGCGCCCGCTCTAATAAGCTT (SEQ ID N
O: 79)

### Example17 Lycopene Production Assay

Production of lycopene was measured essentially as described previously (Min-Jung et al., 2005). Briefly, after measuring OD₆₀₀ of each culture, cells from 1 ml were harvested by centrifugation at 20,000 ^{x}g for 5 min, washed once with water and pelleted again at 20,000 ^{x}g for 5 min. Cells were resuspended with 800 µL of acetone and incubated at 55° C. for 15 min in the dark. Samples were centrifuged at 20,000 ^{x}g for 5 min. 650 µL of the acetone supernatant were transferred to clean tube. Lycopene absorption was measured at 475 nm, and values were normalized by OD₆₀₀.

### Example 18 Production of Lycopene From Gluconate in W3110 Strain

To test Lycopene production in W3110 wild type strain, plasmids pBP2 and pAchro-L1 (described in Example 12 above) were transformed into electrocompetent cells of strain W3110. One colony containing pBP2 or pAchro-L1 were purified and transformed with plasmid pBAD-crtEIB24 (described in Example 16 above). Transformants were selected on LA Carbenicillin 100 µg/ml and Spectinomycin 100 µg/ml plates. After 24 hr incubation at 37° C., all colonies had a reddish color. Two different colonies from each plasmid combination were purified and used to prepare frozen glycerol stocks. For lycopene production evaluation, 2.5 ml of Lb media containing 200 µg/ml of carbenicillin and 200 µg/ml spectinomycin were inoculated directly from frozen vials. After 24 hr incubation at 30° C. at 200 rpm, 25 µL of the Lb cultures were transferred to 15 ml tubes with 2.5 ml of Hi-Def Azure media (Teknova Inc. Hollister, CA) containing 2% Glucuronate, 200 µg/ml of carbenicillin and spectinomycin 200 µg/ml.

After 24 hr incubation at 37° C. at 200 rpm, 25 µL of the Glucuronate cultures were transferred to 15 ml tubes with 2.5 ml of Hi-Def Azure media containing 2% Gluconate, 200 µg/ml of carbenicillin and 200 µg/ml spectinomycin. Gluconate cultures were incubated for 24 hr at 3° C. at 200 rpm. After OD₆₀₀ was measured, 1 ml sample were taken for lycopene analysis as described in Example 17.

Results are shown in Table 6. As can be seen all the strains reached the same final OD and produced the same amount of lycopene. This indicates that the presence of Gluconate dehydratase doesn’t increase lycopene production. In wild type W3110 strain after gluconate transport inside of the cells, gluconate is first converted into Gluconate-6-phosphate, which then is metabolized by the Pentoses and the Entner-Doudoroff (ED) pathways. The amount of gluconate metabolized by each one these two pathways is unknown. However, only the ED pathway produces directly Pyruvate and Glyceraldehyde-3-phosphate (Peekhaus and Conway, 1998). The fact that the addition of Gluconate dehydratase did not have any effect on growth or lycopene production suggest that the activities of the Pentose and ED pathways outcompete Gluconate dehydratase activity.

TABLE 6

| Strain / plasmids | OD₆₀₀ (⅒ dilution) | OD₄₇₅ | OD₄₇₅/OD₆₀₀ |
|---|---|---|---|
| W3110 / pBP2, pBADcrtEIB24 col 1 | 0.33 | 0.389 | 1.19 |
| W3110 / pBP2, pBADcrtEIB24 col 2 | 0.31 | 0.378 | 1.22 |
| W3110 /pAchro-L1, pBADcrtEIB24 col 1 | 0.33 | 0.386 | 1.16 |
| W3110 / pAchro-L1, pBADcrtEIB24 col 2 | 0.33 | 0.426 | 1.29 |

### Example 19 Production of Lycopene From Gluconate in Strain Δ3-Km Strain

Results obtained in example 18 indicate that in a wild type background, the enzyme Gluconate dehydratase was unable to outcompete the enzymes that produce Gluconate-6-phosphate, which is the entry metabolite for the pentose and the ED pathways. To show that elimination of these competing pathways favor the conversion of gluconate into KDG and lycopene, strain Δ3-Km described in Example 3 above, was transformed with pBP2, pAchro-L1 and pBADcrtEIB24. All the procedures described in Example 20 above for strain construction, lycopene production and evaluation were repeated. Results from this experiment are shown in Table 7. As can be seen, and as shown in Example 11 above, strain Δ3-Km without Gluconate dehydratase had a limited growth. However, the addition of Gluconate dehydratase caused a 2.7-fold increase in OD₆₀₀, indicating that gluconate was metabolized via KDG. After correcting for ODs differences (see Table 7), strains carrying Gluconate dehydratase produced 3-fold more lycopene than the strains carrying the empty vector pBP2.

Lycopene production could be further increased if less KDG is used for growth, which is accomplished by further generic modifications and/or by changing culture conditions.

TABLE 7

| Strain / plasmids | OD₆₀₀ (⅒ dilution) | OD₄₇₅ | OD₄₇₅/OD₆₀₀ |
|---|---|---|---|
| Δ3-Km / pBP2, pBADcrtEIB24 col 1 | 0.16 | 0.074 | 0.46 |
| Δ3-Km / pBP2, pBADcrtEIB24 col 2 | 0.14 | 0.089 | 0.64 |
| Δ3-Km / pAchro-L1, pBADcrtEIB24 col 1 | 0.42 | 0.684 | 1.63 |
| Δ3-Km / pAchro-L1, pBADcrtEIB24 col 2 | 0.40 | 0.665 | 1.66 |

These non-limiting results confirm that expression of exogenous gluconate dehydratase enables the described strains to assimilate gluconate via a KDG intermediate, which in turn, can be utilized to produce more efficiently, products of commercial interest.

While illustrative embodiments have been illustrated and described herein, it will be appreciated that various changes can be made therein without departing from the spirit and scope of the contents described herein.

### REFERENCES

Baba T. et al., (2006). Construction of Escherichia coli K-12 in-frame, single-gene knockout mutants: the Keio collection. Mol. Syst. Biol. 2:1-11.

Datsenko K.A., and Wanner B.L.(2000). One-step inactivation of chromosomal genes in Escherichia coli K-12 using PCR products. PNAS. 97: 6640-6645.

Guterl J. et al., (2012). Cell-free metabolic engineering: Production of chemicals by minimized reaction cascades. Chem. Sus. Chem. 5: 2165-2172.

Kim & Lee., (2008). Identification and Characterization of the bacterial D-Gluconate dehydratase in Achromobacter xylosoxidans . Biotech. Bioproc. Eng. 13: 436-444.

Lerner C.L. and Inouye M. (1990). Low copy number plasmids for regulated low-level expression of cloned genes in Escherichia coli with blue/white insert screening capability. 18(15): 4631.

Li C. et al., (2015). Modification of targets related to the EntnerDoudoroff/pentose phosphate pathway route for methyl-D-erythritol-4-phosphate-dependent carotenoid biosynthesis in Escherichia coli. Microb. Cell Fact. 14:117.

Matsubara K. et al., (2014). One-step synthesis of 2-keto-3-deoxy-D-gluconate by biocatalytic dehydration of D-gluconate. J. Biotechnol. 69-77.

Min-Jung K. et al., (2005). Enhancement of Lycopene Production in Escherichia coli by Optimization of the Lycopene Synthetic Pathway. J. Mol. Biotechnol. 15: 880-886.

Misawa N. et al., (1990). Elucidation of the Erwinia uredovora Carotenoid Biosynthetic Pathway by Functional Analysis of Gene Products Expressed in Escherichia coli. J. Bact. 172: 6704-6712.

Morita K. et al., (2017). Heterologous expression of bacterial phosphoenol pyruvate carboxylase and Entner-Doudoroff pathway in Saccharomyces cerevisiae for improvement of isobutanol production. J. Biosci. Bioeng.124:263-270.

Nagai H. et al., (2018). Metabolic engineering of mevalonate-producing Escherichia coli strains based on thermodynamic analysis. Met. Eng. 47: 1-9.

Peekhaus N. and Conway T. (1998). What is for Dinner?: Entner-Doudoroff Metabolism in Escherichia coli. J. Bacteriol. 180:3495-3502

Taniguchi H. et al., (2017). Modules for in vitro metabolic engineering: Pathway assembly for bio-based production of value-added chemicals. Synth. Syst. Biotech. 2: 65-74.

Tong S., et al. (1996). Cloning and molecular genetic characterization of the Escherichia coli gntR, gntK, and gntU genes of GntI, the main system for gluconate metabolism. J. Bact. 178: 3260-3269.

Yang M. et al., (2017) . Switch on a more efficient pyruvate synthesis pathway based on transcriptome analysis and metabolic evolution. J. Biosci. Bioeng.124: 523-527.

Yang J. Guo L. (2014). Biosynthesis of 13-carotene in engineered E. coli using the MEP and MVA pathways. Microb. Cell Factories. 13: 160.

1. A recombinant cell comprising a gene encoding a heterologous gluconate dehydratase, wherein the recombinant cell further comprises one or more genetic modifications resulting in at least one, any two, any three, any four, or all five of the following phenotypes:
(a) decrease or elimination of glucose assimilation;
(b) decrease or elimination of gluconate-6-phosphate production from gluconate;
(c) decrease or elimination of glucose-6-phosphate production from glucose;
(d) decrease or elimination of 2-keto-3-deoxy-D-gluconate (KDG) phosphorylation; and
(e) decrease or elimination of KDG degradation by non-phosphorylative cellular reactions that consume KDG.

2. A recombinant cell comprising a genetic modification resulting in the decrease or elimination of 2-keto-3-deoxy-D-gluconate phosphorylation, wherein the recombinant cell further comprises one or more genetic modifications resulting in at least one, any two, any three, or all four of the following phenotypes:
(a) decrease or elimination of glucose assimilation;
(b) decrease or elimination of gluconate-6-phosphate production from gluconate; and
(c) decrease or elimination of glucose-6-phosphate production from glucose; and
(d) decrease or elimination of KDG degradation by non-phosphorylative cellular reactions that consume KDG.

3-53. (canceled)

54. A method of producing 2-keto-3-deoxy gluconate (KDG) comprising the steps of:
(a) culturing a recombinant cell in a suitable culture medium, wherein the recombinant cell comprises a gene encoding a heterologous gluconate dehydratase enzyme having at least 70% sequence identity to SEQ ID NO: 1; and
(b) allowing expression of said gene, wherein said expression results in the production of KDG;
wherein said production of KDG is performed at a temperature between 20° C. and 45° C.

55. The method according to claim 54, wherein the gene encoding a heterologous gluconate dehydratase comprises a nucleic acid sequence having at least 70% sequence identity to SEQ ID NO: 2 or 3.

56. The method according to claim 54, wherein the gene encoding a heterologous gluconate dehydratase comprises a nucleic acid sequence having at least 70% sequence identity to SEQ ID NO: 4.

57. The method according to claim 54, wherein the culture medium comprises glycerol and glucose.

58. The method according to claim 54, wherein the yield of KDG is at least 60%.

59-60. (canceled)

61. The method according to claim 54, the method further comprising purifying KDG from cell culture.

62. The method according to claim 54, the method further comprising purifying KDG from supernatant.

63-64. (canceled)

65. The method according to claim 54, the method further comprising converting KDG to a 2′-deoxynucleoside or a precursor thereof.

66. The method according to claim 54, the method further comprising converting KDG to 5-hydroxymethyl-2-furoic acid (HMFA) and/or furan dicarboxylic acid (FDCA).

67. The method according to claim 54, wherein the recombinant cell is defined according to claim 1.

68. The method according to claim 54, wherein the recombinant cell further comprises genetic modifications resulting in one or more of the following phenotypes:
(a) increased production of pyruvate and/or glyceraldehyde-3-phosphate from KDG;
(b) increased production of isopentenyl pyrophosphate (IPP) and/or dimethylallyl pyrophosphate (DMAPP) from KDG; and
(c) increased production of terpenoids from KDG.

69. The method according to claim 68, wherein the genetic modifications resulting in increased production of:
(a) pyruvate and/or glyceralehyde-3-phosphate from KDG comprises increasing expression of a KDG/KDPG aldolase;
(b) pyruvate and/or glyceralehyde-3-phosphate from KDG comprises increasing expression of a KDG kinase;
(c) IPP or DMAPP from KDG comprises increasing expression of enzymes involved in the non-mevalonate (MEP) pathway; and
(d) terpenoids from KDG comprises increasing expression of enzymes involved in the terpenoid biosynthetic pathway.

70. The method according to claim 68, the method further comprising a step of purifying from cell culture:
(a) pyruvate and/or glyceraldehyde-3-phosphate;
(b) IPP or DMAPP; and/or
(c) terpenoids.

71-93. (canceled)

94. A nucleic acid comprising a nucleic acid sequence having at least 70% sequence identity to SEQ ID NO: 2. or SEQ ID NO: 3 or SEQ ID NO: 4, wherein the nucleic acid is isolated, recombinant or synthetic.

95. (canceled)

96. A vector comprising the nucleic acid according to claim 94.

97-103. (canceled)

104. A recombinant cell comprising a vector according to claim 96.

105. The method of claim 70, wherein the terpenoids comprise one or more isoprene units.


SEQUENCE LISTING (81 sequences)

SEQ ID NO: 1 - protein, 593 aa, Artificial Sequence
  Synthetic, isolated, putified or recombinnant gluconate dehydratase
MSQTPRKLRSQKWFDDPAHADMTAIYVERYLNYGLTRQELQSGRPIIGIAQTGSDLAPCNRHHLALAERIKAGIRDAGGIPMEFPVHPLAEQGRRPTAALDRNLAYLGLVEILHGYPLDGVVLTTGCDKTTPACLMAAATVDIPAIVLSGGPMLDGWHDGQRVGSGTVIWHARNLMAAGKLDYEGFMTLATASSPSVGHCNTMGTALSMNSLAEALGMSLPTCASIPAPYRERGQMAYATGLRICDMVREDLRPSHILTRQAFENAIVVASALGASSNCPPHLIAMARHAGIDLSLDDWQRLGEDVPLLVNCVPAGEHLGEGFHRAGGVPAVLHELAAAGRLHTDCGTVSGKTIGEIAATAKTNNADVIRSCDAPLRHRAGFIVLSGNFFDSAIIKMSVVGEAFRRAYLSEPGSENAFEARAIVFEGPEDYHARIEDPSLNIDEHCILVIRGAGTVGYPGSAEVVNMAPPSHLLKRGIDSLPCLGDGRQSGTSASPSILNMSPEAAVGGGLALLRTGDRIRVDLNQRSVTALVDETEMERRKLEPPYQAPESQTPWQELYRQLVGQLSTGGCLEPATLYLKVVETRGDPRHSH

SEQ ID NO: 2 - DNA, 1782 bp, Achromobacter sp. K91
atgtcccagactccccgcaagctgcgcagccagaagtggttcgacgatccggcccacgccgacatgacggccatctatgtcgagcgctacctcaactacggcctgacgcgccaggaactgcaatctgggcggccgatcatcggcatcgctcagaccggcagcgacctggcgccctgcaaccggcatcacctggcgctggccgagcgcatcaaggccggcatccgcgacgcgggcggcatccccatggagtttcccgtgcacccgctggccgaacagggccggcgccccaccgccgcgctggaccgcaacctggcctatctgggcctggtagagatcctgcacggctatccgctggacggcgtggtcctgaccaccggctgcgacaagaccacgcccgcctgcctcatggccgcggccaccgtggatattcccgccatcgtgctgtccggcgggccgatgctggacggctggcacgacggccagcgcgtcggctccggcacggtcatctggcatgcgcgcaatctgatggccgcgggcaagctggactacgaaggcttcatgacgctggccacggcgtcgtcgccgtccgtcggccactgcaacaccatgggcacggcgctgtcgatgaactcgctggccgaagcgctgggcatgtcgctgcccacctgcgccagcattcccgcgccgtaccgcgagcgcggccagatggcctacgccaccgggctgcgtatctgcgacatggtgcgcgaggacttgcgcccgtcccacatcctgacgcggcaggctttcgagaacgccatcgtcgtggcctcggcgctgggcgcctccagcaactgcccgccgcacctcatcgccatggcgcgccacgccggcatcgacctgagcctggacgactggcagcgcttgggcgaagacgtgccgctgctggtcaactgtgtacctgcgggcgagcatctgggcgaaggctttcaccgcgcgggcggcgtcccggcggtgttgcacgaactggcggcagcaggccgcctgcataccgattgcggcaccgtgtccggcaagaccatcggcgagatcgcggctacggccaagaccaacaatgcggacgtcatccgcagctgcgacgcgccgctcaggcatcgcgcgggattcatcgtcctctcgggcaacttcttcgacagcgccatcatcaagatgtcggtcgtcggcgaagccttccgccgcgcctatctgtcggagcccggttcagagaacgccttcgaggcgcgcgccatcgtcttcgaaggccccgaggactaccacgcgcgcatcgaagacccctccctgaacatcgacgagcactgcatcctggtgatccgcggcgccggcaccgtcggctaccccggcagcgcggaagtggtcaacatggcgccgccctcgcatctgctcaagcgcggcatagactctctgccctgcctgggcgacggccgccagagcggcacctcggccagcccttcgatcctgaacatgtcgccggaagccgcggtgggcggtggcctcgcgctgttgcgcacgggcgaccgcatccgcgtcgacctgaatcagcgctcggtcaccgccctggtggatgaaacagaaatggaacgccgcaagttggagccgccctaccaggcgccggaatctcagacgccgtggcaagagctgtaccggcaactggtcggccagctttcaaccggcggctgcctggagcccgccaccttgtatctgaaggtcgtggaaacacggggcgatccccggcattcgcattga

SEQ ID NO: 3 - DNA, 1782 bp, Achromobacter sp. 2789STDY5608625
atgtcccagactccccgcaagctgcgcagccagaagtggttcgacgatccggcccacgccgacatgacggccatctatgtcgagcgctacctcaactacggcctgacgcgccaggaactgcaatctgggcggccgatcatcggcatcgctcagaccggcagcgacctggcgccctgcaaccggcatcacctggcgctggccgagcgcatcaaggccggcatccgcgacgcgggcggcatccccatggagtttcccgtgcacccgctggccgaacagggccggcgccccaccgccgcgctggaccgcaacctggcctatctgggcctggtagagatcctgcacggctatccgctggacggcgtggtcctgaccaccggctgcgacaagaccacgcccgcctgcctcatggccgcggccaccgtggatattcccgccatcgtgctgtccggcgggccgatgctggacggctggcacgacggccagcgcgtcggctccggcacggtcatctggcatgcgcgcaatctgatggccgcgggcaagctggactacgaaggcttcatgacgctggccacggcgtcgtcgccgtccgtcggccactgcaacaccatgggcacggcgctgtcgatgaactcgctggccgaagcgctgggcatgtcgctgcccacctgcgccagcattcccgcgccgtaccgcgagcgcggccagatggcctacgccaccgggctgcgtatctgcgacatggtgcgcgaggacttgcgcccatcccatatcctgacgcggcaggctttcgagaacgccatcgtcgtggcctcggcgctgggcgcctccagcaactgcccgccgcacctcatcgccatggcccgccacgccggcatcgacctgagcctggacgactggcagcgcttgggcgaagacgtgccgctgctggtcaactgtgtacctgcgggtgagcatctgggcgaaggctttcaccgcgcgggcggcgtcccggcggtgttgcacgaactggcggcagcaggccgcctgcataccgattgcggcaccgtgtccggcaagaccatcggcgagatcgcggctacggccaagaccaacaatgcggacgtcatccgcagctgcgacgcgccgctcaggcatcgcgcgggattcatcgtcctctcgggcaacttcttcgacagcgccatcatcaagatgtcggtcgtcggcgaagccttccgccgcgcctatctgtcggagcccggttcagagaacgccttcgaggcgcgcgccatcgtcttcgaaggccccgaggactaccacgcgcgcatcgaagacccctccctgaacatcgacgagcactgcatcctggtgatccgcggcgccggcaccgtcggctaccccggcagcgcggaagtggtcaacatggcgccgccctcgcatctgctcaagcgcggcatagactctctgccctgcctgggcgacggccgccagagcggcacctcggccagcccttcgatcctgaacatgtcgccggaagccgcggtgggcggtggcctcgcgctgttgcgcacgggcgaccgcatccgcgtcgacctgaaccagcgctcggtcaccgccctggtggatgaaacagaaatggaacgccgcaagctggagccgccctaccaggcgccggaatctcagacgccgtggcaagagctataccggcaactggtcggccagctttcaaccggcggctgcctggagcccgccaccttgtatctgaaggtcgtggaaacgcggggcgatccccggcattcgcattga

SEQ ID NO: 4 - DNA, 1782 bp, Artificial Sequence
  Nucleic acid sequence encoding the gluconate dehydratase polypeptide of SEQ ID NO: 1, which has been codon optimized for expression in E. coli
atgtcgcaaactcctcgcaaactgcgttcgcagaagtggtttgacgatccggcacatgcagatatgacggccatctatgtcgaacgttacttgaactacgggttgacccgtcaagagctgcaaagtggccgcccgatcattggaattgcccaaactgggtccgatttagctccttgtaaccgccatcatcttgcacttgctgagcgtatcaaggccggaattcgtgacgccggtggcatcccgatggaatttcctgtgcatcccttggcggaacagggccgtcgtccaactgcggccttagaccgcaacttagcatatcttgggctggtcgaaatcctgcacgggtatcccttggacggagttgtgctgacgacaggttgcgacaagaccacccctgcttgtctgatggcagctgcaacagtggacatcccggcgattgtgctgagcggcggacccatgcttgacgggtggcacgacggccagcgcgtgggcagtggaacagtgatctggcacgcgcgtaatttaatggcagcgggtaaattagattacgaaggttttatgacactggccactgctagcagtccctccgtcggtcattgcaacacaatggggacggcacttagcatgaactcgctggcagaagctctgggaatgtccttaccgacatgcgcatcaatcccggccccctatcgcgaacgtgggcaaatggcatacgcgactggtttgcgcatctgcgacatggtacgtgaggatcttcgtccctcgcacatccttactcgtcaggcctttgaaaacgcgattgtcgttgcctcagctttgggcgcgagtagtaactgcccacctcatttgattgcgatggcccgtcacgccggaatcgatttatcgcttgatgattggcaacgccttggagaagatgttccgttgcttgtcaattgcgttcccgcgggtgagcatcttggggagggattccaccgtgccgggggcgtacctgccgttcttcacgaattggcagcggctgggcgccttcacaccgactgcggaaccgtttccgggaaaacgatcggcgagatcgcagcaacagccaagactaataacgcagatgtaatccgttcttgtgatgctccccttcgtcaccgcgccgggttcatcgtattatcagggaacttttttgactccgccatcatcaaaatgtcggtcgtaggcgaggcattccgccgcgcatacttgtcggaaccgggttcagaaaatgcgttcgaagctcgtgcgattgtgtttgagggcccagaagactaccatgcgcgcattgaagatccttctcttaatattgacgaacattgtatcctggtaatccgcggagcggggacggtgggttacccaggttcggcggaagtagtcaatatggctcctcccagtcaccttttaaaacgcggtattgactcattaccgtgtttaggggatggtcgccagagcggaacgagtgcatctcccagcatcttgaatatgtctccagaggccgcggtcgggggaggcttggcgcttctgcgcaccggtgaccgcattcgtgttgacctgaaccagcgctcagttacggcgcttgtcgatgagactgaaatggagcgtcgtaaattggaaccaccttatcaggccccagaatcgcagaccccgtggcaagagttgtatcgccagttagttgggcaattgtccacgggaggatgtcttgagcccgcgacattgtaccttaaggtcgtagagacacgtggtgatccgcgtcactctcactaa

SEQ ID NO: 5 - DNA, 4356 bp, Artificial Sequence
  Vector pTrc-His2C
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccgctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 6 - DNA, 6138 bp, Artificial Sequence
  Vector pAchro-10
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtcgcaaactcctcgcaaactgcgttcgcagaagtggtttgacgatccggcacatgcagatatgacggccatctatgtcgaacgttacttgaactacgggttgacccgtcaagagctgcaaagtggccgcccgatcattggaattgcccaaactgggtccgatttagctccttgtaaccgccatcatcttgcacttgctgagcgtatcaaggccggaattcgtgacgccggtggcatcccgatggaatttcctgtgcatcccttggcggaacagggccgtcgtccaactgcggccttagaccgcaacttagcatatcttgggctggtcgaaatcctgcacgggtatcccttggacggagttgtgctgacgacaggttgcgacaagaccacccctgcttgtctgatggcagctgcaacagtggacatcccggcgattgtgctgagcggcggacccatgcttgacgggtggcacgacggccagcgcgtgggcagtggaacagtgatctggcacgcgcgtaatttaatggcagcgggtaaattagattacgaaggttttatgacactggccactgctagcagtccctccgtcggtcattgcaacacaatggggacggcacttagcatgaactcgctggcagaagctctgggaatgtccttaccgacatgcgcatcaatcccggccccctatcgcgaacgtgggcaaatggcatacgcgactggtttgcgcatctgcgacatggtacgtgaggatcttcgtccctcgcacatccttactcgtcaggcctttgaaaacgcgattgtcgttgcctcagctttgggcgcgagtagtaactgcccacctcatttgattgcgatggcccgtcacgccggaatcgatttatcgcttgatgattggcaacgccttggagaagatgttccgttgcttgtcaattgcgttcccgcgggtgagcatcttggggagggattccaccgtgccgggggcgtacctgccgttcttcacgaattggcagcggctgggcgccttcacaccgactgcggaaccgtttccgggaaaacgatcggcgagatcgcagcaacagccaagactaataacgcagatgtaatccgttcttgtgatgctccccttcgtcaccgcgccgggttcatcgtattatcagggaacttttttgactccgccatcatcaaaatgtcggtcgtaggcgaggcattccgccgcgcatacttgtcggaaccgggttcagaaaatgcgttcgaagctcgtgcgattgtgtttgagggcccagaagactaccatgcgcgcattgaagatccttctcttaatattgacgaacattgtatcctggtaatccgcggagcggggacggtgggttacccaggttcggcggaagtagtcaatatggctcctcccagtcaccttttaaaacgcggtattgactcattaccgtgtttaggggatggtcgccagagcggaacgagtgcatctcccagcatcttgaatatgtctccagaggccgcggtcgggggaggcttggcgcttctgcgcaccggtgaccgcattcgtgttgacctgaaccagcgctcagttacggcgcttgtcgatgagactgaaatggagcgtcgtaaattggaaccaccttatcaggccccagaatcgcagaccccgtggcaagagttgtatcgccagttagttgggcaattgtccacgggaggatgtcttgagcccgcgacattgtaccttaaggtcgtagagacacgtggtgatccgcgtcactctcactaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 7 - DNA, 5770 bp, Artificial Sequence
  Vector pCL-BP2
cgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccgctttctagaacaaaaactcatctcagaagaggatctgaatagcgccgtcgaccatcatcatcatcatcattgagtttaaaccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgt

SEQ ID NO: 8 - DNA, 7552 bp, Artificial Sequence
  Vector pAchro-L1
cgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtcgcaaactcctcgcaaactgcgttcgcagaagtggtttgacgatccggcacatgcagatatgacggccatctatgtcgaacgttacttgaactacgggttgacccgtcaagagctgcaaagtggccgcccgatcattggaattgcccaaactgggtccgatttagctccttgtaaccgccatcatcttgcacttgctgagcgtatcaaggccggaattcgtgacgccggtggcatcccgatggaatttcctgtgcatcccttggcggaacagggccgtcgtccaactgcggccttagaccgcaacttagcatatcttgggctggtcgaaatcctgcacgggtatcccttggacggagttgtgctgacgacaggttgcgacaagaccacccctgcttgtctgatggcagctgcaacagtggacatcccggcgattgtgctgagcggcggacccatgcttgacgggtggcacgacggccagcgcgtgggcagtggaacagtgatctggcacgcgcgtaatttaatggcagcgggtaaattagattacgaaggttttatgacactggccactgctagcagtccctccgtcggtcattgcaacacaatggggacggcacttagcatgaactcgctggcagaagctctgggaatgtccttaccgacatgcgcatcaatcccggccccctatcgcgaacgtgggcaaatggcatacgcgactggtttgcgcatctgcgacatggtacgtgaggatcttcgtccctcgcacatccttactcgtcaggcctttgaaaacgcgattgtcgttgcctcagctttgggcgcgagtagtaactgcccacctcatttgattgcgatggcccgtcacgccggaatcgatttatcgcttgatgattggcaacgccttggagaagatgttccgttgcttgtcaattgcgttcccgcgggtgagcatcttggggagggattccaccgtgccgggggcgtacctgccgttcttcacgaattggcagcggctgggcgccttcacaccgactgcggaaccgtttccgggaaaacgatcggcgagatcgcagcaacagccaagactaataacgcagatgtaatccgttcttgtgatgctccccttcgtcaccgcgccgggttcatcgtattatcagggaacttttttgactccgccatcatcaaaatgtcggtcgtaggcgaggcattccgccgcgcatacttgtcggaaccgggttcagaaaatgcgttcgaagctcgtgcgattgtgtttgagggcccagaagactaccatgcgcgcattgaagatccttctcttaatattgacgaacattgtatcctggtaatccgcggagcggggacggtgggttacccaggttcggcggaagtagtcaatatggctcctcccagtcaccttttaaaacgcggtattgactcattaccgtgtttaggggatggtcgccagagcggaacgagtgcatctcccagcatcttgaatatgtctccagaggccgcggtcgggggaggcttggcgcttctgcgcaccggtgaccgcattcgtgttgacctgaaccagcgctcagttacggcgcttgtcgatgagactgaaatggagcgtcgtaaattggaaccaccttatcaggccccagaatcgcagaccccgtggcaagagttgtatcgccagttagttgggcaattgtccacgggaggatgtcttgagcccgcgacattgtaccttaaggtcgtagagacacgtggtgatccgcgtcactctcactaagctttctagaacaaaaactcatctcagaagaggatctgaatagcgccgtcgaccatcatcatcatcatcattgagtttaaaccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgt

SEQ ID NO: 9 - DNA, 50 bp, Artificial Sequence
  Synthetic oligonucleotide
ctagactttagttccacaacactaaacctataagttggggaaatacaatg

SEQ ID NO: 10 - DNA, 50 bp, Artificial Sequence
  Synthetic oligonucleotide
atgggcgccatttttcactgcggcaagaattacttcttgatgcggataac

SEQ ID NO: 11 - DNA, 27 bp, Artificial Sequence
  Synthetic oligonucleotide
ccccaacttataggtttagtgttgtgg

SEQ ID NO: 12 - DNA, 21 bp, Artificial Sequence
  Synthetic oligonucleotide
cctggtgctgacggaaggtgc

SEQ ID NO: 13 - DNA, 34 bp, Artificial Sequence
  Synthetic oligonucleotide
ggttatccgcatcaagaagtaattcttgccgcag

SEQ ID NO: 14 - DNA, 22 bp, Artificial Sequence
  Synthetic oligonucleotide
ccctccagcatggcgttgatgc

SEQ ID NO: 15 - DNA, 25 bp, Artificial Sequence
  Synthetic oligonucleotide
cgcccctcctggcattgattcagcc

SEQ ID NO: 16 - DNA, 22 bp, Artificial Sequence
  Synthetic oligonucleotide
gtggtggtcactgccgacagcg

SEQ ID NO: 17 - DNA, 25 bp, Artificial Sequence
  Synthetic oligonucleotide
cgcggcgtgctgaaaccaggcgttg

SEQ ID NO: 18 - DNA, 22 bp, Artificial Sequence
  Synthetic oligonucleotide
gcggttacgggtattgccgagc

SEQ ID NO: 19 - DNA, 50 bp, Artificial Sequence
  Synthetic oligonucleotide
acagttacccgtaacatttttaattcttgtattgtgggggcaccactttg

SEQ ID NO: 20 - DNA, 50 bp, Artificial Sequence
  Syhtnetic oligonucleotide
gttaaaacaagcgttaatgtagtcactacttatttgccttttttaataac

SEQ ID NO: 21 - DNA, 25 bp, Artificial Sequence
  Synthetic oligonucleotide
caaagtggtgcccccacaatacaag

SEQ ID NO: 22 - DNA, 26 bp, Artificial Sequence
  Artificial oligonucleotide
cgccaccagccgggcgattggcgtcc

SEQ ID NO: 23 - DNA, 28 bp, Artificial Sequence
  Synthetic oligonucleotide
ggcaaataagtagtgactacattaacgc

SEQ ID NO: 24 - DNA, 26 bp, Artificial Sequence
  Syhtnetic oligonucleotide
ccccgccggttgcagcgcgtgaccgc

SEQ ID NO: 25 - DNA, 23 bp, Artificial Sequence
  Synthetic oligonucleotide
ggcgctgaacgcctgctggcgcg

SEQ ID NO: 26 - DNA, 24 bp, Artificial Sequence
  Synthetic oligonucleotide
cccagggcgacaaccaccgccagg

SEQ ID NO: 27 - DNA, 27 bp, Artificial Sequence
  Synthetic oligonucleotide
cgctctacgcggcaagattgccgcggc

SEQ ID NO: 28 - DNA, 27 bp, Artificial Sequence
  Synthetic oligonucleotide
cgcttcgcccagtgccggacctacgcc

SEQ ID NO: 29 - DNA, 50 bp, Artificial Sequence
  Synthetic oligonucleotide
cgccaggcgtagtatcgcagcaggtaagatgattcaggagattttaaatg

SEQ ID NO: 30 - DNA, 50 bp, Artificial Sequence
  Synthetic oligonucleotide
gcgtggtgttgaaagccgatttttgaaaatcattcgcagcgctgatctga

SEQ ID NO: 31 - DNA, 25 bp, Artificial Sequence
  Synthetic oligonucleotide
ctcctgaatcatcttacctgctgcg

SEQ ID NO: 32 - DNA, 26 bp, Artificial Sequence
  Synthetic oligonucleotide
gcacatccgcccccatctctttgccc

SEQ ID NO: 33 - DNA, 25 bp, Artificial Sequence
  Synthetic oligonucleotide
caaaaatcggctttcaacaccacgc

SEQ ID NO: 34 - DNA, 27 bp, Artificial Sequence
  Synthetic oligonucleotide
ggccacaagatgttgaagtgcaggtgg

SEQ ID NO: 35 - DNA, 26 bp, Artificial Sequence
  Synthetic oligonucleotide
cctcactggtcaggtagacacctcgg

SEQ ID NO: 36 - DNA, 27 bp, Artificial Sequence
  Synthetic oligonucleotide
ccggcacgccttatgagctgcgtaagc

SEQ ID NO: 37 - DNA, 27 bp, Artificial Sequence
  Synthetic oligonucleotide
gccatcgcgcctcccatacctacctgc

SEQ ID NO: 38 - DNA, 25 bp, Artificial Sequence
  Synthetic oligonucleotide
ccaggcaccccgccctgccatgctc

SEQ ID NO: 39 - DNA, 50 bp, Artificial Sequence
  Synthetic oligonucleotide
tacagtgtgagaaagaattattttgactttagcggagcagttgaagaatg

SEQ ID NO: 40 - DNA, 50 bp, Artificial Sequence
  Synthetic oligonucleotide
gagttacctcccgatataaaaggaaggatttacagaatgtgacctaaggt

SEQ ID NO: 41 - DNA, 26 bp, Artificial Sequence
  Synthetic oligonucleotide
cattcttcaactgctccgctaaagtc

SEQ ID NO: 42 - DNA, 23 bp, Artificial Sequence
  Synthetic oligonucleotide
cgcgtgcaaccgtggtaagcacc

SEQ ID NO: 43 - DNA, 32 bp, Artificial Sequence
  Synthetic oligonucleotide
ccttccttttatatcgggaggtaactctcccg

SEQ ID NO: 44 - DNA, 23 bp, Artificial Sequence
  Synthetic oligonucleotide
cagcagttgcgtcggctgggtag

SEQ ID NO: 45 - DNA, 22 bp, Artificial Sequence
  Synthetic oligonucleotide
ggttctggctcgcggatggagc

SEQ ID NO: 46 - DNA, 25 bp, Artificial Sequence
  Synthetic oligonucleotide
ggcccatcgggcaggcacataaggc

SEQ ID NO: 47 - DNA, 24 bp, Artificial Sequence
  Synthetic oligonucleotide
gccaccatctgctgcatctcatcc

SEQ ID NO: 48 - DNA, 23 bp, Artificial Sequence
  Synthetic oligonucleotide
ggtggacggcaccggcgaagagg

SEQ ID NO: 49 - DNA, 50 bp, Artificial Sequence
  Synthetic oligonucleotide
ccacagcgcaaactaacgctaattttttacagatcaggttcacgactatg

SEQ ID NO: 50 - DNA, 50 bp, Artificial Sequence
  Synthetic oligonucleotide
ctttatccagccttttgcatatgctgcgtttacgctggcatcgcctcacg

SEQ ID NO: 51 - DNA, 50 bp, Artificial Sequence
  Synthetic oligonucleotide
catagtcgtgaacctgatctgtaaaaaattagcgttagtttgcgctgtgg

SEQ ID NO: 52 - DNA, 25 bp, Artificial Sequence
  Synthetic oligonucleotide
gccttgtgcggcgaatgcgggcgag

SEQ ID NO: 53 - DNA, 44 bp, Artificial Sequence
  Synthetic oligonucleotide
cgtgaggcgatgccagcgtaaacgcagcatatgcaaaaggctgg

SEQ ID NO: 54 - DNA, 26 bp, Artificial Sequence
  Synthetic oligonucleotide
caccccggatgcaatgacgctactgg

SEQ ID NO: 55 - DNA, 27 bp, Artificial Sequence
  Synthetic oligonucleotide
cctcactccacaacggaatctttcagg

SEQ ID NO: 56 - DNA, 28 bp, Artificial Sequence
  Synthetic oligonucleotide
gcgaccacctctgtcgccacggatgacg

SEQ ID NO: 57 - DNA, 24 bp, Artificial Sequence
  Synthetic oligonucleotide
gcgccagaaccgccgtattcagcg

SEQ ID NO: 58 - DNA, 24 bp, Artificial Sequence
  Synthetic oligonucleotide
ggtcacgatcctgccgatccgctg

SEQ ID NO: 59 - DNA, 25 bp, Artificial Sequence
  Syhtnetic oligonucleotide
atgagttctcaatttacgacgcctg

SEQ ID NO: 60 - DNA, 25 bp, Artificial Sequence
  Synthetic oligonucleotide
ttaacgcaccatgcacgggcgcttg

SEQ ID NO: 61 - DNA, 37 bp, Artificial Sequence
  Synthetic oligonucleotide
gaggaataaaccatgagttctcaatttacgacgcctg

SEQ ID NO: 62 - DNA, 39 bp, Artificial Sequence
  Synthetic oligonucleotide
ttgttctagaaagcttaacgcaccatgcacgggcgcttg

SEQ ID NO: 63 - DNA, 26 bp, Artificial Sequence
  Synthetic oligonucleotide
gtgtgggcactcgaccggaattatcg

SEQ ID NO: 64 - DNA, 23 bp, Artificial Sequence
  Synthetic oligonucleotide
ggtcgacggcgctattcagatcc

SEQ ID NO: 65 - DNA, 22 bp, Artificial Sequence
  Synthetic oligonucleotide
atggcgacacaatccagtcctg

SEQ ID NO: 66 - DNA, 31 bp, Artificial Sequence
  Synthetic oligonucleotide
ttaatgacggccgaaaacgggacgtttacgg

SEQ ID NO: 67 - DNA, 34 bp, Artificial Sequence
  Synthetic oligonucleotide
gaggaataaaccatggcgacacaatccagtcctg

SEQ ID NO: 68 - DNA, 45 bp, Artificial Sequence
  Synthetic oligonucleotide
ttgttctagaaagcttaatgacggccgaaaacgggacgtttacgg

SEQ ID NO: 69 - DNA, 21 bp, Artificial Sequence
  Synthetic oligonucleotide
atggaacagacctggcgctgg

SEQ ID NO: 70 - DNA, 25 bp, Artificial Sequence
  Synthetic oligonucleotide
ttaacggctaaagaaagcgcgctgg

SEQ ID NO: 71 - DNA, 33 bp, Artificial Sequence
  Synthetic oligonucleotide
gaggaataaaccatggaacagacctggcgctgg

SEQ ID NO: 72 - DNA, 39 bp, Artificial Sequence
  Synthetic oligonucleotide
ttgttctagaaagcttaacggctaaagaaagcgcgctgg

SEQ ID NO: 73 - DNA, 24 bp, Artificial Sequence
  Synthetic oligonucleotide
atgaagatcgtaaaggctgaagtt

SEQ ID NO: 74 - DNA, 28 bp, Artificial Sequence
  Synthetic oligonucleotide
ttaccagttccacagcgtgccatcttcc

SEQ ID NO: 75 - DNA, 36 bp, Artificial Sequence
  Synthetic oligonucleotide
gaggaataaaccatgaagatcgtaaaggctgaagtt

SEQ ID NO: 76 - DNA, 42 bp, Artificial Sequence
  Synthetic oligonucleotide
ttgttctagaaagcttaccagttccacagcgtgccatcttcc

SEQ ID NO: 77 - DNA, 38 bp, Artificial Sequence
  Synthetic oligonucleotide
gaggaataaaccatgtcgcaaactcctcgcaaactgcg

SEQ ID NO: 78 - DNA, 41 bp, Artificial Sequence
  Synthetic oligonucleotide
ttgttctagaaagcttagtgagagtgacgcggatcaccacg

SEQ ID NO: 79 - DNA, 3340 bp, Artificial Sequence
  Synthetic operon containing crtE, crtI, crtB genes from Erwinia uredorvora
atgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaataagctaaggaaaaacaaaatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgcgatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctaataagctt

SEQ ID NO: 80 - DNA, 43 bp, Artificial Sequence
  synthetic oligonucleotide
gaggaattaaccatgacggtctgcgcaaaaaaacacgttcatc

SEQ ID NO: 81 - DNA, 44 bp, Artificial Sequence
  synthetic oligonucleotide
gttcgggcccaagcttattagagcgggcgctgccagagatgcgc